(19) United States
(12) Patent Application Publication
(10) Pub. No.: US 2024/0342286 A1
(43) Pub. Date: Oct. 17, 2024

(54) NEAR-INFRARED (NIR) ABSORBING PHOTOSENSITIZERS

(71) Applicants: ORTA DOGU TEKNIK UNIVERSITESI, Ankara (TR); KOC UNIVERSITESI, Istanbul (TR)

(72) Inventors: Gorkem GUNBAS, Ankara (TR); Safacan KOLEMEN, Istanbul (TR); Osman KARAMAN, Ankara (TR); Gizem ATAKAN, Ankara (TR); Toghrul ALMAMMADOV, Istanbul (TR)

(73) Assignees: ORTA DOGU TEKNIK UNIVERSITESI, Ankara (TR); KOC UNIVERSITESI, Istanbul (TR)

(21) Appl. No.: 18/669,670

(22) Filed: May 21, 2024

Related U.S. Application Data
(62) Division of application No. 16/973,435, filed on Dec. 9, 2020.

(51) Int. Cl. A61K 41/00; C07F 5/02; C07F 7/08

(52) U.S. Cl. CPC A61K 41/0057; C07F 5/022; C07F 7/0814

(57) ABSTRACT

Near-infrared (NIR) absorbing photosensitizers for imaging and treatment of cancer in a photodynamic therapy (PDT) are provided. Specifically, the NIR photosensitizers are mitochondria targeted and water-soluble, and used as a cytotoxic drug in a photodynamic therapy of the cancer. The NIR photosensitizers are activated with wavelengths of light, wherein the light penetrates through a body not just a skin. Hence the NIR photosensitizers aim to transform PDT from being a specialized treatment to a generally applicable one. PDT is almost completely non-invasive compared to current treatment methods.

### CROSS REFERENCE TO THE RELATED APPLICATIONS

This application is a divisional of U.S. application Ser. No. 16/973,435 filed Dec. 9, 2020, which is the national stage entry of International Application No. PCT/TR2018/050322, filed on Jun. 25, 2018, the entire contents of which are incorporated herein by reference.

### TECHNICAL FIELD

The present invention relates to photosensitizers to be used for imaging and treatment of cancer by using photodynamic therapy (PDT).

### BACKGROUND

Photodynamic therapy (PDT) is a treatment that uses special drugs, called photosensitizing agents, along with light to kill cancer cells. The drugs only work after they have been activated or turned on by certain kinds of light.

In PDT, therapeutic action is satisfied by the generation of singlet oxygen (1O2) upon irradiation of well-designed PDT agents (photosensitizers or drugs) by light. Singlet oxygen is the excited form of molecular oxygen (3O2) and it is cytotoxic due to its high reactivity towards vital biomolecules. In addition to this oxidative damage triggered direct killing mode (apoptosis or necrosis) arises from 1O2 reactivity, PDT also restricts the flow of nutrients and oxygen to cancer cells by disturbing the vasculatures around tumor regions and at the same time activates the immune system against cancer cells. The advantages and importance of PDT are more pronounced when it is compared with widely used conventional chemotherapy and radiotherapy. Although these therapies are the primary choices in clinic practices with a huge market in pharmaceutical industry, they have numerous side effects and give serious damage to the immune system which make them patient unfriendly.

When considered from this aspect; PDT is highly promising, however its broader applicability in clinical treatments is restricted due to some problems. The most important one is the limited penetration of the irradiation light through tissues, which leaves the deeper tumors out from the scope of PDT. Studies have shown that red or near-IR (NIR) absorbing PDT agents should be employed to get the best penetration depth and to avoid the absorption of incoming light by biomolecules. Although, a very wide range of well-known fluorescent probe cores have been converted to PDT agents, red and near-IR absorbing PDT agents are still rare.

The second major problem of the PDT is the oxygen deficiency (hypoxia) in cancer cells. During PDT; singlet oxygen generation is highly dependent on oxygen, which decreases the efficacy of the treatment on hypoxic cancer cells. In addition to this, further oxygen consumption of PDT agents aggravates tumor hypoxia. Accordingly, some of the signaling pathways (ex: HIF-1) are turned-on under severe hypoxia and cause angiogenesis, proliferation and metastasis of hypoxic cancer cells.

The third problem that PDT should overcome is the selective generation of singlet oxygen only in cancer cells without giving harm to healthy cells by employing water soluble and activatable drugs.

Most of the initial photosensitizers (first-generation) and FDA approved drugs are based on a porphyrin skeleton. Although they are really satisfying in terms of singlet oxygen generation and cytotoxicity, almost all of them have water solubility and photostability problems. Later phthalocyanine derivatives were introduced as promising photosensitizers for PDT applications, however synthetic problems and lack of control on photophysical properties restricted their use.

At this point, dipyrrometheneboron difluoride (BODIPY) core appeared to be good candidate due to its tunable photophysical properties, ease of synthesis and modification. Parent BODIPY core is highly hydrophobic and absorbs around 500 nm. Thus; for PDT applications, the core should be modified with water-soluble groups and p-conjugation should be extended in order to have a red-absorbing photosensitizer.

In literature, it is possible to find different version of BODIPYs serving as PDT drugs, however a water-soluble, red-absorbing and at the same time mitochondria targeted BODIPY based PDT agent has not been introduced before. Mitochondria is an important target in order to address hypoxia problem of PDT. It is known that oxygen mechanism is highly evaluated in mitochondria, and this high level of oxygen can be used by PDT agents to generate singlet oxygen efficiently even in aggressive solid tumors, in which severe hypoxia can be observed.

Also; resorufin cores have been extensively used as fluorescent agents for bio-imaging during the last decades, but have not been employed in PDT studies. Resorufin skeleton is quite suitable for PDT applications as it is intrinsically red-absorbing without any modification, photostable and water-soluble.

There is no doubt that revealing new PDT agent cores holding aforementioned properties is highly critical but the most promising and breakthrough outcomes will come from the studies aimed at gaining precise control over the location and rate of the cytotoxic singlet oxygen generation. To that end, most of the new generation PDT drugs are so called “activatable”. They remain in a passive state (no cytotoxicity) even under light irradiation and are activated at the site where therapy is desired by cancer cell associated stimuli. In literature, the most widely used approach is to take advantage of the acidic microenvironment of tumors by designing acidic pH activatable PDT drugs. Another common design principle is to employ bio-thiols that are elevated in cancer cells such as glutathione (GSH) as an activating biological input.

As studies on cancer biology and signalling pathways increase, role of many different enzymes in cancer have been uncovered during last decades. Consequently enzymes that are overexpressed in cancer cells appeared to be good candidates for activatable PDT drug designs. Accordingly, caspase-3, cathepsin B, nitroreductase and b-galactosidase activated PDT drugs were introduced with conventional PDT agent cores, however enzyme-activated agents are still rare and their potential can be further evaluated with the help of new generation PDT drug cores.

In this invention, enzyme activated resorufin based PDT agents and BODIPY based PDT agents are synthesized to use in treatment of cancer.

### SUMMARY

The invention is about new photosensitizers for wide variety of cancer cells treatment by using photodynamic therapy. These photosensitizers can be activated with wavelengths of light that can penetrate through the body not just skin. Hence the present invention aims to transform PDT from being a specialized treatment to a generally applicable one. PDT is almost completely non-invasive compared to current treatment methods. Additionally due to its non-invasive nature treatment, it can be performed with much shorter intervals compared to chemotherapy. From the perspective of financials, it is known that chemotherapy reagents are expensive. The photosensitizers developed in this invention designed in a way that target compounds can be attained with very small number of steps with cost effective reagents/reactants. Most of the known literature compounds require long synthetic approaches towards achieving some of properties (water solubility, targeting, Near-IR absorption, etc.). The synthetic approaches described in this invention require a small number of steps from commercially available starting materials. Core structures can be attained in 5-6 steps. This creates a significant opportunity for commercialization of these drugs as PDT agents. In addition to this, the BODIPY derivatives synthesized in this invention are addressing the critical light penetration depth and hypoxia problems of PDT and theses BODIPY based photosensitizers are water-soluble, red-absorbing and at the same time mitochondria targeted.

### BRIEF DESCRIPTION OF THE DRAWINGS

FIG. 1 Absorption of selenophene-substituted BODIPY derivative.

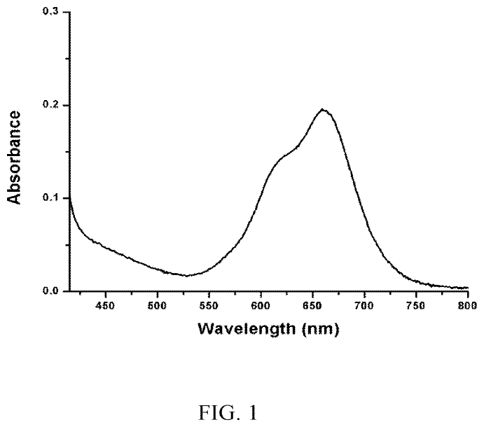

### DETAILED DESCRIPTION OF THE EMBODIMENTS

In the present invention, BODIPY based and resorufin based photosensitizers are synthesized for use in photodynamic therapy of cancer.

### BODIPY-Based Photosensitizers

These sensitizers are modified with selenophene (Se-BOD) or iodo-selenophene (SeI-BOD) units at 2 and 6 positions of the BODIPY core in order to facilitate intersystem crossing (ISC) upon external light irradiation as a result of heavy atom (selenium or selenium (Se) and iodine (I)) mediated spin-orbit coupling. These BODIPY derivatives absorb red-light around 650 nm and highly water soluble with the help of methyl pyridinium moeties such that only 0.1% DMSO as a co-solvent is needed to get clear solutions. Methyl pyridinium groups contribute to three important issues. First, these groups improve the water solubility of the PDT drug as mentioned. Second, they cause a red-shift in the absorption signal and finally they direct the drug to mitochondria (mitochondria targeting). It is known in the literature that most of the solid tumors are oxygen deficient (hypoxic). This hypoxic condition within the tumor cells is a real challenge for PDT drugs since the therapy itself consumes oxygen to generate cytotoxic singlet oxygen, which makes PDT a self-restricting method. One way to address this drawback is to direct PDT drugs to mitochondria, where the oxygen metabolism is high. Common strategy for mitochondria targeting is to use cationic species such as triphenylphosphine and methyl/hexyl pyridinium moieties. In this invention it is decided to use preferably methyl pyridinium, since it has several aforementioned advantages and it is easy to synthesize.

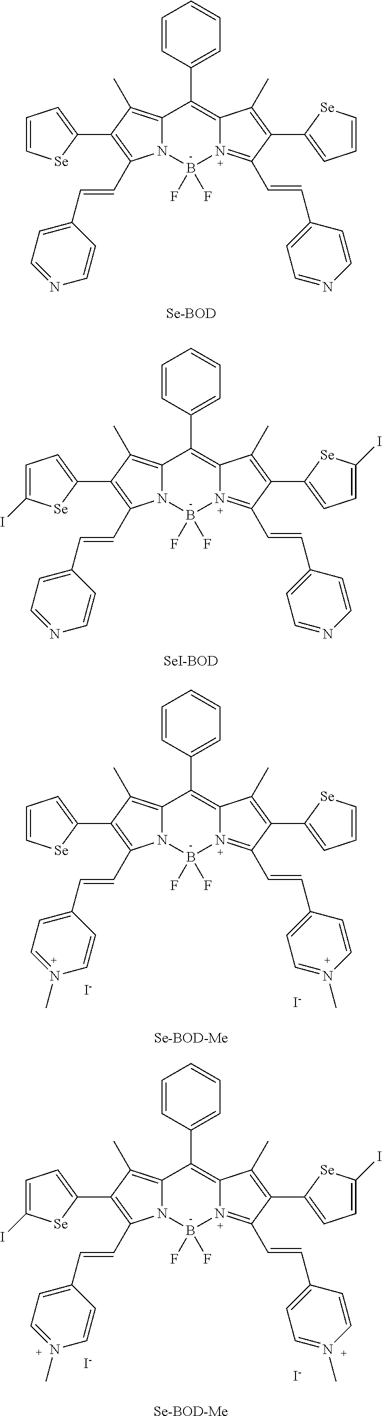

All these features mentioned above combined with selenophene incorporation into the BODIPY results in a PDT drug with high water solubility, absorption in the near IR region (>650 nm) increased singlet oxygen generation efficiency (compared to bromine substitution) and mitochondria targeting abilities. Hence, the system described here in this invention is a patient ready cancer treatment PDT drug. Fluorescence quantum yield of the sensitizers (8.5% for Se-BOD) are low as ISC pathway is active. Singlet oxygen quantum yield for Se-BOD was calculated as 18% in phosphate buffered saline (PBS) (pH 7.4, 10 mM) with 0.1% dimethyl sulfoxide (DMSO).

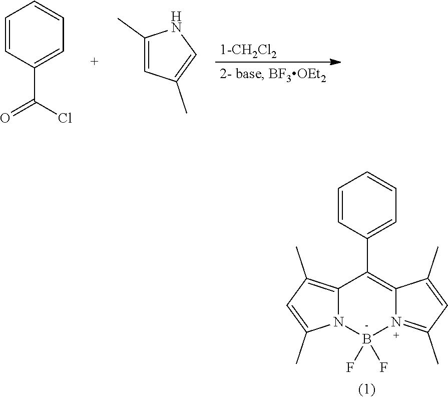

Commercially available benzoyl chloride (C₆H₅COCl) and 2,4-dimethylpyrrole are dissolved in methylene chloride (CH₂Cl₂) and stirred at 0° C. to 40° C. until starting material finished. Then proper amine base and boron trifluoride diethyl etherate (BF₃·OEt₂) is added and stirred 6-12 h. Then washed with water and purified by column chromatography and Compound 1 is synthesized successfully.

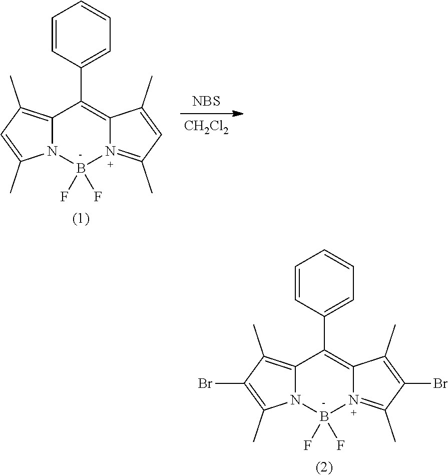

To a solution of Compound 1 in CH₂Cl₂, a solution of N-bromosuccinimide (NBS) in CH₂Cl₂ is added and stirred 2-4 h at 0-40° C. then washed with water and brine and purified by column chromatography and Compound 2 is synthesized successfully.

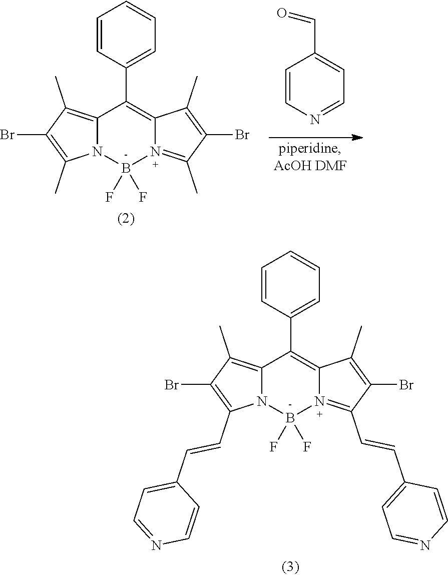

Compound 2 dissolved in dry N,N-dimethylformamide (DMF) and 4-pyridinecarboxyaldehyde, piperidine and acetic acid (AcOH) are added. Reaction mixture is stirred for 1-2 h at 0°−40° C., then solvent is evaporated and purified by column chromatography to yield Compound 3 successfully.

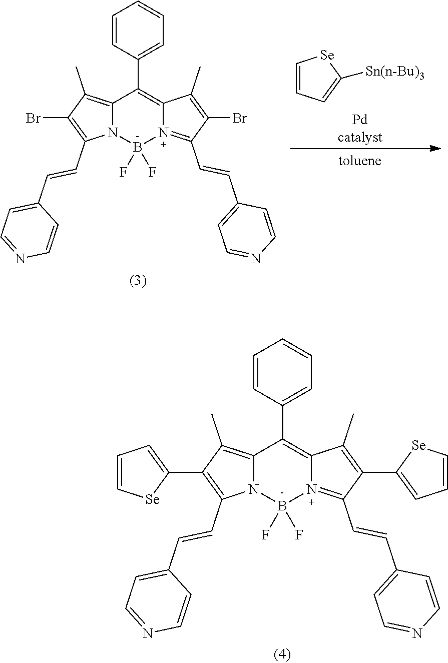

Compound 3 is dissolved in toluene, then stannylated selenophene and palladium (Pd) catalyst (Pd(PPh₃)Cl2, Pd(OAc)2 etc.) are added and stirred for 4-6 days at 100-140° C. Then it is washed with potassium fluoride (KF) solution and purified by column chromatography to yield Compound 4.

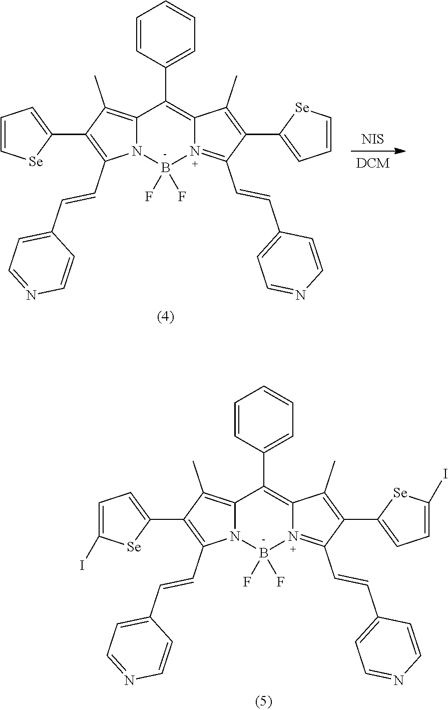

To a solution of Compound 4 in CH₂Cl₂, a solution of N-iodosuccinimide (NIS) in CH₂Cl₂ is added. Reaction mixture is stirred for 12-24 h at 0-40° C. and washed with water and sodium bicarbonate (NaHCO₃). Purification is performed by column chromatography to yield Compound 5.

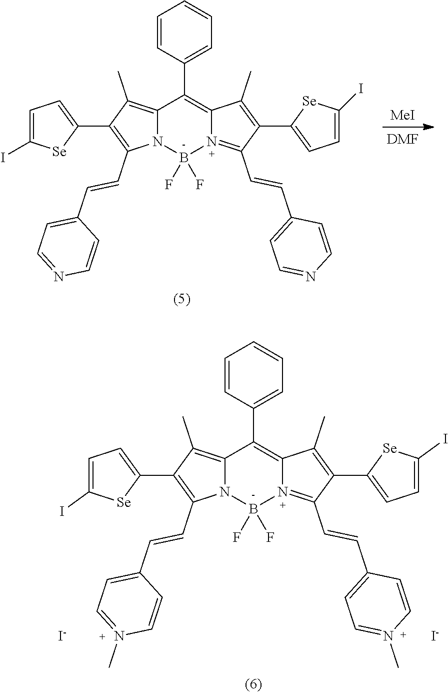

To a solution of Compound 5 in dry DMF, methyliodide is added and stirred 1-3 days at 0-40° C. The reaction mixture is precipitated with diethylether with the presence of DMF and Compound 6 is synthesized successfully.

In this invention heavy atoms are used in the synthesis of photosensitizer derivatives. Presence of heavy atoms is a requirement for a PDT drug. Once the sensitizers are excited with light to a singlet excited state presence of heavy atoms results in inter system crossing and a triplet excited state is generated. This triplet excited states is responsible for the generation of triplet oxygen from the oxygen present in the cells. Iodine as a heavy atom favors inter-system crossing by itself. In this invention, the aim is to increase the number of heavy atoms on the BODIPY core in order to increase the singlet oxygen generation yield of the drug synthesized in this invention through synergistic effect of selenium and iodine. Upon generation of singlet oxygen, it reacts with vital bio-molecules causing oxidative damage, which results in cell death. In addition to direct killing of cancer cells, PDT also restricts the follow of nutrients and oxygen to cancer cells by damaging the vasculatures around tumor regions and at the same time activates immune system against cancer cells.

### General Structure of Synthesized BODIPY-Based Photosensitizers

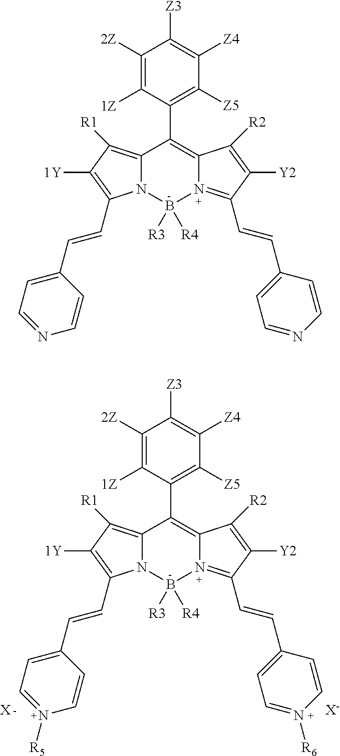

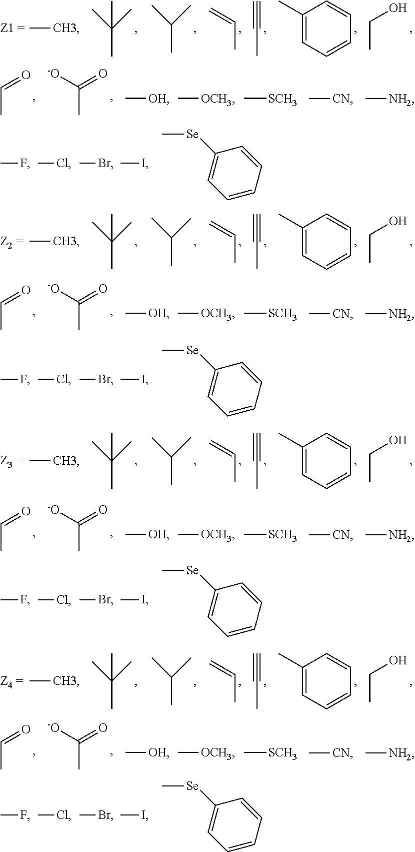

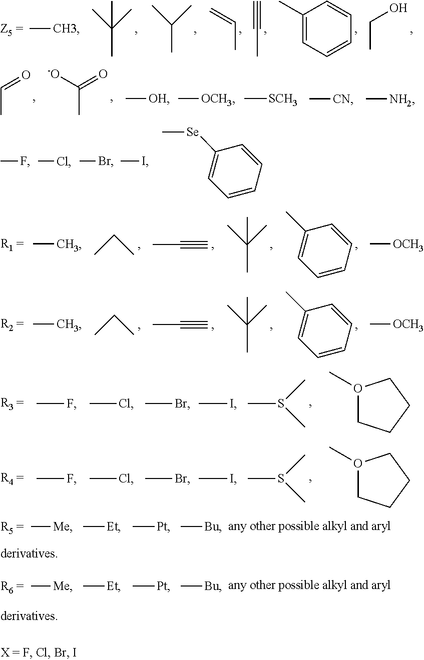

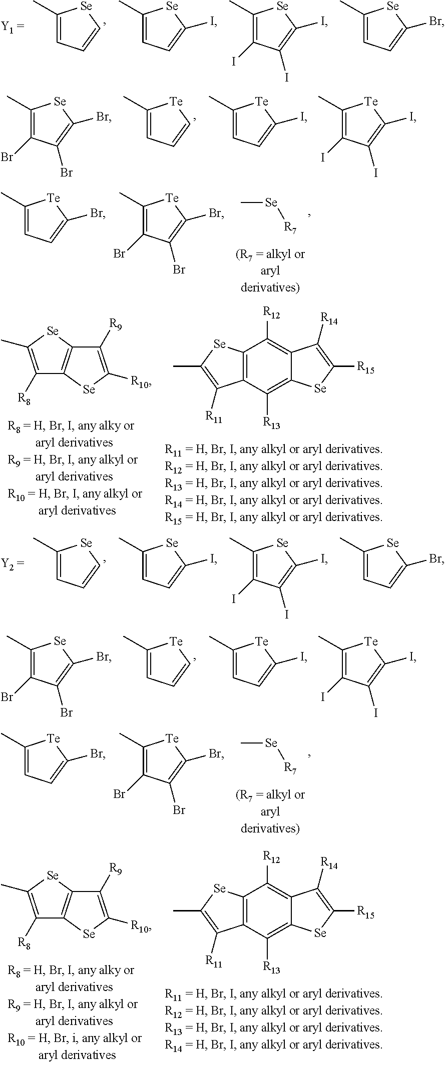

### Resorufin-Based Photosensitizers

Resorufin derivatives are introduced as photosensitizers for photodynamic therapy of cancer for the first time in this invention. Iodine is incorporated to the core structure as a heavy atom in order to favor ISC. These resorufin-based sensitizers are enzyme activated in such a way that cytotoxicity can only be observed in cancer cells. In the case of PDT agent, a sugar cage group-D-galactopyranoside is attached to the iodinated-resorufin core and this modification shifted the absorption maximum of the drug to 500 nm. In literature it is known that 8-D-galactopyranoside sugar cage can be removed selectively with b-galactosidase enzyme, which is overexpressed in several cancer types including ovarian and liver cancers. As a result of reaction between b-galactosidase and the PDT agent sugar cage removed and the absorption maximum of the resorufin core is red-shifted to 600 nm. Consequently, by adjusting the excitation wavelength during the PDT action to 600 nm, the PDT agent becomes cytotoxic only in cancer cells in which the b-galactosidase concentration is high, and on the contrary no cytotoxicity is observed in healthy cells as the PDT agent does not absorb the irradiation light at 600 nm. PDT drug functions are similar, but the cage group is replaced by a propanamine, which can be selectively cleaved by monoamine oxidase (MAO) enzyme. The PDT drug does not show any cytotoxicity in health cells, but it is highly cytotoxic in cancer cells upon 600 nm irradiation.

A new fluorophore core, silicon-substituted resorufin (Turkey Red) is designed and synthesized in this invention. It has red shifted absorption and emission signals compared to conventional resorufin (oxygen-substituted resorufin). This new core is also converted to a cytotoxic photosensitizer for PDT applications simply by decorating the core structure with heavy atoms such as bromine or iodine.

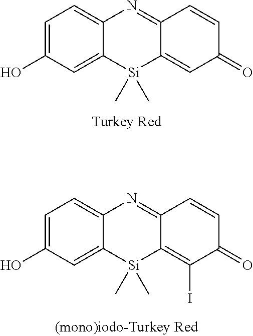 [SMILES: C[Si]1(C)C2=CC(=O)C=CC2=Nc2ccc(O)cc21 ; C[Si]1(C)C2=C(I)C(=O)C=CC2=Nc2ccc(O)cc21]

Replacing oxygen with silicon and phosphine oxide is causing approximately 100 nm red shift in the absorption spectra of the cores which is quite unusual. Just replacing one atom of the core results in a very strong shift which in turn makes these cores quite valuable for PDT applications. The change of sp² carbon atom with nitrogen in the fluorescein core results in resorufin. Here almost a 100 nm shift is also observed. One of the main ideas in this invention is combining both sp2 carbon to nitrogen change and oxygen to silicon/phosphine oxide change in the same core and has a combined effect of almost 200 nm red shift. This results in a record red shift with simply modifying the core structures which is very valuable since other approaches to attain NIR absorption requires many synthetic steps and transformations which make the drug candidates extremely expensive.

### The Synthesis of Resorufin-Based Photosensitizer of Formula Below:

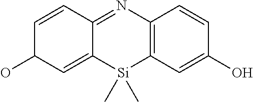 [SMILES: C[Si]1(C)C2=CC(O)C=CC2=Nc2ccc(O)cc21]

Commercially available bis(4-methoxyphenyl)amine is treated with % 30-50 hydrogen bromide (HBr) and Aliquat 336 at 70-120° C. for 12-48 hours. Mixture is diluted with water and extracted with ethyl acetate (EtOAc). The solvent is evaporated and residue is washed with hexane to get the demethylated product 4,4′-azanediyldiphenol.

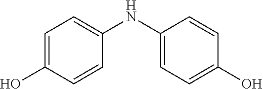 [SMILES: Oc1ccc(Nc2ccc(O)cc2)cc1]

### 4,4′-azanediyldiphenol

4,4′-azanediyldiphenol is brominated with NBS (2-4 equivalents) in acetonitrile at (−10)−25° C. The mixture is stirred for 2-8 hours. Solvent is removed and the residue is dissolved in THF. The mixture is filtered through Celite and purified by column chromatography to give the dibrominated product 4,4′-azanediylbis(3-bromophenol).

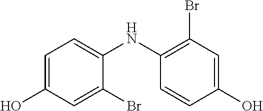 [SMILES: Oc1ccc(Nc2ccc(O)cc2Br)c(Br)c1]

### 4,4′-azanediylbis(3-bromophenol

4,4′-azanediylbis(3-bromophenol) is treated with sodium hydride (NaH, 4-6 equivalents) in DMF at (−10)-25° C. Mixture is stirred for 1-2 hours and then benzyl bromide (4-6 equivalents) is added and the mixture is heated to 70-100° C. for 4-18 hours. Water is added and the mixture was extracted with diethyl ether (Et₂O). The solvent is evaporated and the product shown in below is attained by column chromatography.

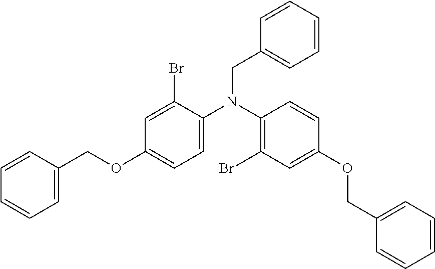 [SMILES: Brc1cc(OCc2ccccc2)ccc1N(Cc1ccccc1)c1ccc(OCc2ccccc2)cc1Br]

### N-benzyl-4-(benzyloxy)-N-(4-(benzyloxy)-2-bromophenyl)-2-bromoaniline

N-benzyl-4-(benzyloxy)-N-(4-(benzyloxy)-2-bromophenyl)-2-bromoaniline is dissolved in Et₂O and the mixture is cooled to −80° C. to −20° C. n-butyllithium (n-BuLi) is added and the mixture is stirred for 1 hour. Then dichloro(dimethyl)silane (SiMe₂Cl₂) is added and the mixture is gradually warmed to room temperature. Et₂O and water are added into the mixture and the organic phase is separated. Solvent is evaporated and the residue is purified by column chromatography to give 5-benzyl-2,8-bis(benzyloxy)-10,10-dimethyl-5,10-dihydrodibenzo[b,e][1,4]azasiline.

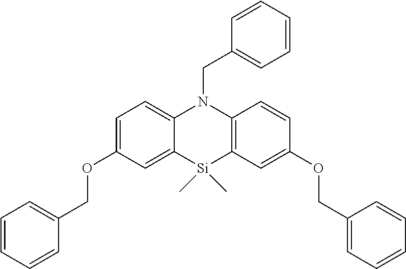 [SMILES: C[Si]1(C)c2cc(OCc3ccccc3)ccc2N(Cc2ccccc2)c2ccc(OCc3ccccc3)cc21]

### 5-benzyl-2,8-bis(benzyloxy)-10,10-dimethyl-5,10-dihydrodibenzo[b,e][1,4]azasiline

5-benzyl-2,8-bis(benzyloxy)-10,10-dimethyl-5,10-dihydrodibenzo[b,e][1,4]azasiline is dissolved in ethanol and palladium on carbon (Pd/C) is added at 0-30° C. The system is vacuumed and filled with hydrogen (H₂) gas. The mixture is stirred for 4-12 hours. The mixture is diluted with ethanol and filtered through celite. The solvent is evaporated and the mixture is purified by column chromatography.

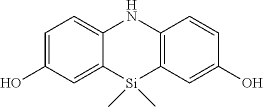 [SMILES: C[Si]1(C)c2cc(O)ccc2Nc2ccc(O)cc21]

### 10,10-dimethyl-5,10-dihydrodibenzo[b,e][1,4]azasiline-2,8-diol

10,10-dimethyl-5,10-dihydrodibenzo[b,e][1,4]azasiline-2,8-diol is dissolved in DCM and an appropriate oxidant (DDQ, Dess-Martin, etc) is added at (−10)-25° C. The mixture is stirred for 2-12 hours and the mixture is diluted with dichloromethane (DCM) and filtered through celite. The residue is washed with water and the organic solvent is evaporated. The residue was purified by column chromatography to get the title compound of formula below.

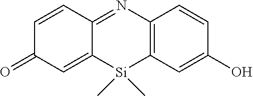 [SMILES: C[Si]1(C)C2=CC(=O)C=CC2=Nc2ccc(O)cc21]

### 8-hydroxy-10,10-dimethyldibenzo[b,e][1,4]azasilin-2 (10H)-one

### The Synthesis of Resorufin-Based Photosensitizer of Formula Below:

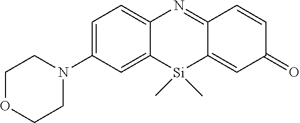 [SMILES: C[Si]1(C)C2=CC(=O)C=CC2=Nc2ccc(N3CCOCC3)cc21]

10,10-dimethyl-5,10-dihydrodibenzo[b,e][1,4]azasiline-2,8-diol is dissolved in DCM. Pyridine is added and the mixture is cooled to (−20)-10° C. Trifilic anhydride is then added and the mixture is stirred for 1-4 hours. The mixture is diluted with DCM and washed with water. The organic solvent is evaporated and the product shown in below is attained.

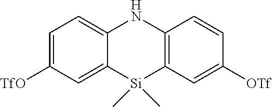 [SMILES: C[Si]1(C)c2cc(OS(=O)(=O)C(F)(F)F)ccc2Nc2ccc(OS(=O)(=O)C(F)(F)F)cc21]

### 10,10-dimethyl-5,10-dihydrodibenzo[b,e][1,4]azasiline-2,8-diyl bis(trifluoromethanesulfonate

10,10-dimethyl-5,10-dihydrodibenzo[b,e][1,4]azasiline-2,8-diyl bis(trifluoromethanesulfonate) is dissolved in toluene and Pd(OAc)2 and an appropriate ligand (Xantphos, DavePhos, XPhos, etc) is added. The system is purged with argon and the appropriate amine (in this example morpholine) is added. The mixture is stirred at 50-100° C. for 12-36 hours. The reaction was diluted with EtOAc and washed with water. The solvent was evaporated. The residue was treated with Et4NOH in MeOH the residue was purified by column chromatography.

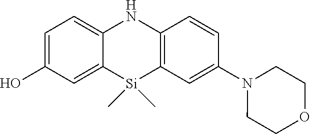 [SMILES: C[Si]1(C)c2cc(O)ccc2Nc2ccc(N3CCOCC3)cc21]

### 10,10-dimethyl-8-morpholino-5,10-dihydrodibenzo[b,e][1,4]azasilin-2-ol

10,10-dimethyl-8-morpholino-5,10-dihydrodibenzo[b,e][1,4]azasilin-2-ol was dissolved in DCM and an appropriate oxidant (DDQ, Dess-Martin, etc) was added at −10-25° C. The mixture was stirred for 2-12 hours and the mixture was diluted with DCM and filtered through Celite. The residue was washed with water and the organic solvent was evaporated. The residue was purified by column chromatography to get the title compound.

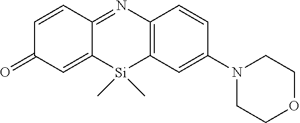 [SMILES: C[Si]1(C)C2=CC(=O)C=CC2=Nc2ccc(N3CCOCC3)cc21]

### 10,10-dimethyl-8-morpholinodibenzo[b,e][1,4]azasilin-2 (10H)-one

### The Synthesis of Resorufin-Based Photosensitizer of Formula Below:

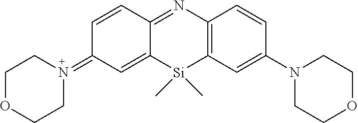

10,10-dimethyl-5,10-dihydrodibenzo[b,e][1,4]azasiline-2,8-diyl bis(trifluoromethanesulfonate) was dissolved in toluene and Pd(OAc)2 and an appropriate ligand (Xantphos, DavePhos, XPhos, etc) was added. The system was purged with argon and the appropriate amine (in this example dime was added. The mixture was stirred at 50-100° C. for 12-36 hours. The reaction was diluted with EtOAc and washed with water. The solvent was evaporated and the residue was purified by column chromatography.

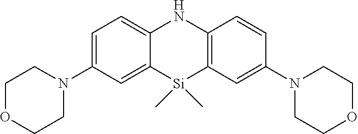 [SMILES: C[Si]1(C)c2cc(N3CCOCC3)ccc2Nc2ccc(N3CCOCC3)cc21]

### 10,10-dimethyl-8-morpholino-5,10-dihydrodibenzo[b,e][1,4]azasilin-2-ol

10,10-dimethyl-8-morpholino-5,10-dihydrodibenzo[b,e][1,4]azasilin-2-ol was dissolved in DCM and an appropriate oxidant (DDQ, Dess-Martin, etc) was added at −10-25° C. The mixture was stirred for 2-12 hours and the mixture was diluted with DCM and filtered through Celite. The residue was washed with water and the organic solvent was evaporated. The residue was purified by column chromatography to get the title compound.

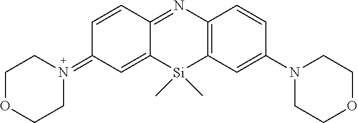

### 10,10-dimethyl-8-morpholinodibenzo[b,e][1,4]azasilin-2 (10H)-one

### The Synthesis of Resorufin-Based Photosensitizer of Formula Below:

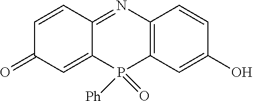 [SMILES: O=C1C=CC2=Nc3ccc(O)cc3P(=O)(c3ccccc3)C2=C1]

N-benzyl-4-(benzyloxy)-N-(4-(benzyloxy)-2-bromophenyl)-2-bromoaniline was dissolved in Et2O and the mixture was cooled to −80 to −20° C. n-BuLi was added and the mixture was stirred for 1 hour. Then PPhCl2 was added and the mixture was gradually warmed to room temperature. H₂O₂ was added and the mixture was stirred for 1-3 hours. EtOAc was added and water was added the organic phase was separated. Solvent was evaporated and the residue purified by column chromatography to give the title compound.

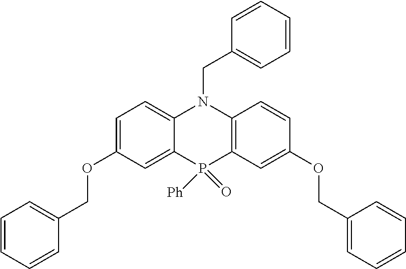 [SMILES: O=P1(c2ccccc2)c2cc(OCc3ccccc3)ccc2N(Cc2ccccc2)c2ccc(OCc3ccccc3)cc21]

### 5-benzyl-2,8-bis(benzyloxy)-10-phenyl-5H-phenophosphazinine 10-oxide

5-benzyl-2,8-bis(benzyloxy)-10-phenyl-5H-phenophosphazinine 10-oxide was dissolved in Ethanol and Pd/C was added at 0-30° C. The system was vacuumed and filled with H₂ gas. The mixture was stirred for 4-12 hours. The mixture was diluted with Ethanol and filtered through Celite. The solvent was evaporated and the mixture was purified by column chromatography.

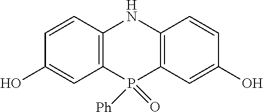 [SMILES: O=P1(c2ccccc2)c2cc(O)ccc2Nc2ccc(O)cc21]

### 2,8-dihydroxy-10-phenyl-5H-phenophosphazinine 10-oxide

2,8-dihydroxy-10-phenyl-5H-phenophosphazinine 10-oxide was dissolved in DCM and an appropriate oxidant (DDQ, Dess-Martin, etc) was added at −10-25° C. The mixture was stirred for 2-12 hours and the mixture was diluted with DCM and filtered through Celite. The residue was washed with water and the organic solvent was evaporated. The residue was purified by column chromatography to get the title compound.

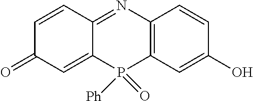 [SMILES: O=C1C=CC2=Nc3ccc(O)cc3P(=O)(c3ccccc3)C2=C1]

### 8-hydroxy-10-phenyl-2H-phenophosphazinin-2-one 10-oxide

### The Synthesis of Resorufin-Based Photosensitizer of Formula Below:

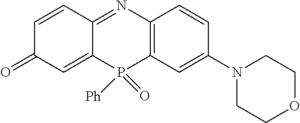 [SMILES: O=C1C=CC2=Nc3ccc(N4CCOCC4)cc3P(=O)(c3ccccc3)C2=C1]

2,8-dihydroxy-10-phenyl-5H-phenophosphazinine 10-oxide was dissolved in DCM. Pyridine was added and the mixture was cooled to −20-10° C. Trifilic anhydride was then added and the mixture was stirred for 1-4 hours. The mixture was diluted with DCM and washed with water. The organic solvent was evaporated and the title product was attained.

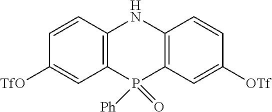 [SMILES: O=P1(c2ccccc2)c2cc(OS(=O)(=O)C(F)(F)F)ccc2Nc2ccc(OS(=O)(=O)C(F)(F)F)cc21]

### 10-oxido-10-phenyl-5H-phenophosphazinine-2,8-diyl bis(trifluoromethanesulfonate

10-oxido-10-phenyl-5H-phenophosphazinine-2,8-diyl bis(trifluoromethanesulfonate) was dissolved in toluene and Pd(OAc)2 and an appropriate ligand (Xantphos, DavePhos, XPhos, etc) was added. The system was purged with argon and the appropriate amine (in this example morpholine) was added. The mixture was stirred at 50-100° C. for 12-36 hours. The reaction was diluted with EtOAc and washed with water. The solvent was evaporated. The residue was treated with Et4NOH in MeOH the residue was purified by column chromatography.

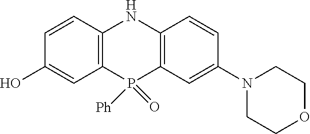 [SMILES: O=P1(c2ccccc2)c2cc(O)ccc2Nc2ccc(N3CCOCC3)cc21]

### 2-hydroxy-8-morpholino-10-phenyl-5H-phenophosphazinine 10-oxide

2-hydroxy-8-morpholino-10-phenyl-5H-phenophosphazinine 10-oxide was dissolved in DCM and an appropriate oxidant (DDQ, Dess-Martin, etc) was added at −10-25° C. The mixture was stirred for 2-12 hours and the mixture was diluted with DCM and filtered through Celite. The residue was washed with water and the organic solvent was evaporated. The residue was purified by column chromatography to get the title compound.

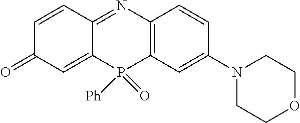 [SMILES: O=C1C=CC2=Nc3ccc(N4CCOCC4)cc3P(=O)(c3ccccc3)C2=C1]

### 8-morpholino-10-phenyl-2H-phenophosphazinin-2-one 10-oxide

### The Synthesis of Resorufin-Based Photosensitizer of Formula Below:

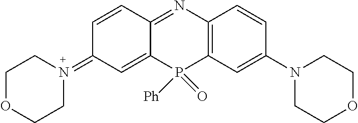

10-oxido-10-phenyl-5H-phenophosphazinine-2,8-diyl bis(trifluoromethanesulfonate) was dissolved in toluene and Pd(OAc)2 and an appropriate ligand (Xantphos, DavePhos, XPhos, etc) was added. The system was purged with argon and the appropriate amine (in this example morpholine) was added. The mixture was stirred at 50-100° C. for 12-36 hours. The reaction was diluted with EtOAc and washed with water. The solvent was evaporated. The residue was treated with Et4NOH in MeOH the residue was purified by column chromatography.

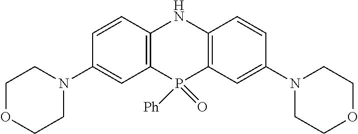 [SMILES: O=P1(c2ccccc2)c2cc(N3CCOCC3)ccc2Nc2ccc(N3CCOCC3)cc21]

### 2,8-dimorpholino-10-phenyl-5H-phenophosphazinine 10-oxide

2,8-dimorpholino-10-phenyl-5H-phenophosphazinine 10-oxide was dissolved in DCM and an appropriate oxidant (DDQ, Dess-Martin, etc) was added at −10-25° C. The mixture was stirred for 2-12 hours and the mixture was diluted with DCM and filtered through Celite. The residue was washed with water and the organic solvent was evaporated. The residue was purified by column chromatography to get the title compound.

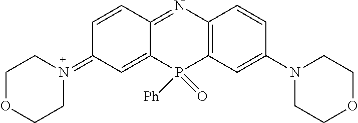

### 4-(8-morpholino-10-oxido-10-phenyl-2H-phenophosphazinin-2-ylidene) morpholin-4-ium

### General Structure of Synthesized Resorufin-Based Photosensitizers

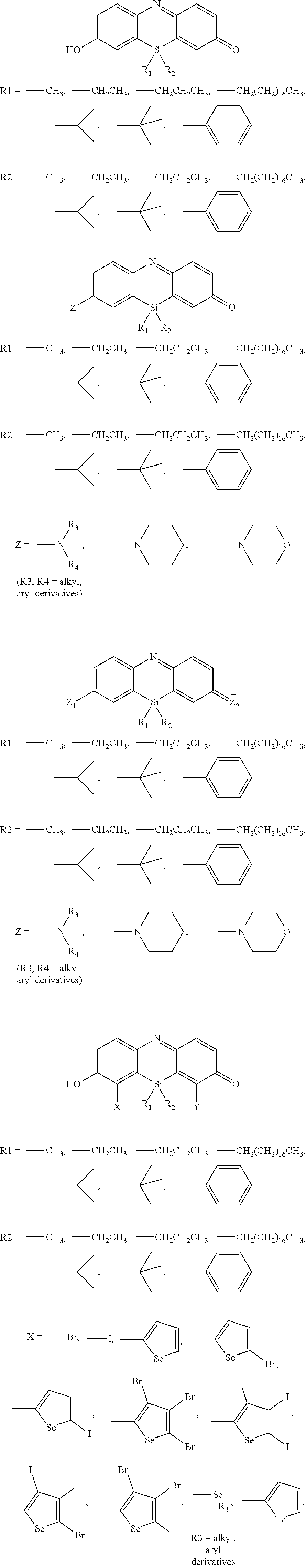

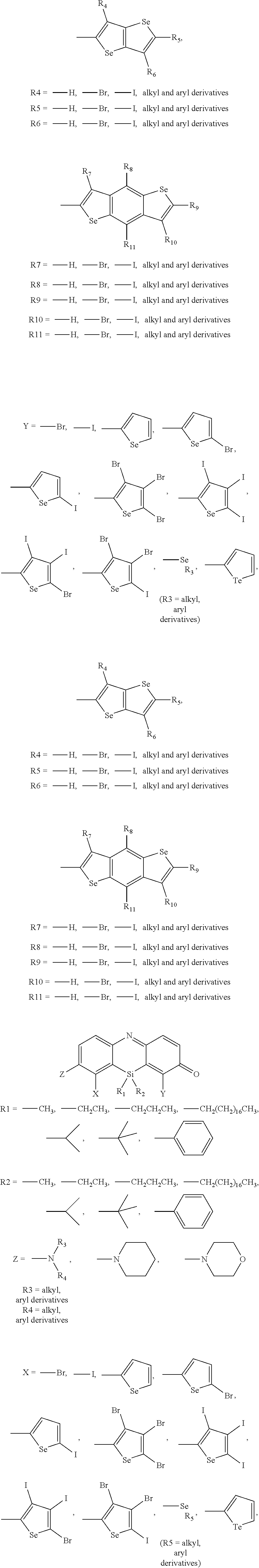

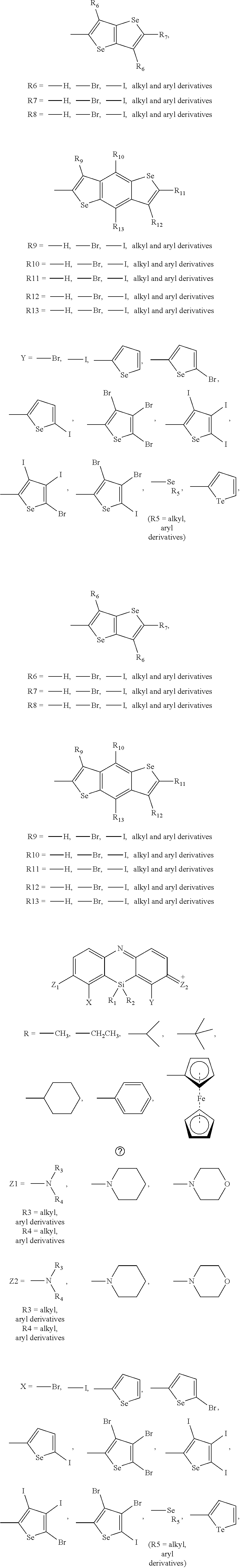

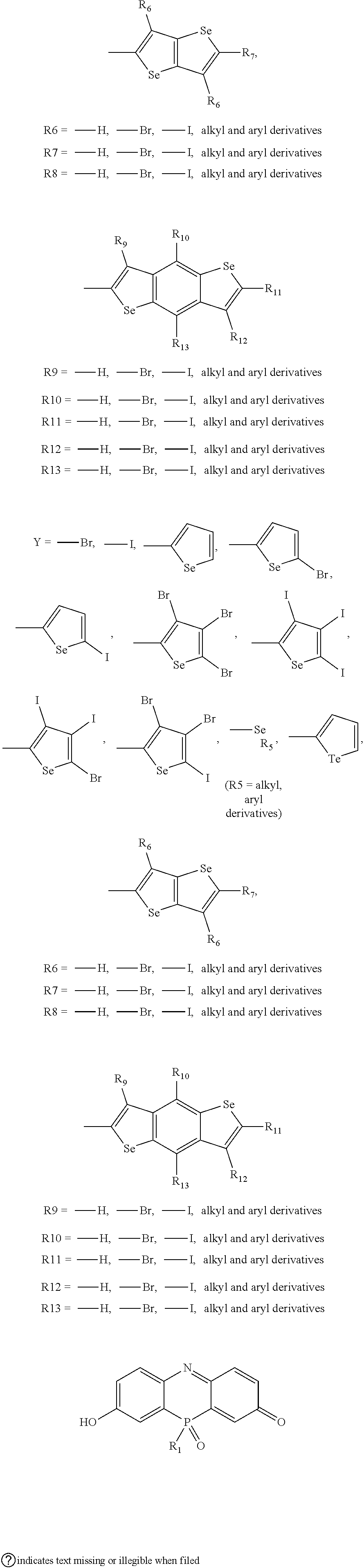

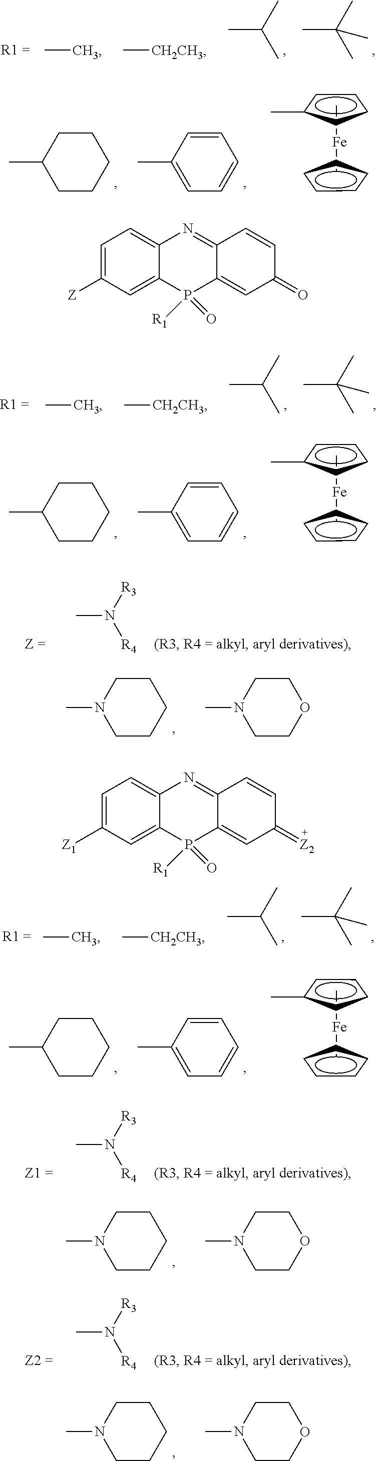

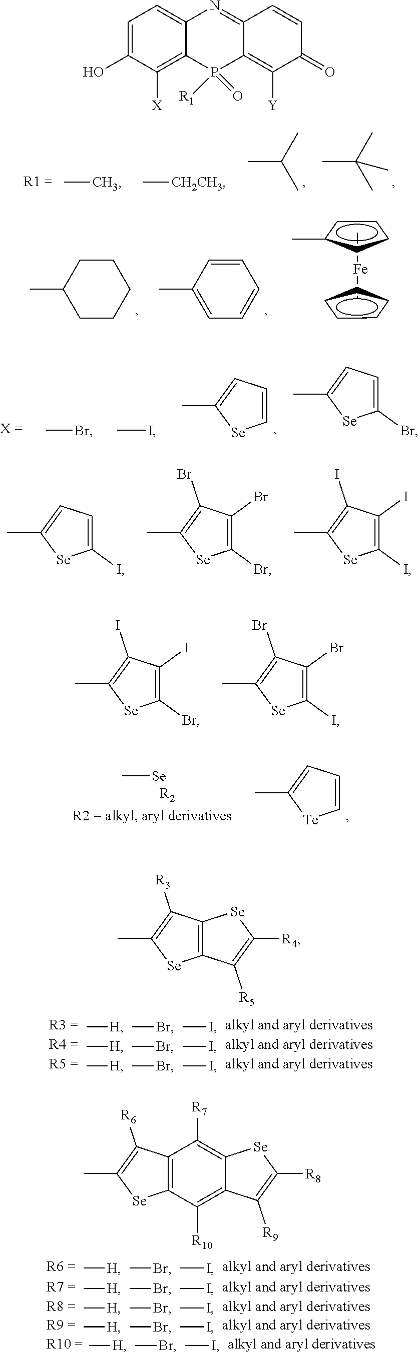

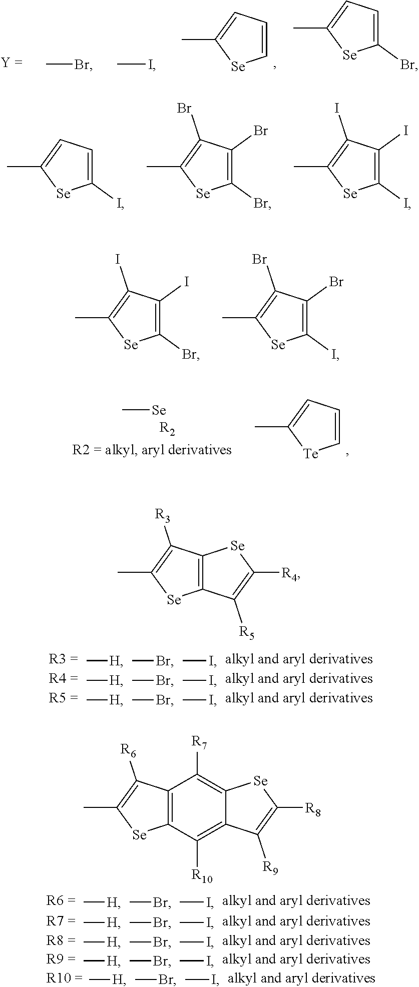

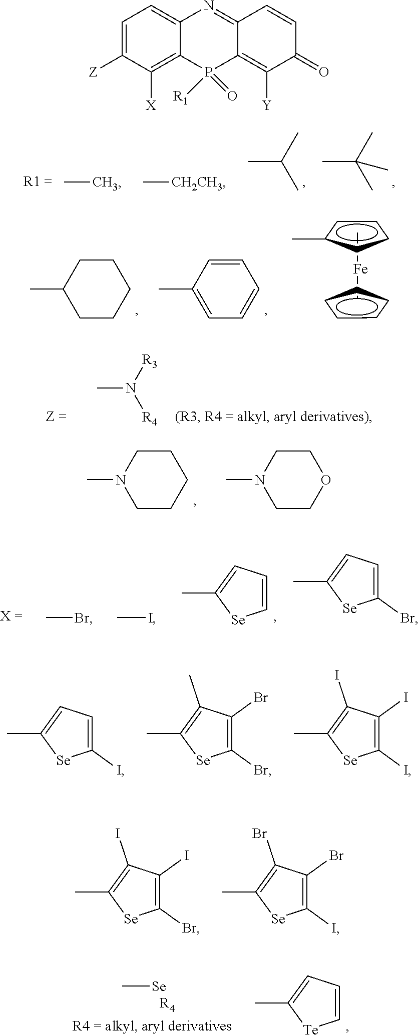

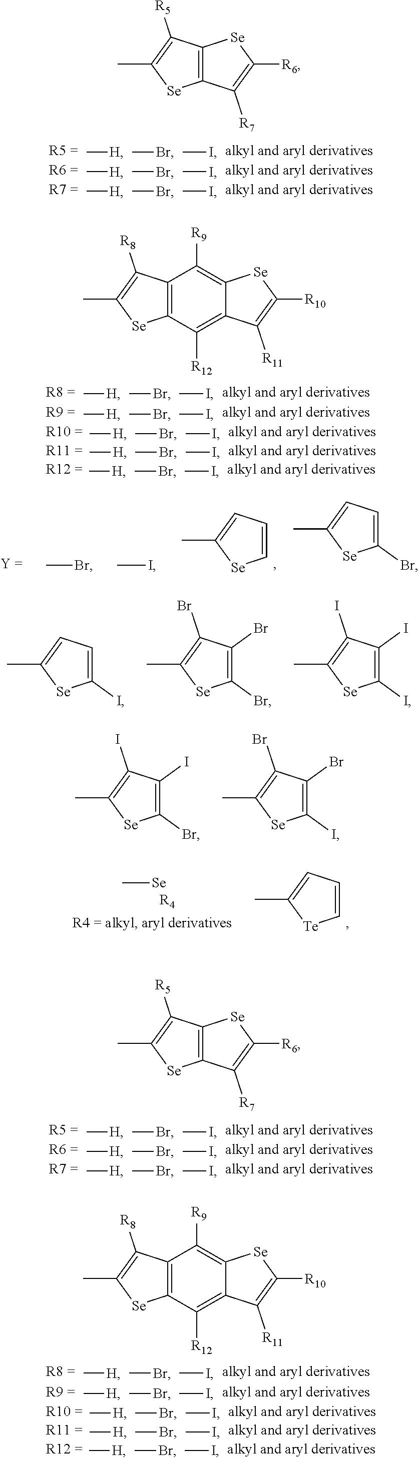

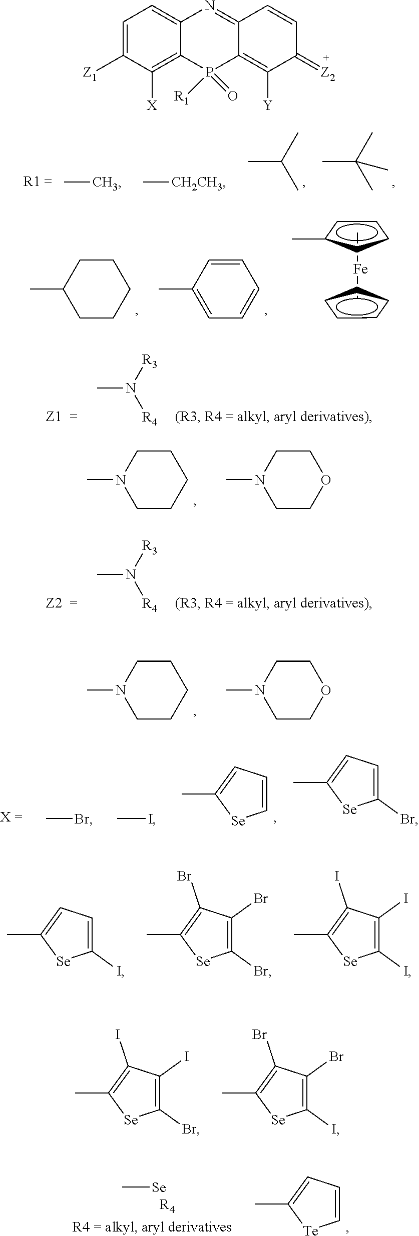

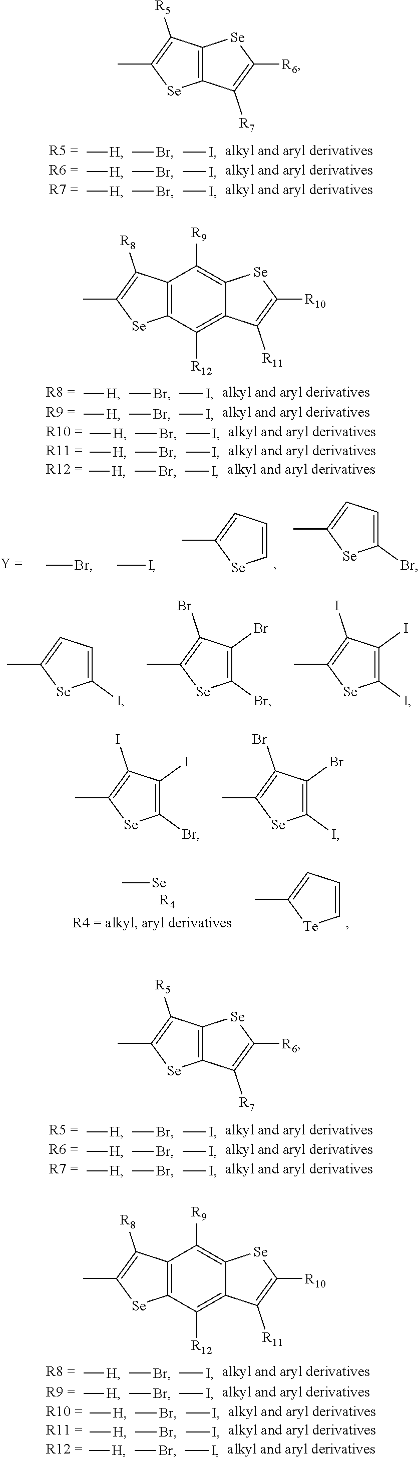

### Synthesized Compounds for Use in Photodynamic Therapy of Cancer:

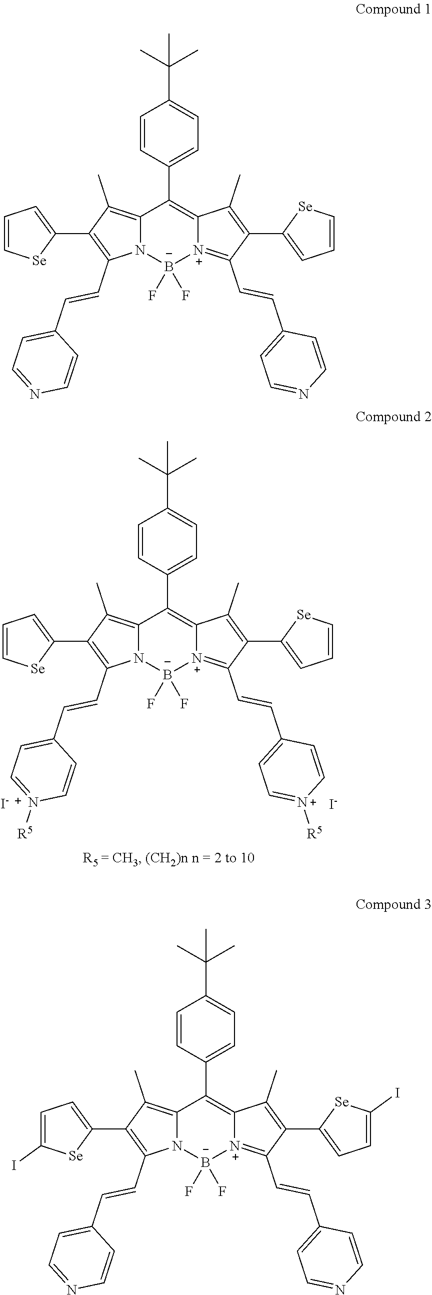

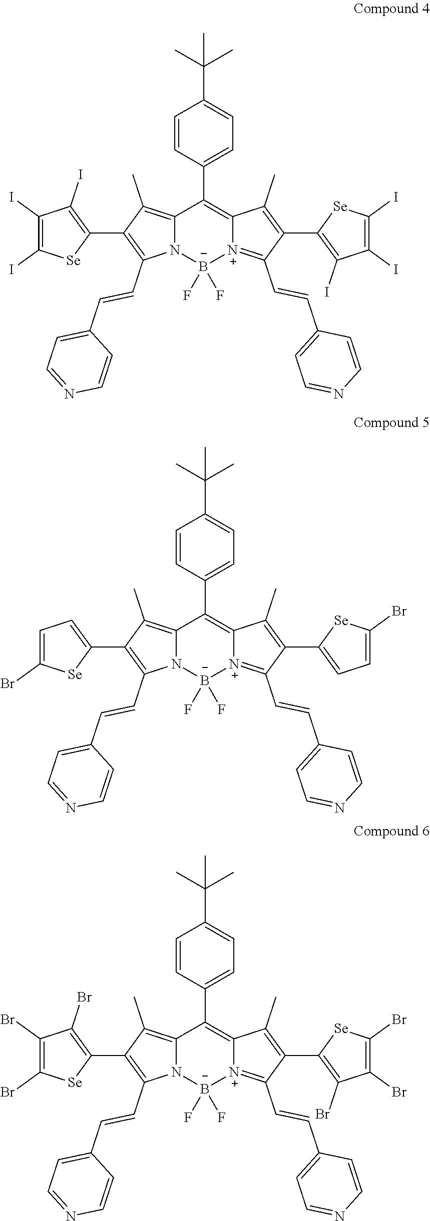 [SMILES: CC1=C(c2[se]c(I)c(I)c2I)C(/C=C/c2ccncc2)=[N+]2C1=C(c1ccc(C(C)(C)C)cc1)c1c(C)c(-c3[se]c(I)c(I)c3I)c(/C=C/c3ccncc3)n1[B-]2(F)F ; CC1=C(c2ccc(Br)[se]2)C(/C=C/c2ccncc2)=[N+]2C1=C(c1ccc(C(C)(C)C)cc1)c1c(C)c(-c3ccc(Br)[se]3)c(/C=C/c3ccncc3)n1[B-]2(F)F ; CC1=C(c2[se]c(Br)c(Br)c2Br)C(/C=C/c2ccncc2)=[N+]2C1=C(c1ccc(C(C)(C)C)cc1)c1c(C)c(-c3[se]c(Br)c(Br)c3Br)c(/C=C/c3ccncc3)n1[B-]2(F)F]

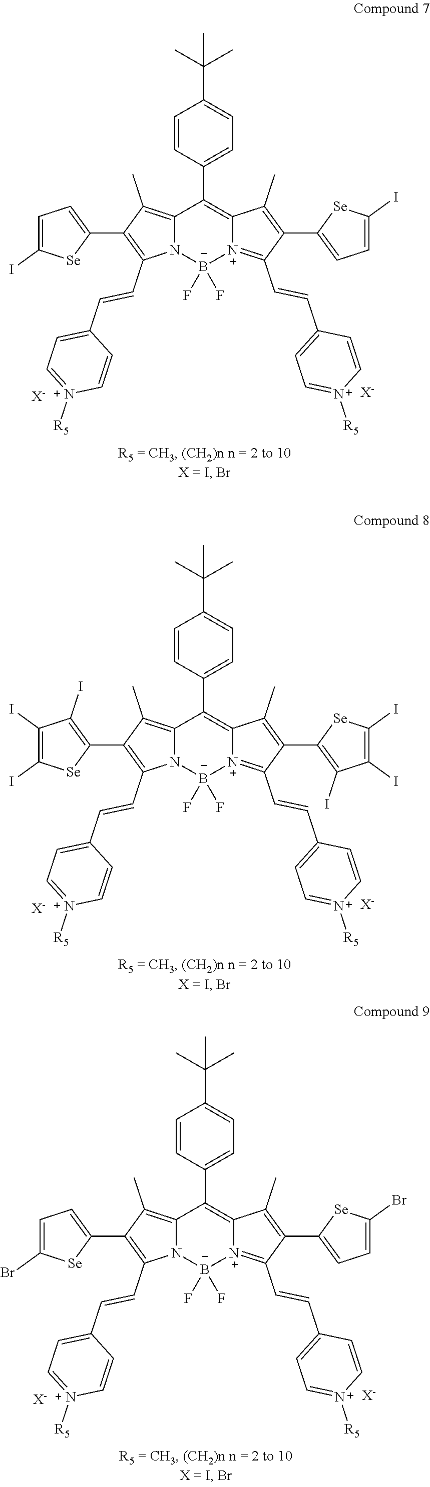

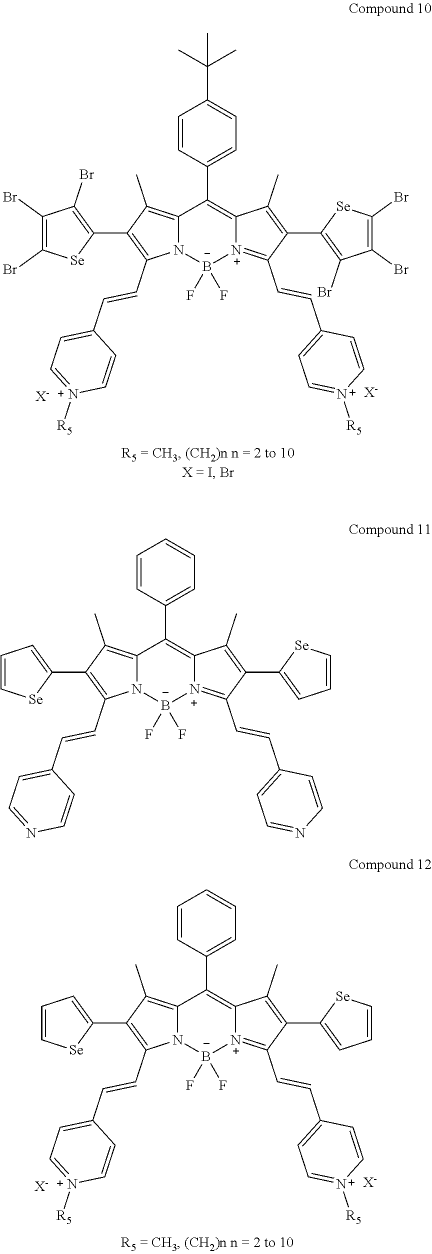

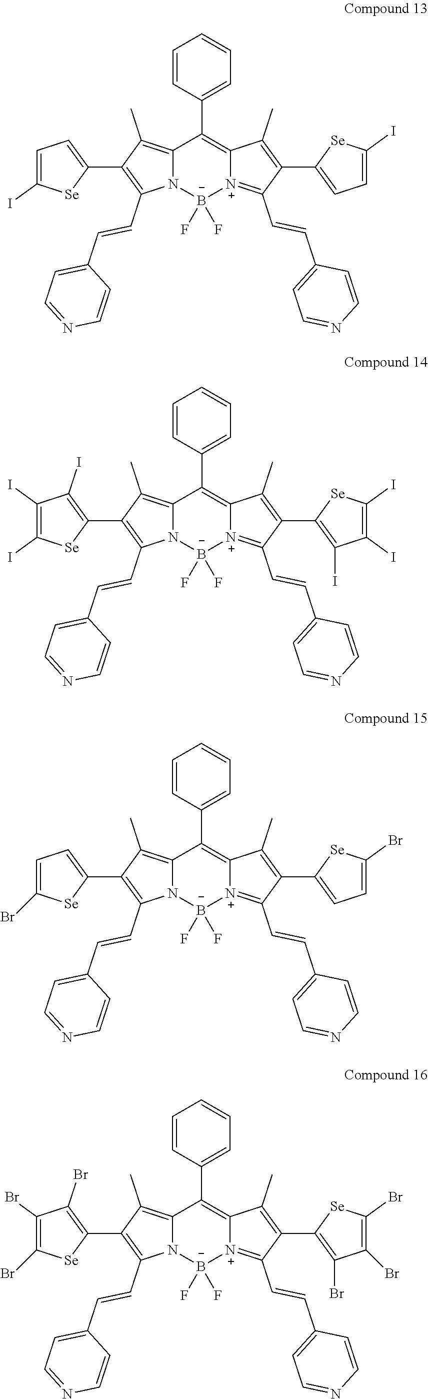 [SMILES: CC1=C(c2ccc(I)[se]2)C(/C=C/c2ccncc2)=[N+]2C1=C(c1ccccc1)c1c(C)c(-c3ccc(I)[se]3)c(/C=C/c3ccncc3)n1[B-]2(F)F ; CC1=C(c2[se]c(I)c(I)c2I)C(/C=C/c2ccncc2)=[N+]2C1=C(c1ccccc1)c1c(C)c(-c3[se]c(I)c(I)c3I)c(/C=C/c3ccncc3)n1[B-]2(F)F ; CC1=C(c2ccc(Br)[se]2)C(/C=C/c2ccncc2)=[N+]2C1=C(c1ccccc1)c1c(C)c(-c3ccc(Br)[se]3)c(/C=C/c3ccncc3)n1[B-]2(F)F ; CC1=C(c2[se]c(Br)c(Br)c2Br)C(/C=C/c2ccncc2)=[N+]2C1=C(c1ccccc1)c1c(C)c(-c3[se]c(Br)c(Br)c3Br)c(/C=C/c3ccncc3)n1[B-]2(F)F]

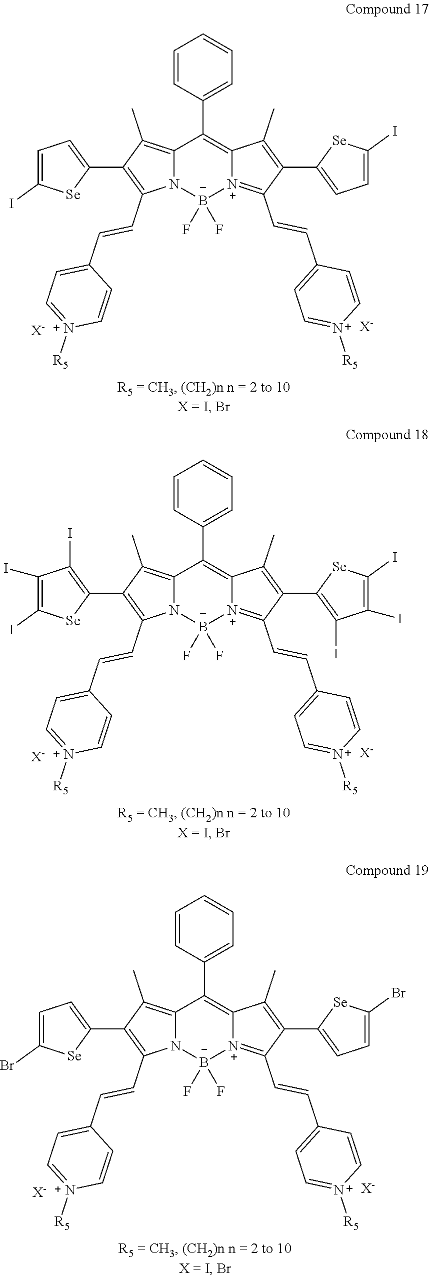

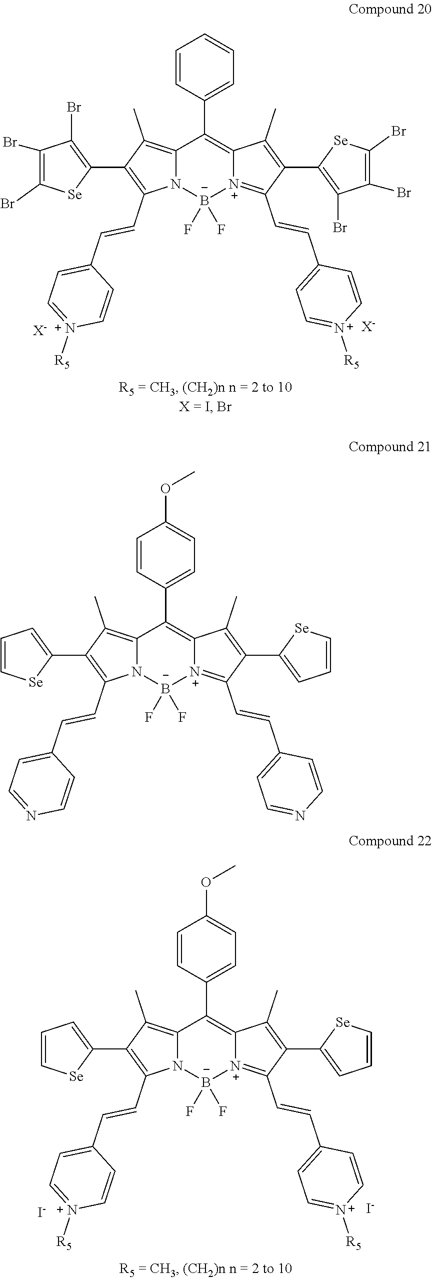

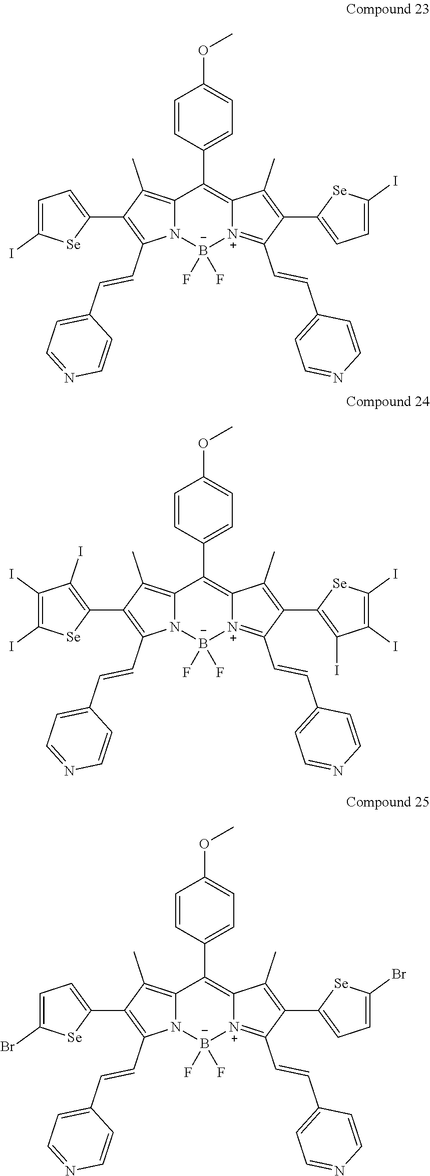 [SMILES: COc1ccc(C2=C3C(C)=C(c4ccc(I)[se]4)C(/C=C/c4ccncc4)=[N+]3[B-](F)(F)n3c(/C=C/c4ccncc4)c(-c4ccc(I)[se]4)c(C)c32)cc1 ; COc1ccc(C2=C3C(C)=C(c4[se]c(I)c(I)c4I)C(/C=C/c4ccncc4)=[N+]3[B-](F)(F)n3c(/C=C/c4ccncc4)c(-c4[se]c(I)c(I)c4I)c(C)c32)cc1 ; COc1ccc(C2=C3C(C)=C(c4ccc(Br)[se]4)C(/C=C/c4ccncc4)=[N+]3[B-](F)(F)n3c(/C=C/c4ccncc4)c(-c4ccc(Br)[se]4)c(C)c32)cc1]

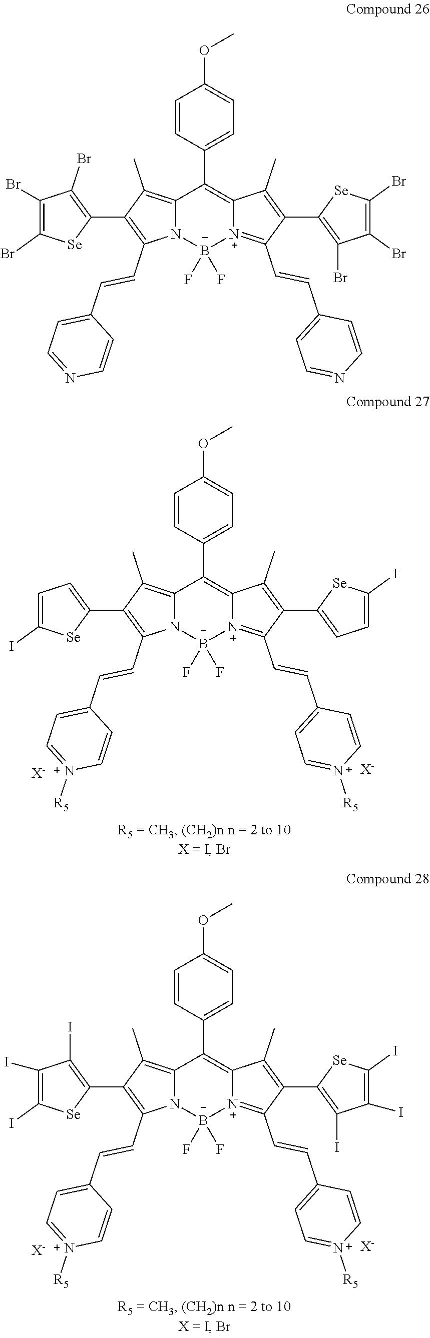

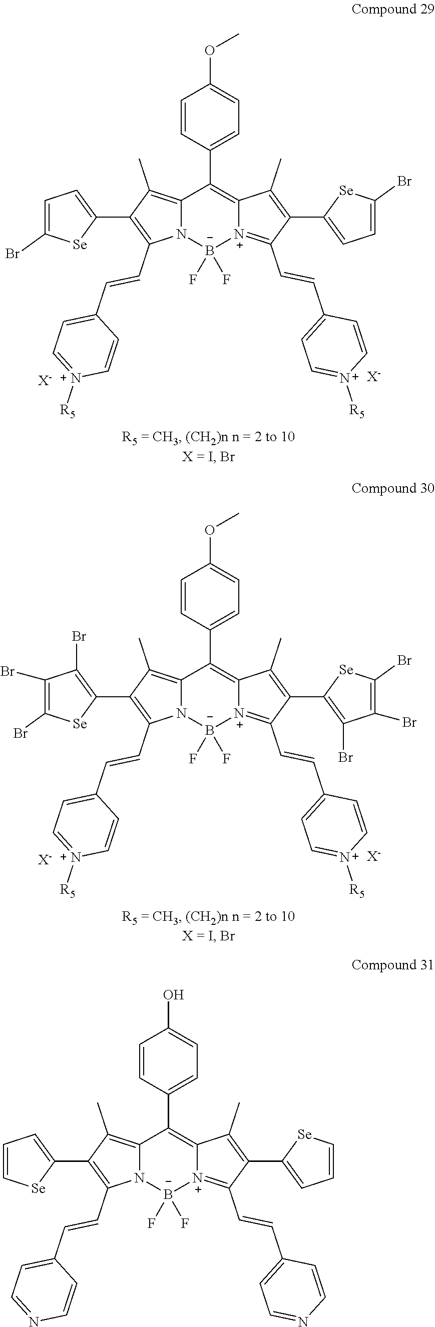

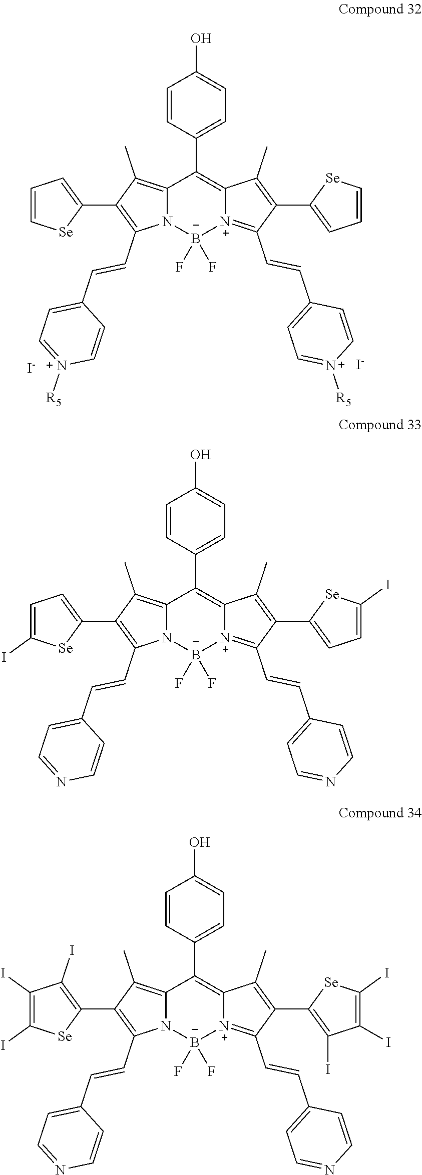

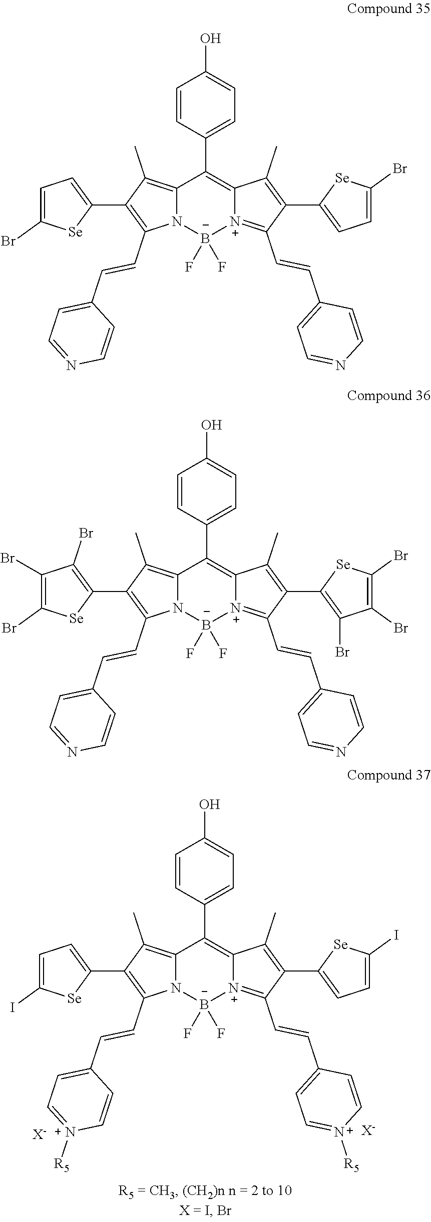

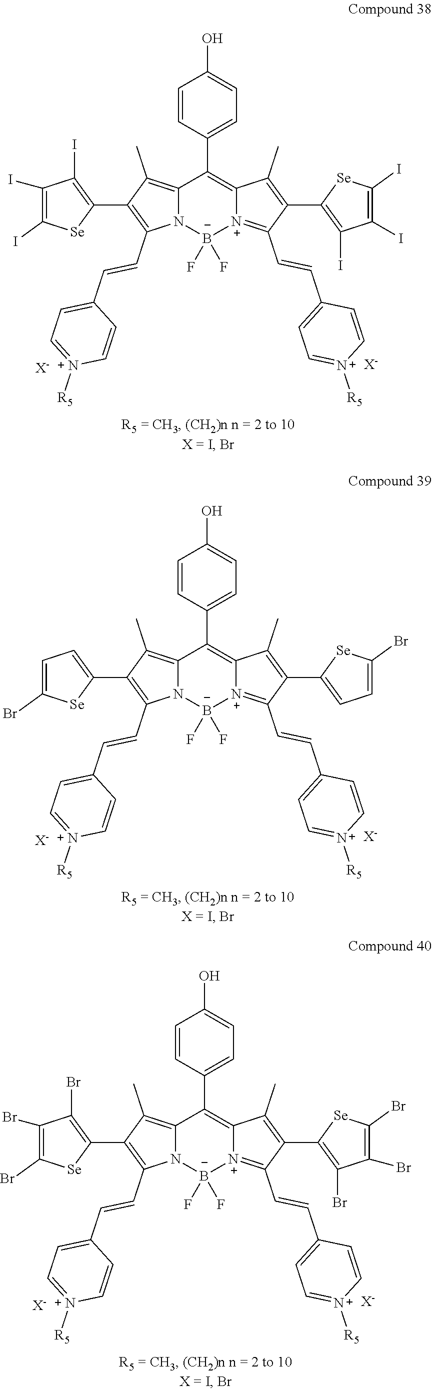

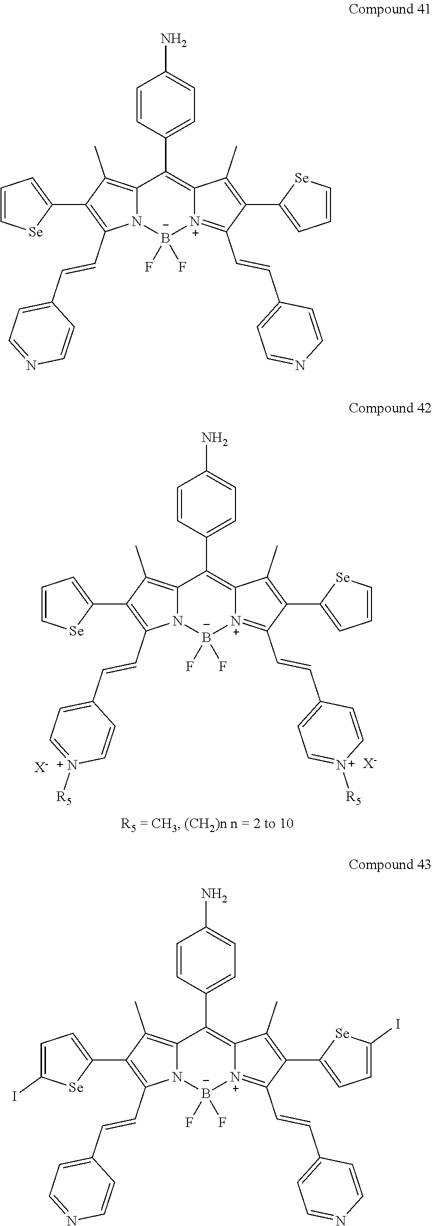

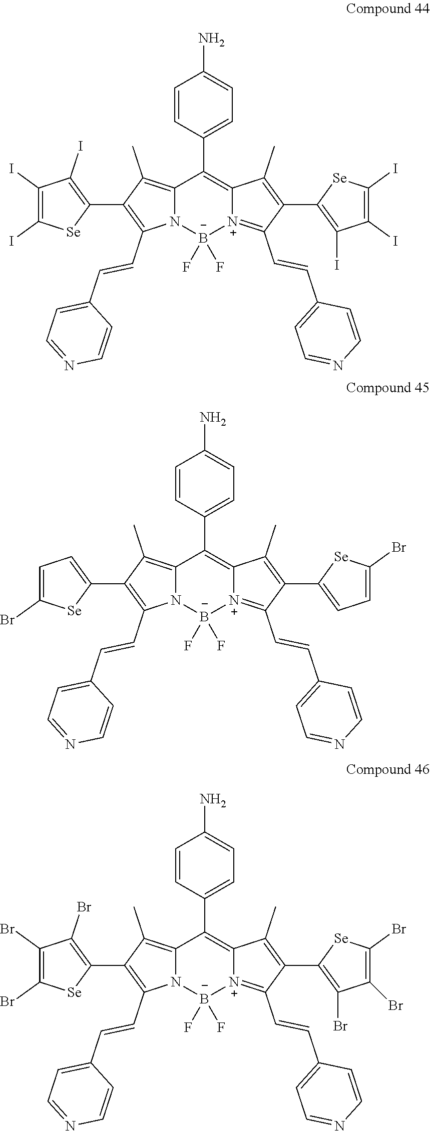 [SMILES: CC1=C(c2[se]c(I)c(I)c2I)C(/C=C/c2ccncc2)=[N+]2C1=C(c1ccc(N)cc1)c1c(C)c(-c3[se]c(I)c(I)c3I)c(/C=C/c3ccncc3)n1[B-]2(F)F ; CC1=C(c2ccc(Br)[se]2)C(/C=C/c2ccncc2)=[N+]2C1=C(c1ccc(N)cc1)c1c(C)c(-c3ccc(Br)[se]3)c(/C=C/c3ccncc3)n1[B-]2(F)F ; CC1=C(c2[se]c(Br)c(Br)c2Br)C(/C=C/c2ccncc2)=[N+]2C1=C(c1ccc(N)cc1)c1c(C)c(-c3[se]c(Br)c(Br)c3Br)c(/C=C/c3ccncc3)n1[B-]2(F)F]

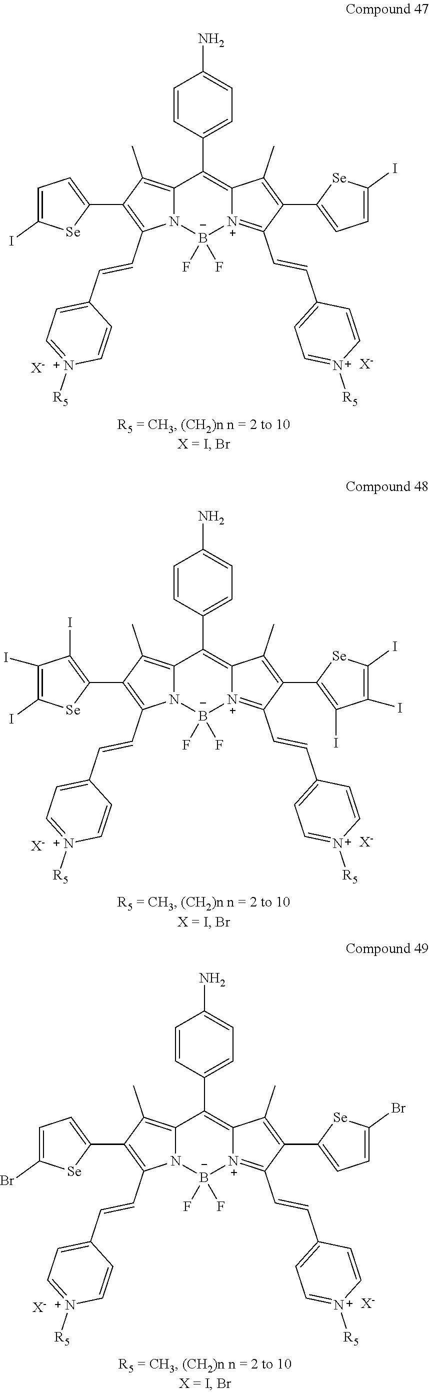

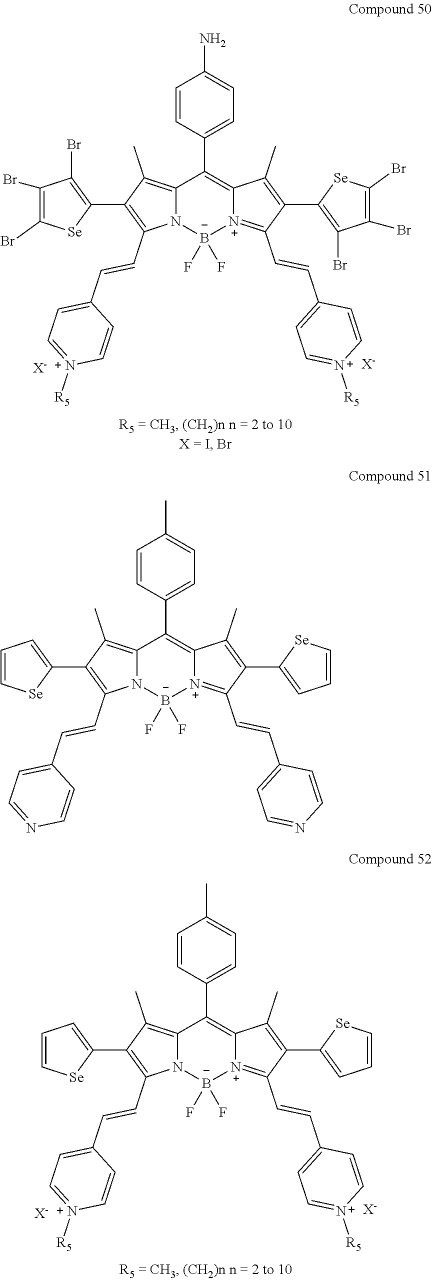

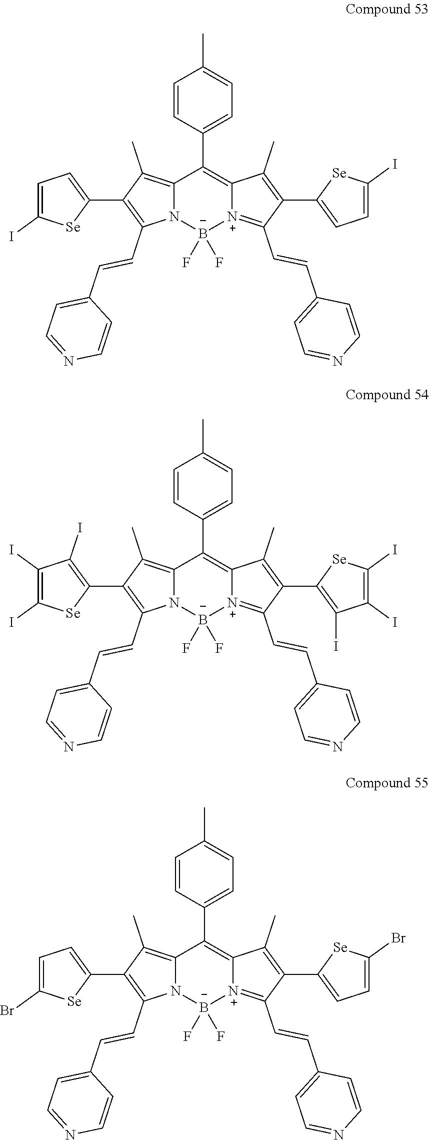 [SMILES: CC1=C(c2ccc(I)[se]2)C(/C=C/c2ccncc2)=[N+]2C1=C(c1ccc(C)cc1)c1c(C)c(-c3ccc(I)[se]3)c(/C=C/c3ccncc3)n1[B-]2(F)F ; CC1=C(c2[se]c(I)c(I)c2I)C(/C=C/c2ccncc2)=[N+]2C1=C(c1ccc(C)cc1)c1c(C)c(-c3[se]c(I)c(I)c3I)c(/C=C/c3ccncc3)n1[B-]2(F)F ; CC1=C(c2ccc(Br)[se]2)C(/C=C/c2ccncc2)=[N+]2C1=C(c1ccc(C)cc1)c1c(C)c(-c3ccc(Br)[se]3)c(/C=C/c3ccncc3)n1[B-]2(F)F]

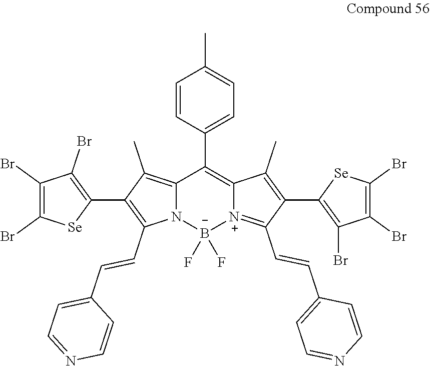 [SMILES: CC1=C(c2[se]c(Br)c(Br)c2Br)C(/C=C/c2ccncc2)=[N+]2C1=C(c1ccc(C)cc1)c1c(C)c(-c3[se]c(Br)c(Br)c3Br)c(/C=C/c3ccncc3)n1[B-]2(F)F]

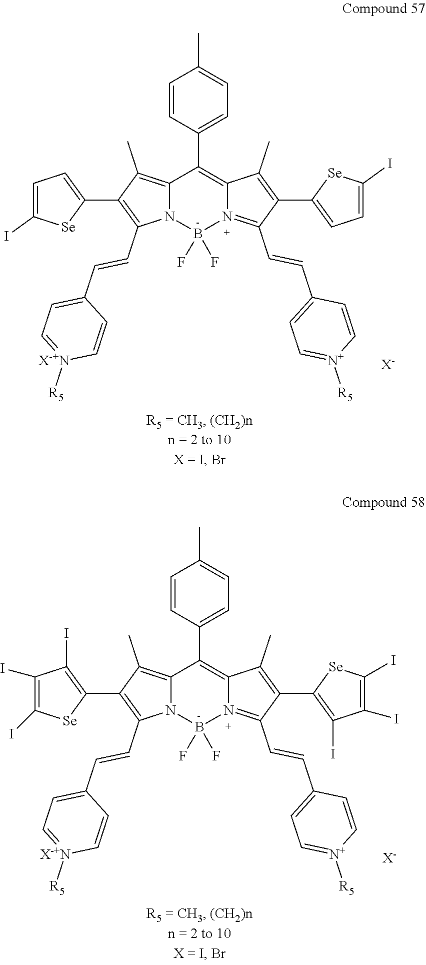

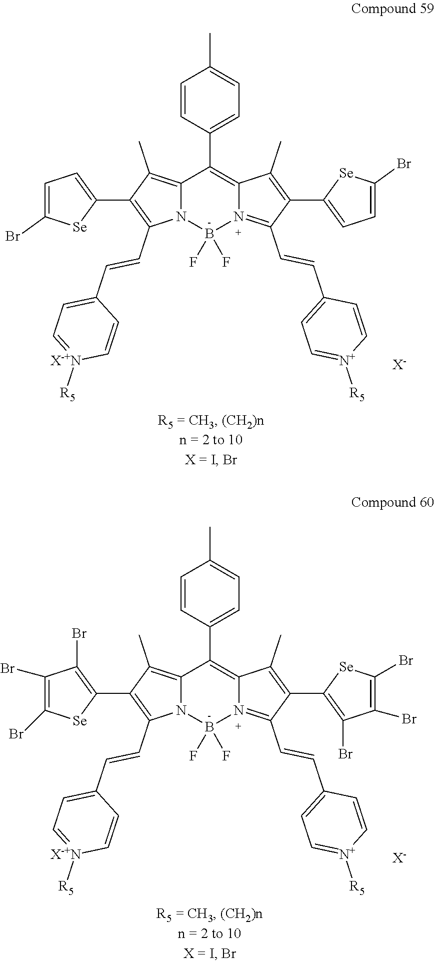

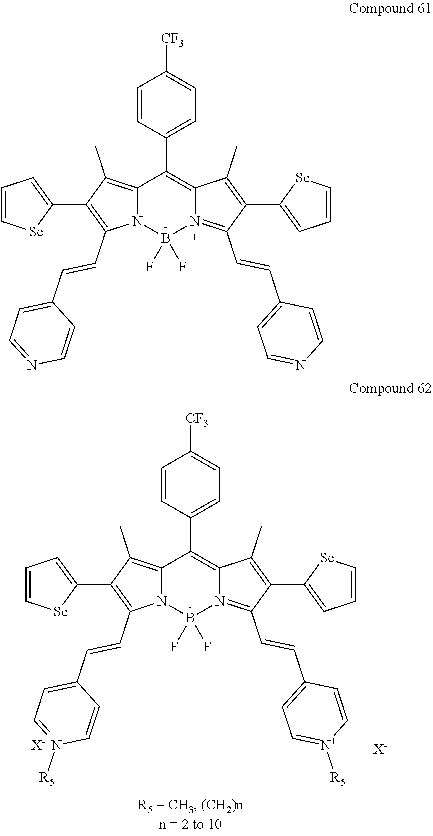

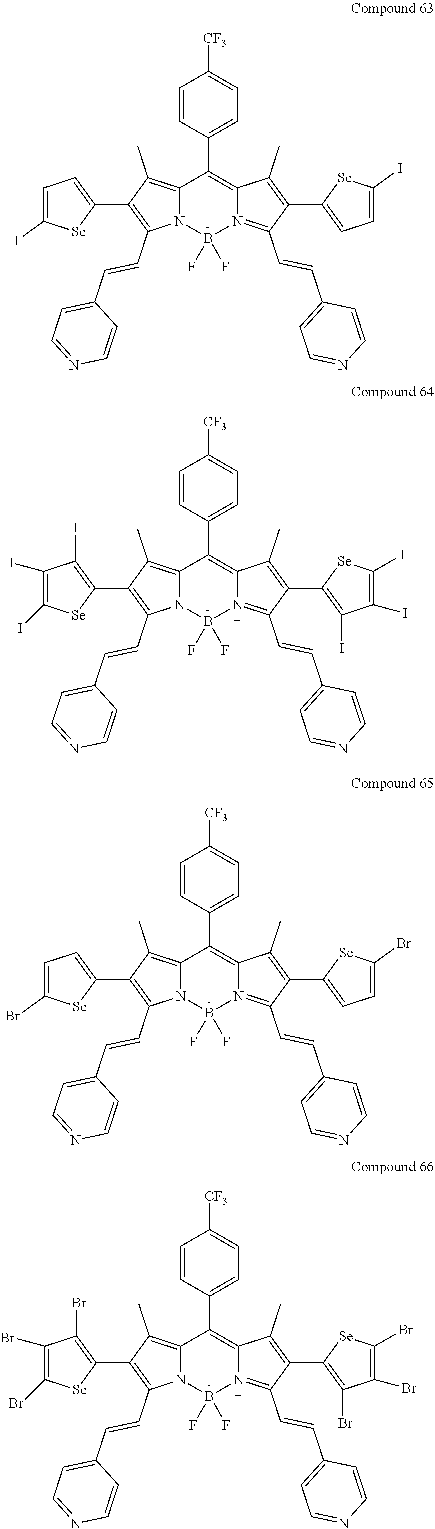

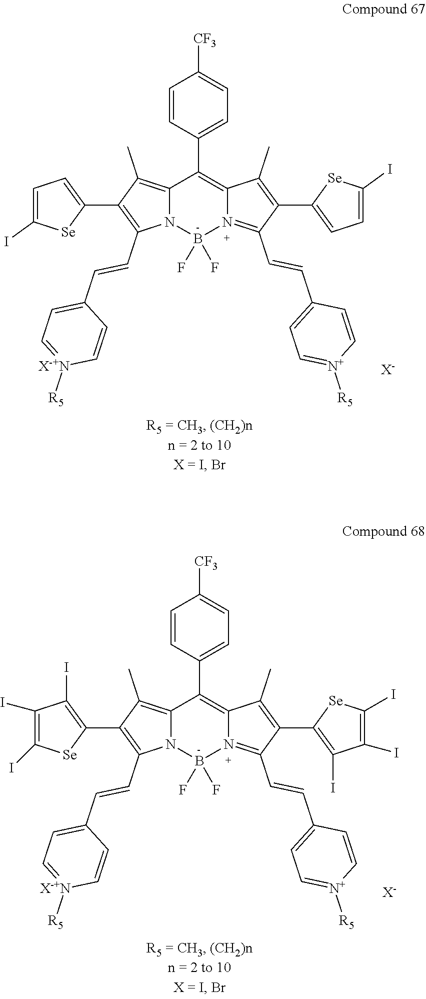

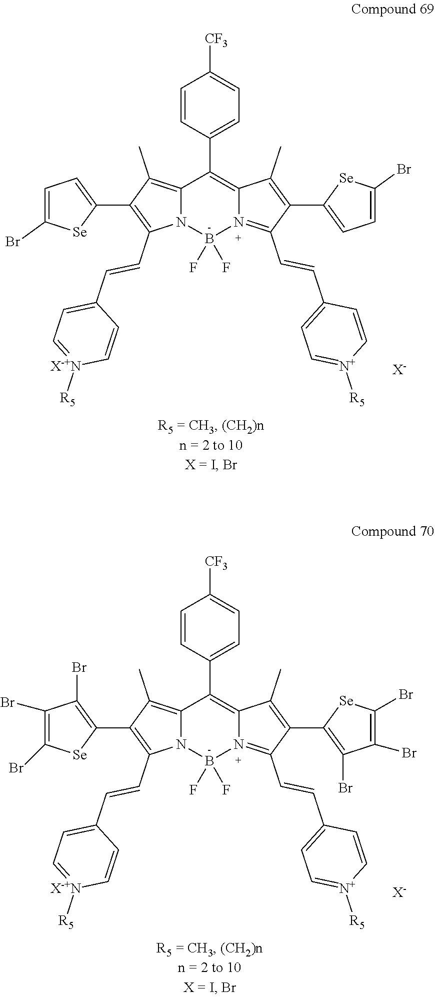

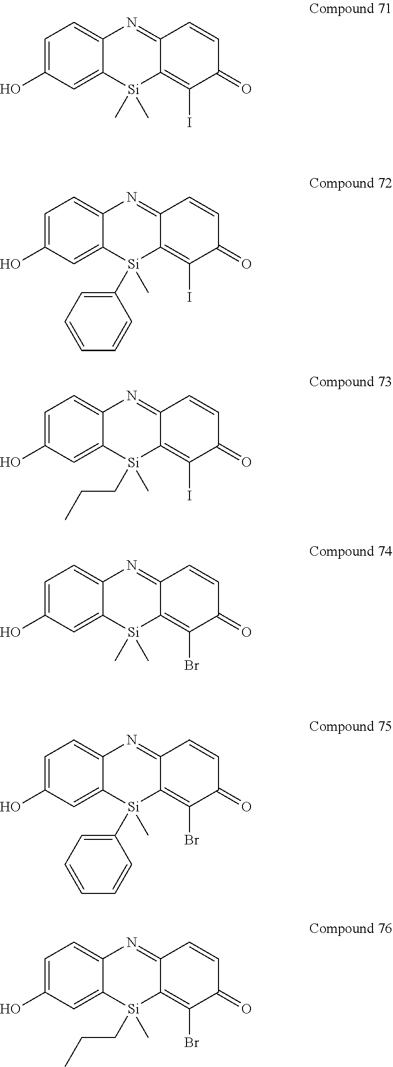

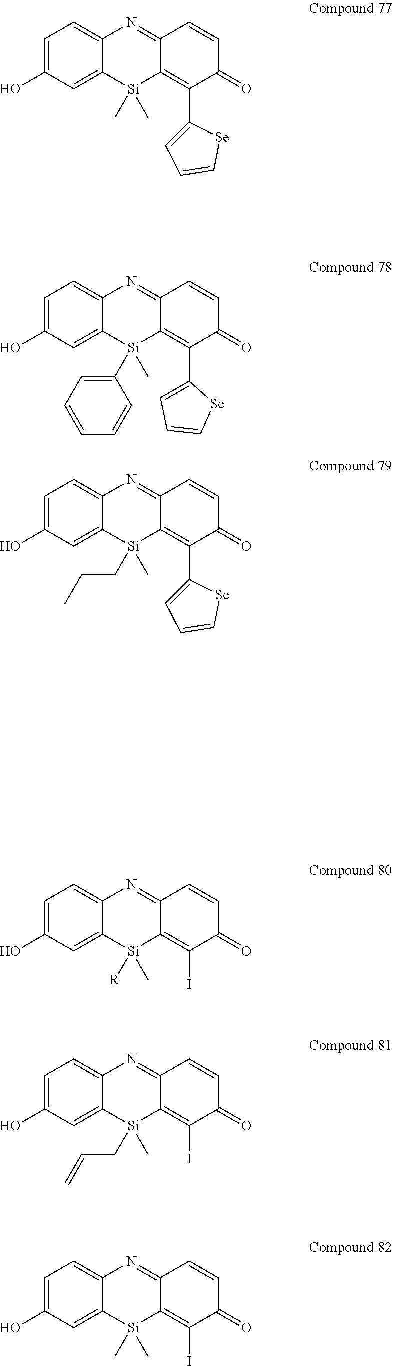

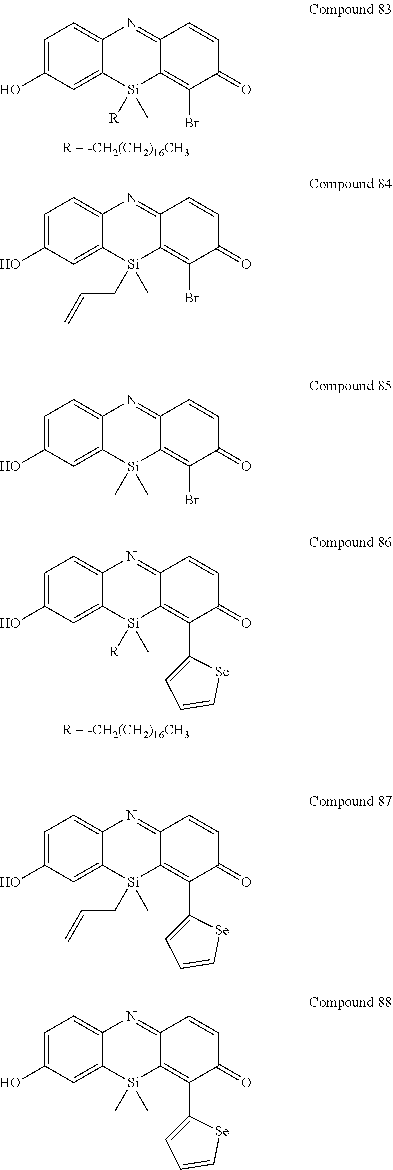

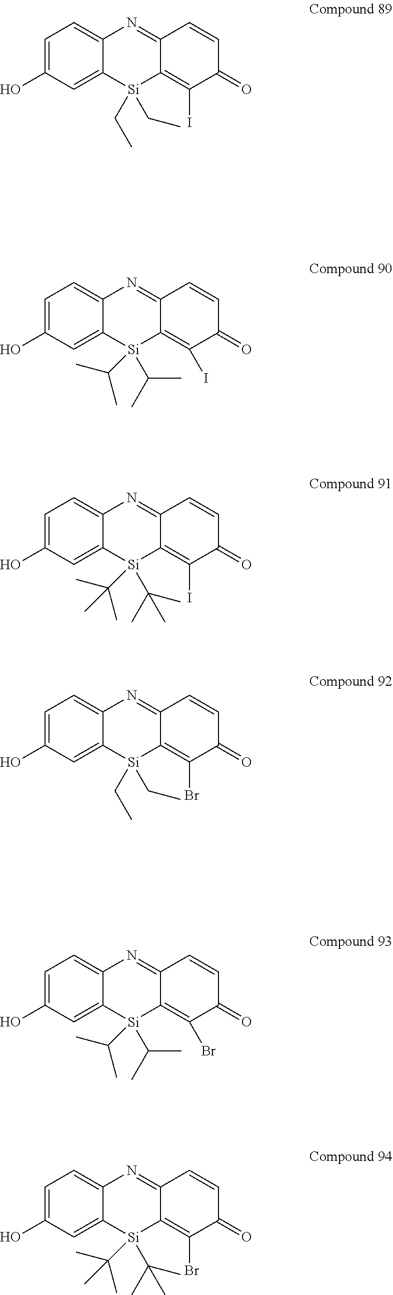 [SMILES: CC[Si]1(CC)C2=C(I)C(=O)C=CC2=Nc2ccc(O)cc21 ; CC(C)[Si]1(C(C)C)C2=C(I)C(=O)C=CC2=Nc2ccc(O)cc21 ; CC(C)(C)[Si]1(C(C)(C)C)C2=C(I)C(=O)C=CC2=Nc2ccc(O)cc21 ; CC[Si]1(CC)C2=C(Br)C(=O)C=CC2=Nc2ccc(O)cc21 ; CC(C)[Si]1(C(C)C)C2=C(Br)C(=O)C=CC2=Nc2ccc(O)cc21 ; CC(C)(C)[Si]1(C(C)(C)C)C2=C(Br)C(=O)C=CC2=Nc2ccc(O)cc21]

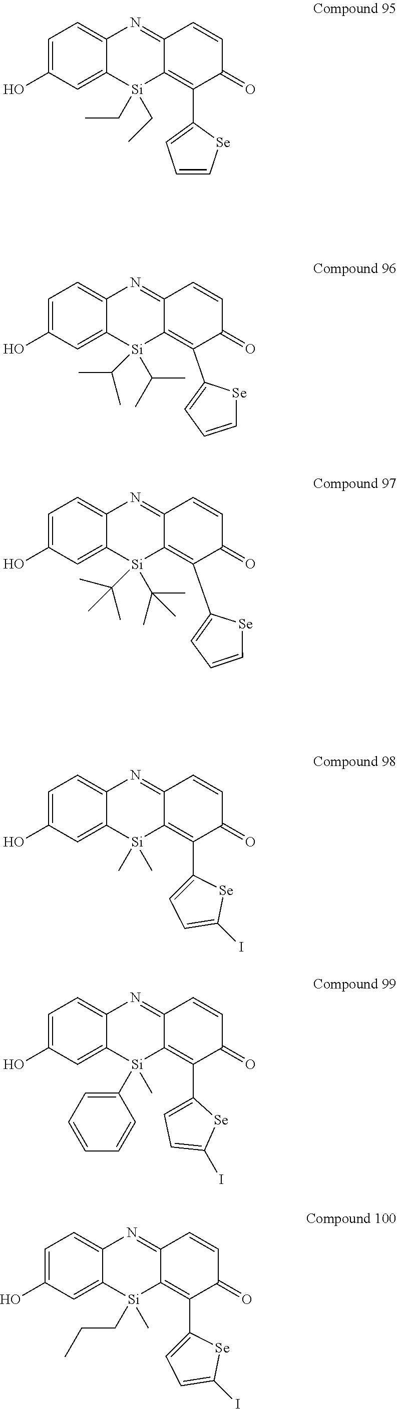 [SMILES: CC[Si]1(CC)C2=C(c3ccc[se]3)C(=O)C=CC2=Nc2ccc(O)cc21 ; CC(C)[Si]1(C(C)C)C2=C(c3ccc[se]3)C(=O)C=CC2=Nc2ccc(O)cc21 ; CC(C)(C)[Si]1(C(C)(C)C)C2=C(c3ccc[se]3)C(=O)C=CC2=Nc2ccc(O)cc21 ; C[Si]1(C)C2=C(c3ccc(I)[se]3)C(=O)C=CC2=Nc2ccc(O)cc21 ; C[Si]1(c2ccccc2)C2=C(c3ccc(I)[se]3)C(=O)C=CC2=Nc2ccc(O)cc21 ; CCC[Si]1(C)C2=C(c3ccc(I)[se]3)C(=O)C=CC2=Nc2ccc(O)cc21]

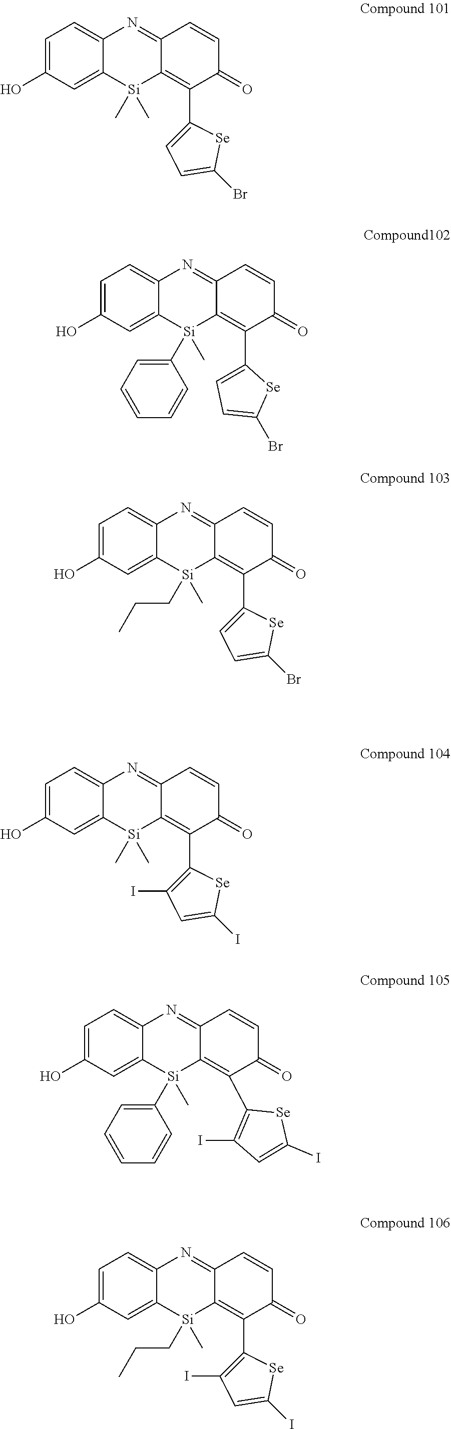 [SMILES: C[Si]1(C)C2=C(c3ccc(Br)[se]3)C(=O)C=CC2=Nc2ccc(O)cc21 ; C[Si]1(c2ccccc2)C2=C(c3ccc(Br)[se]3)C(=O)C=CC2=Nc2ccc(O)cc21 ; CCC[Si]1(C)C2=C(c3ccc(Br)[se]3)C(=O)C=CC2=Nc2ccc(O)cc21 ; C[Si]1(C)C2=C(c3[se]c(I)cc3I)C(=O)C=CC2=Nc2ccc(O)cc21 ; C[Si]1(c2ccccc2)C2=C(c3[se]c(I)cc3I)C(=O)C=CC2=Nc2ccc(O)cc21 ; CCC[Si]1(C)C2=C(c3[se]c(I)cc3I)C(=O)C=CC2=Nc2ccc(O)cc21]

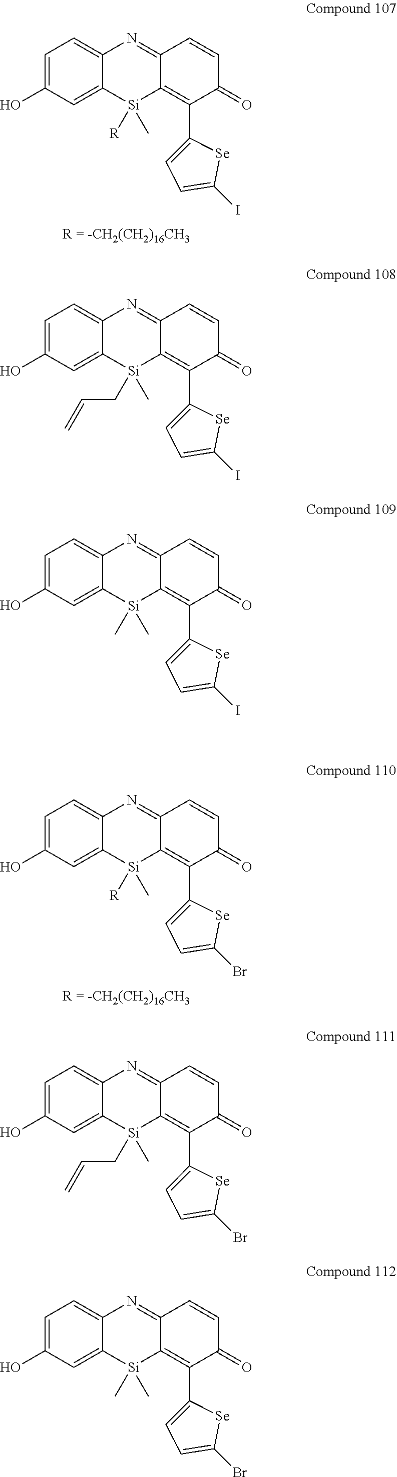

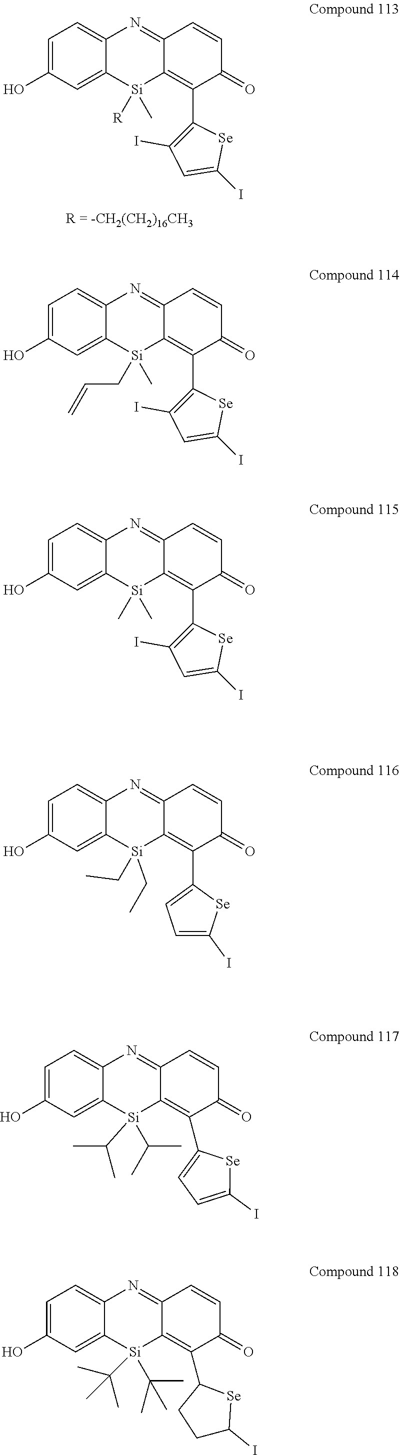

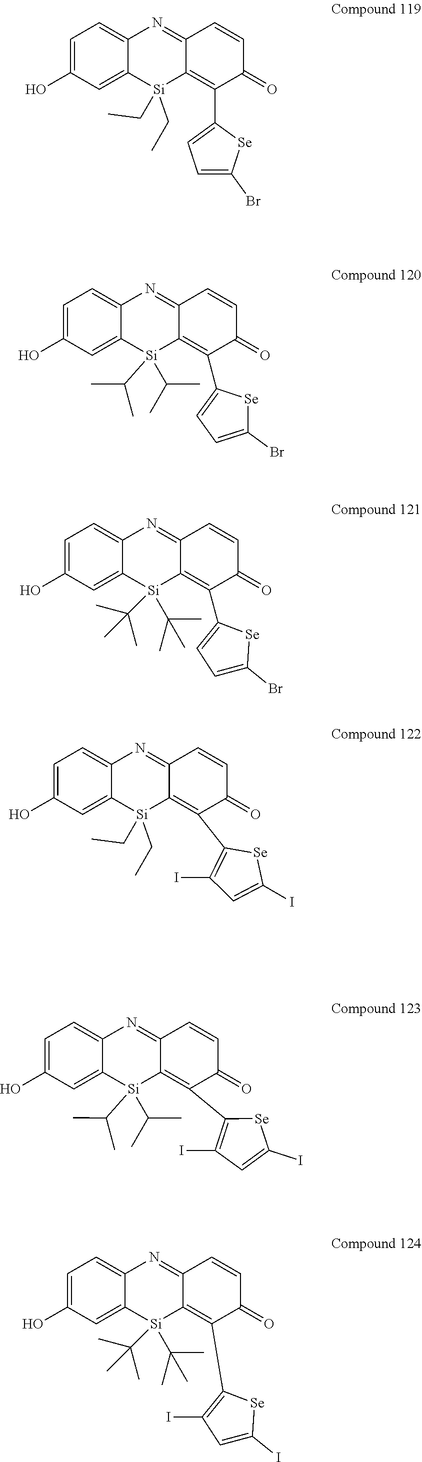 [SMILES: CC[Si]1(CC)C2=C(c3ccc(Br)[se]3)C(=O)C=CC2=Nc2ccc(O)cc21 ; CC(C)[Si]1(C(C)C)C2=C(c3ccc(Br)[se]3)C(=O)C=CC2=Nc2ccc(O)cc21 ; CC(C)(C)[Si]1(C(C)(C)C)C2=C(c3ccc(Br)[se]3)C(=O)C=CC2=Nc2ccc(O)cc21 ; CC[Si]1(CC)C2=C(c3[se]c(I)cc3I)C(=O)C=CC2=Nc2ccc(O)cc21 ; CC(C)[Si]1(C(C)C)C2=C(c3[se]c(I)cc3I)C(=O)C=CC2=Nc2ccc(O)cc21 ; CC(C)(C)[Si]1(C(C)(C)C)C2=C(c3[se]c(I)cc3I)C(=O)C=CC2=Nc2ccc(O)cc21]

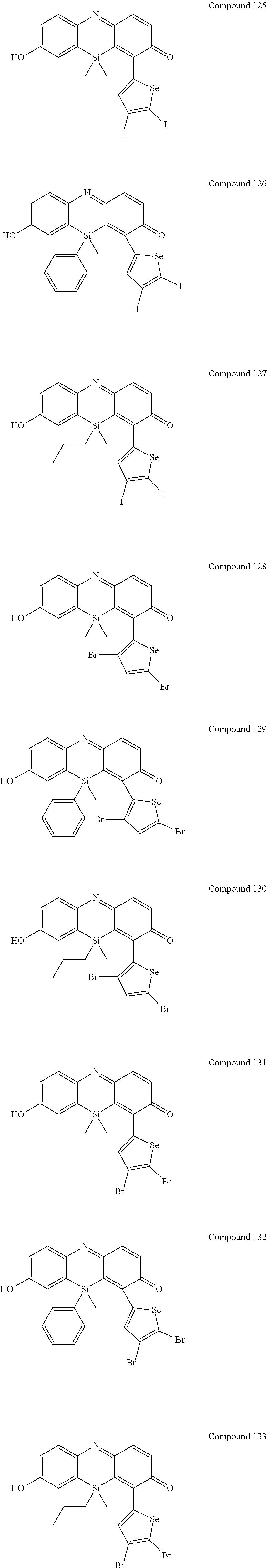

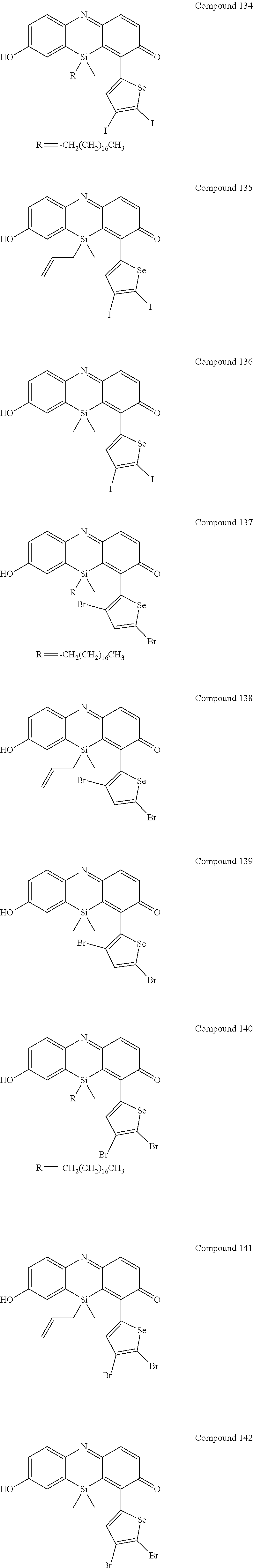

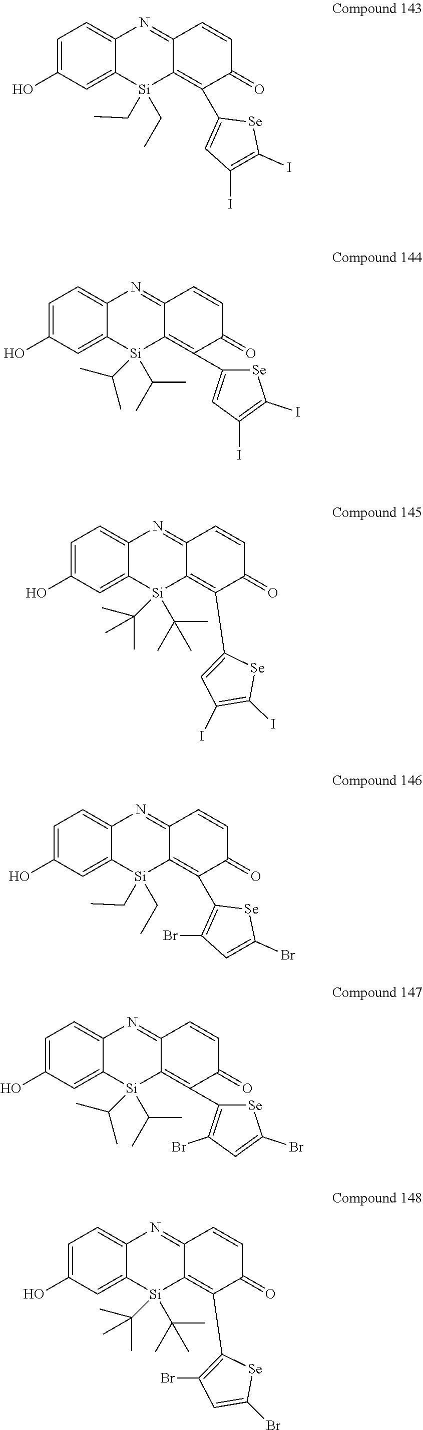 [SMILES: CC[Si]1(CC)C2=C(c3cc(I)c(I)[se]3)C(=O)C=CC2=Nc2ccc(O)cc21 ; CC(C)[Si]1(C(C)C)C2=C(c3cc(I)c(I)[se]3)C(=O)C=CC2=Nc2ccc(O)cc21 ; CC(C)(C)[Si]1(C(C)(C)C)C2=C(c3cc(I)c(I)[se]3)C(=O)C=CC2=Nc2ccc(O)cc21 ; CC[Si]1(CC)C2=C(c3[se]c(Br)cc3Br)C(=O)C=CC2=Nc2ccc(O)cc21 ; CC(C)[Si]1(C(C)C)C2=C(c3[se]c(Br)cc3Br)C(=O)C=CC2=Nc2ccc(O)cc21 ; CC(C)(C)[Si]1(C(C)(C)C)C2=C(c3[se]c(Br)cc3Br)C(=O)C=CC2=Nc2ccc(O)cc21]

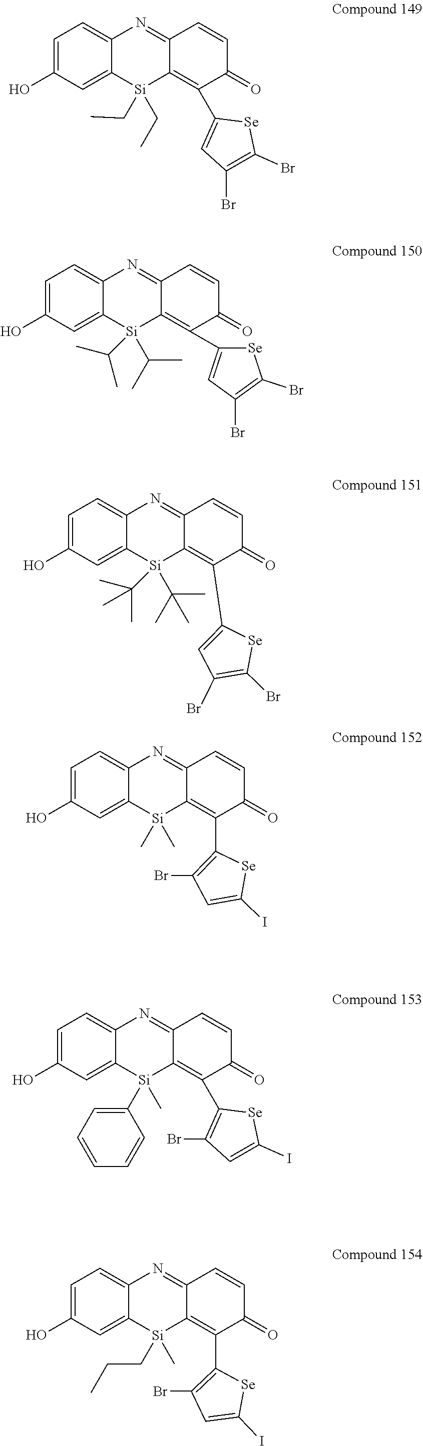

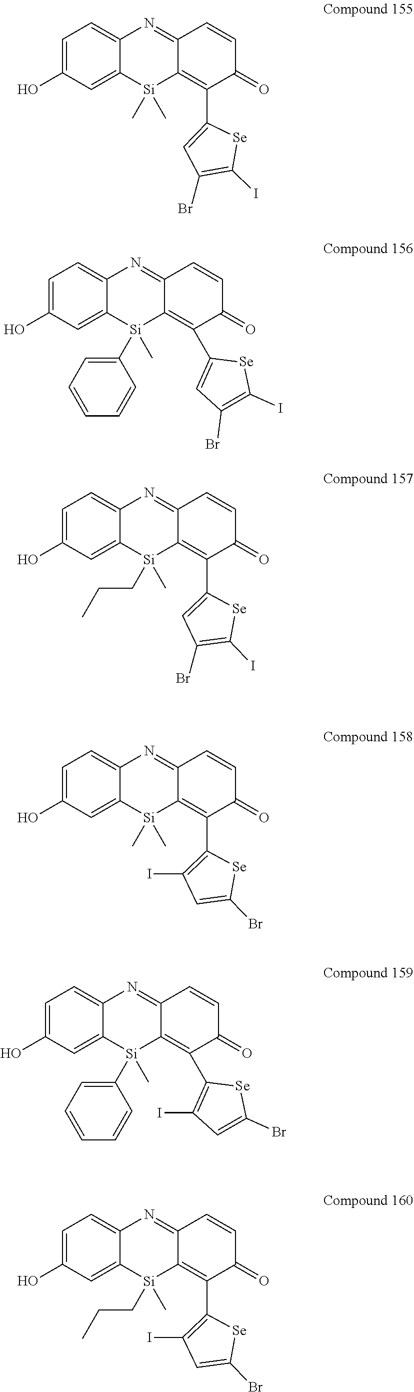 [SMILES: C[Si]1(C)C2=C(c3cc(Br)c(I)[se]3)C(=O)C=CC2=Nc2ccc(O)cc21 ; C[Si]1(c2ccccc2)C2=C(c3cc(Br)c(I)[se]3)C(=O)C=CC2=Nc2ccc(O)cc21 ; CCC[Si]1(C)C2=C(c3cc(Br)c(I)[se]3)C(=O)C=CC2=Nc2ccc(O)cc21 ; C[Si]1(C)C2=C(c3[se]c(Br)cc3I)C(=O)C=CC2=Nc2ccc(O)cc21 ; C[Si]1(c2ccccc2)C2=C(c3[se]c(Br)cc3I)C(=O)C=CC2=Nc2ccc(O)cc21 ; CCC[Si]1(C)C2=C(c3[se]c(Br)cc3I)C(=O)C=CC2=Nc2ccc(O)cc21]

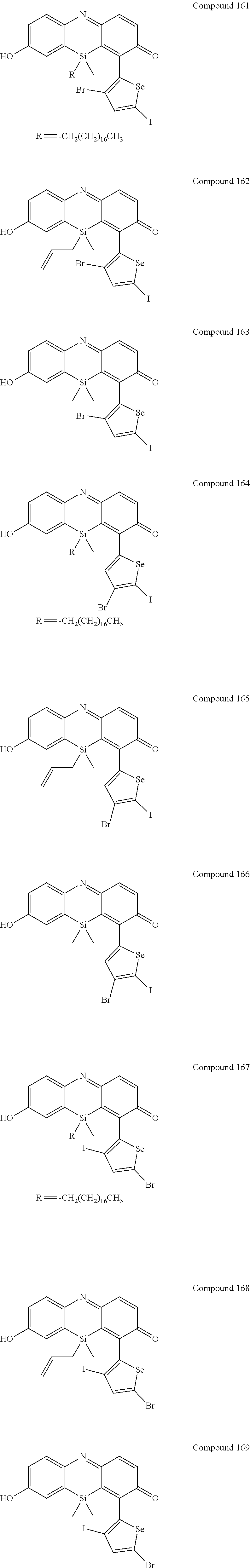

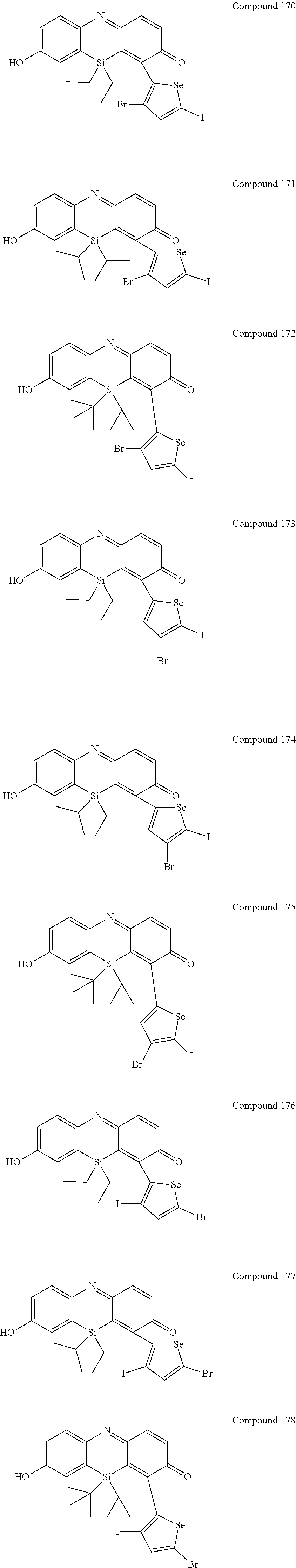

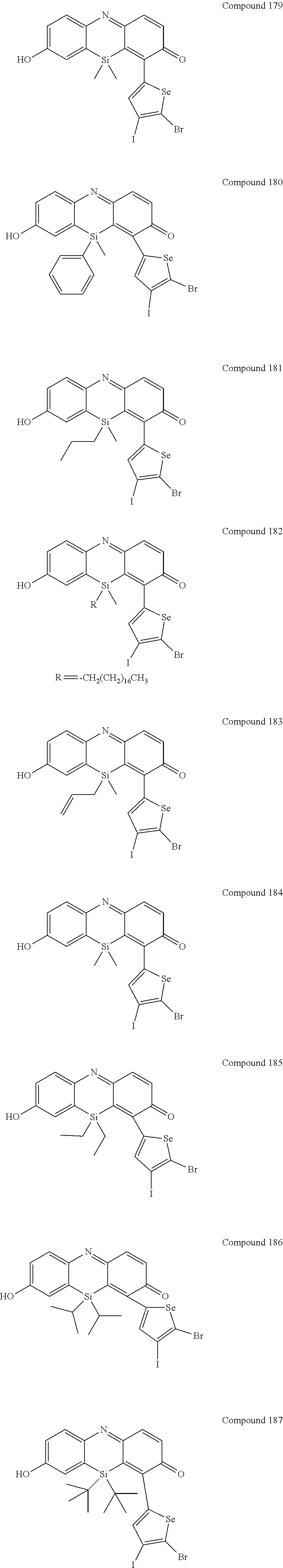

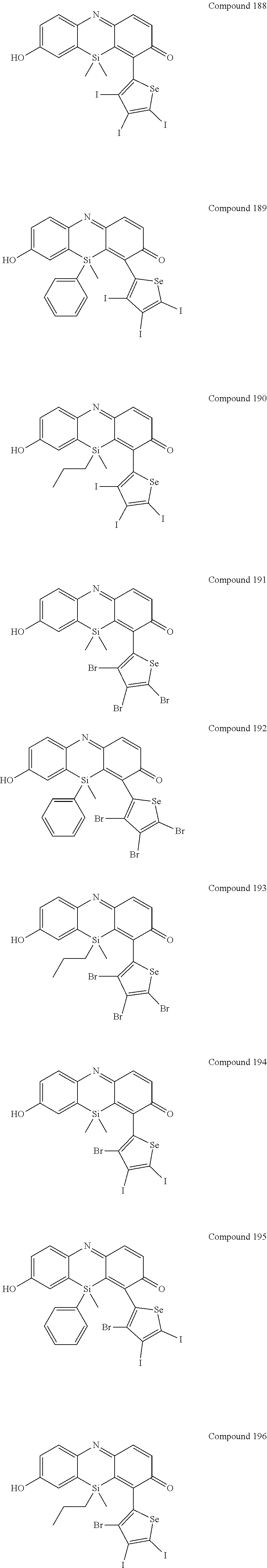

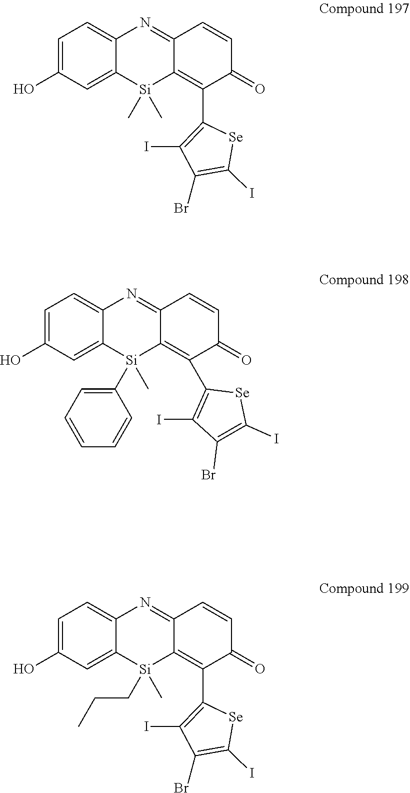 [SMILES: C[Si]1(C)C2=C(c3[se]c(I)c(Br)c3I)C(=O)C=CC2=Nc2ccc(O)cc21 ; C[Si]1(c2ccccc2)C2=C(c3[se]c(I)c(Br)c3I)C(=O)C=CC2=Nc2ccc(O)cc21 ; CCC[Si]1(C)C2=C(c3[se]c(I)c(Br)c3I)C(=O)C=CC2=Nc2ccc(O)cc21]

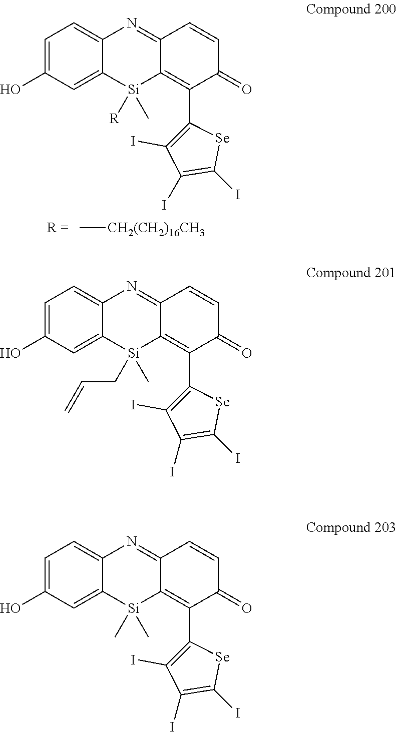

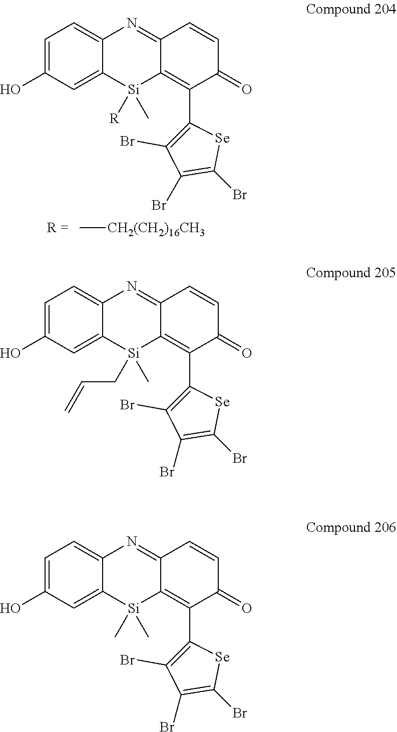

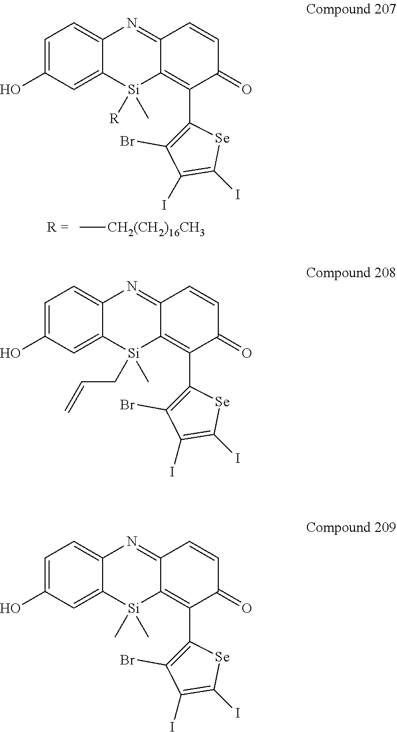

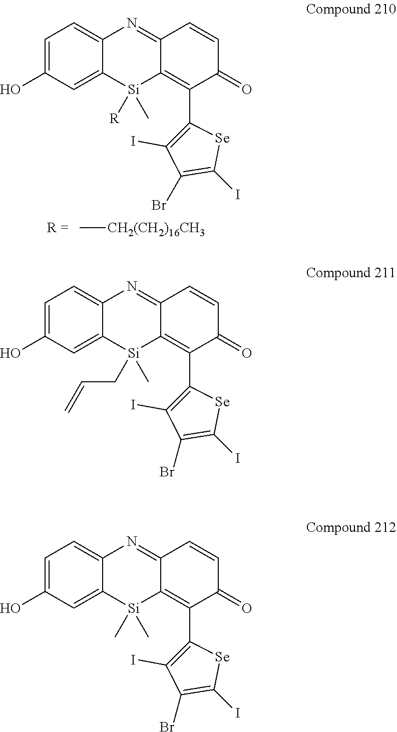

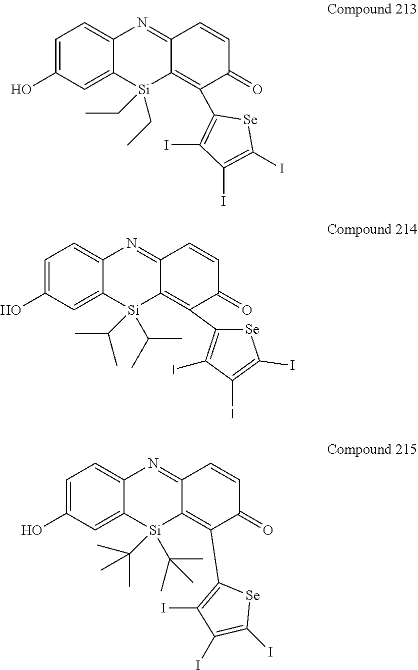 [SMILES: CC[Si]1(CC)C2=C(c3[se]c(I)c(I)c3I)C(=O)C=CC2=Nc2ccc(O)cc21 ; CC(C)[Si]1(C(C)C)C2=C(c3[se]c(I)c(I)c3I)C(=O)C=CC2=Nc2ccc(O)cc21 ; CC(C)(C)[Si]1(C(C)(C)C)C2=C(c3[se]c(I)c(I)c3I)C(=O)C=CC2=Nc2ccc(O)cc21]

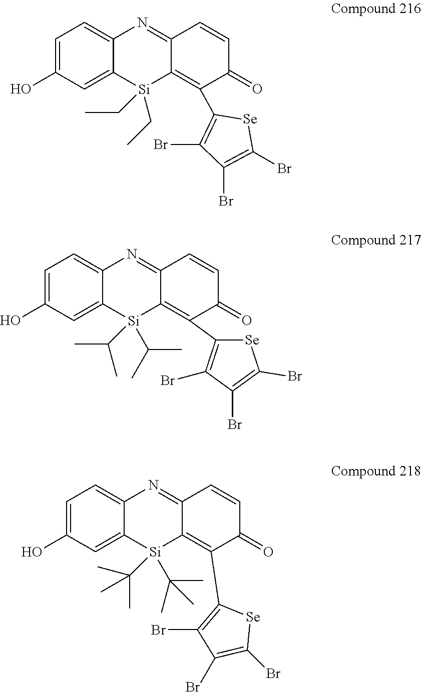 [SMILES: CC[Si]1(CC)C2=C(c3[se]c(Br)c(Br)c3Br)C(=O)C=CC2=Nc2ccc(O)cc21 ; CC(C)[Si]1(C(C)C)C2=C(c3[se]c(Br)c(Br)c3Br)C(=O)C=CC2=Nc2ccc(O)cc21 ; CC(C)(C)[Si]1(C(C)(C)C)C2=C(c3[se]c(Br)c(Br)c3Br)C(=O)C=CC2=Nc2ccc(O)cc21]

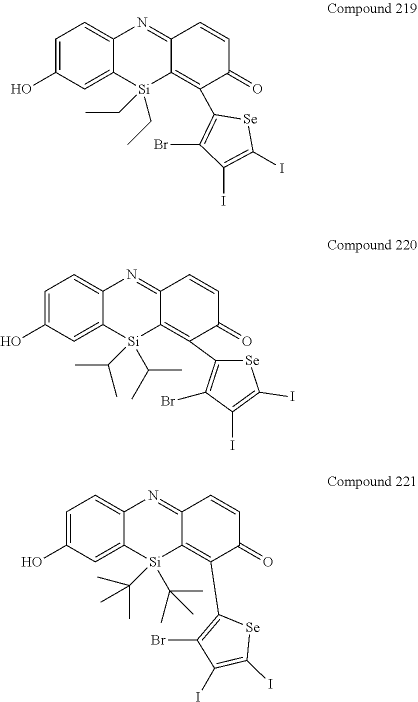 [SMILES: CC[Si]1(CC)C2=C(c3[se]c(I)c(I)c3Br)C(=O)C=CC2=Nc2ccc(O)cc21 ; CC(C)[Si]1(C(C)C)C2=C(c3[se]c(I)c(I)c3Br)C(=O)C=CC2=Nc2ccc(O)cc21 ; CC(C)(C)[Si]1(C(C)(C)C)C2=C(c3[se]c(I)c(I)c3Br)C(=O)C=CC2=Nc2ccc(O)cc21]

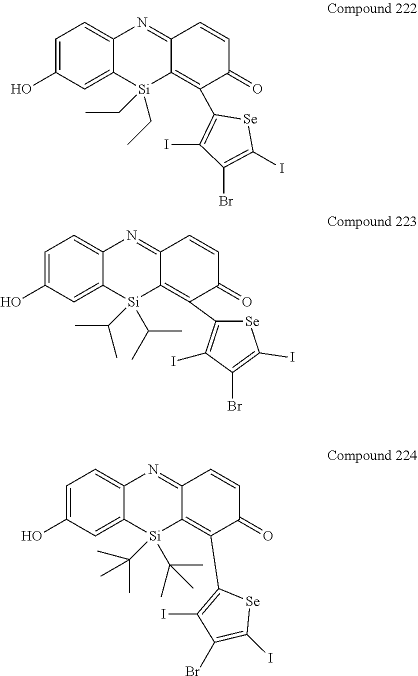 [SMILES: CC[Si]1(CC)C2=C(c3[se]c(I)c(Br)c3I)C(=O)C=CC2=Nc2ccc(O)cc21 ; CC(C)[Si]1(C(C)C)C2=C(c3[se]c(I)c(Br)c3I)C(=O)C=CC2=Nc2ccc(O)cc21 ; CC(C)(C)[Si]1(C(C)(C)C)C2=C(c3[se]c(I)c(Br)c3I)C(=O)C=CC2=Nc2ccc(O)cc21]

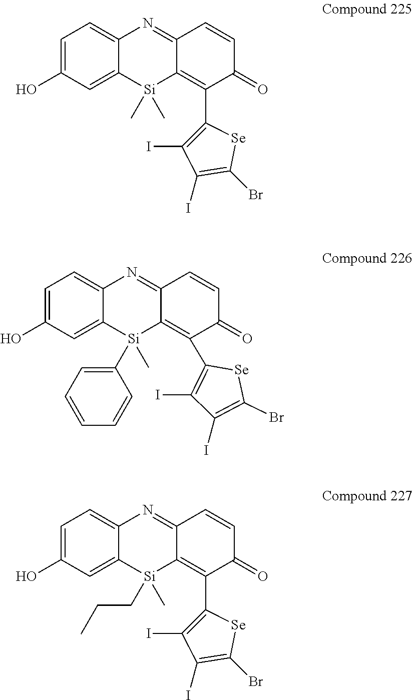 [SMILES: C[Si]1(C)C2=C(c3[se]c(Br)c(I)c3I)C(=O)C=CC2=Nc2ccc(O)cc21 ; C[Si]1(c2ccccc2)C2=C(c3[se]c(Br)c(I)c3I)C(=O)C=CC2=Nc2ccc(O)cc21 ; CCC[Si]1(C)C2=C(c3[se]c(Br)c(I)c3I)C(=O)C=CC2=Nc2ccc(O)cc21]

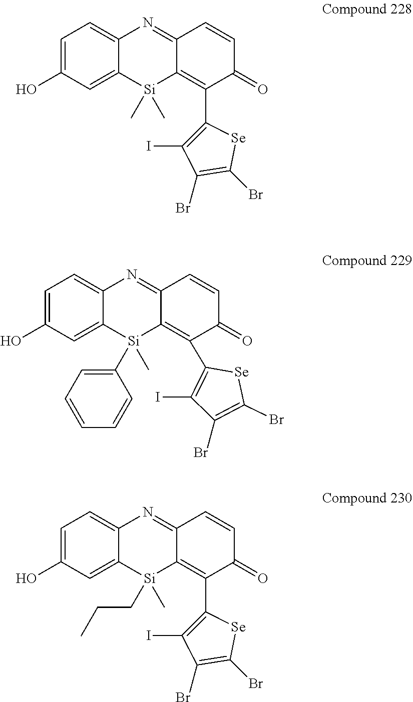 [SMILES: C[Si]1(C)C2=C(c3[se]c(Br)c(Br)c3I)C(=O)C=CC2=Nc2ccc(O)cc21 ; C[Si]1(c2ccccc2)C2=C(c3[se]c(Br)c(Br)c3I)C(=O)C=CC2=Nc2ccc(O)cc21 ; CCC[Si]1(C)C2=C(c3[se]c(Br)c(Br)c3I)C(=O)C=CC2=Nc2ccc(O)cc21]

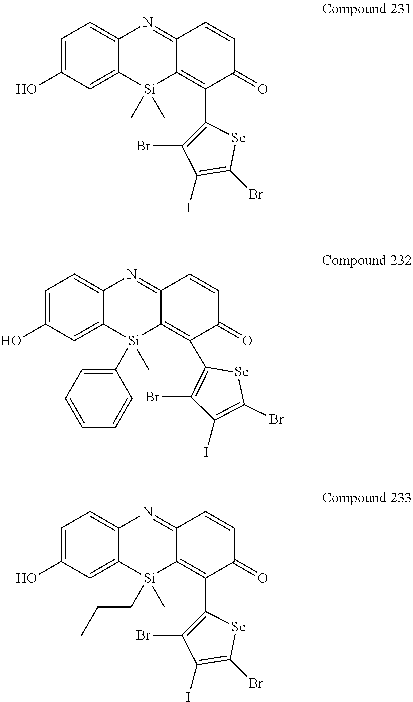 [SMILES: C[Si]1(C)C2=C(c3[se]c(Br)c(I)c3Br)C(=O)C=CC2=Nc2ccc(O)cc21 ; C[Si]1(c2ccccc2)C2=C(c3[se]c(Br)c(I)c3Br)C(=O)C=CC2=Nc2ccc(O)cc21 ; CCC[Si]1(C)C2=C(c3[se]c(Br)c(I)c3Br)C(=O)C=CC2=Nc2ccc(O)cc21]

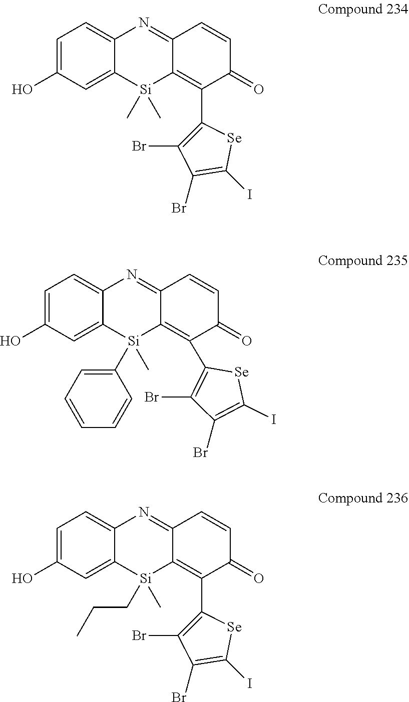 [SMILES: C[Si]1(C)C2=C(c3[se]c(I)c(Br)c3Br)C(=O)C=CC2=Nc2ccc(O)cc21 ; C[Si]1(c2ccccc2)C2=C(c3[se]c(I)c(Br)c3Br)C(=O)C=CC2=Nc2ccc(O)cc21 ; CCC[Si]1(C)C2=C(c3[se]c(I)c(Br)c3Br)C(=O)C=CC2=Nc2ccc(O)cc21]

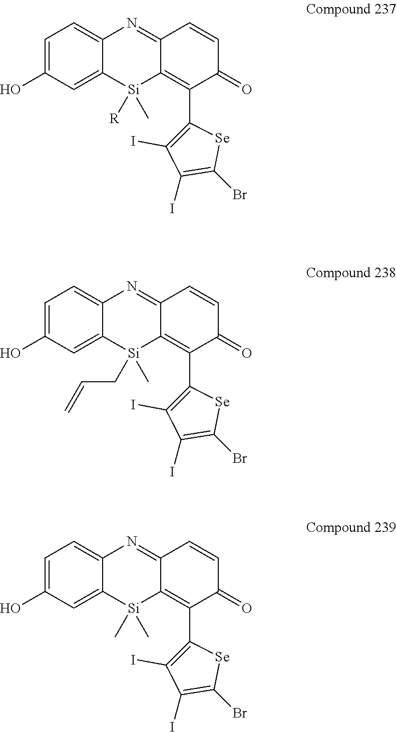

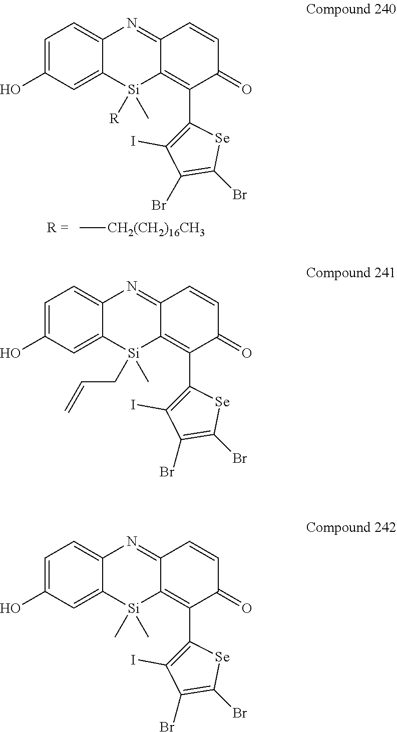

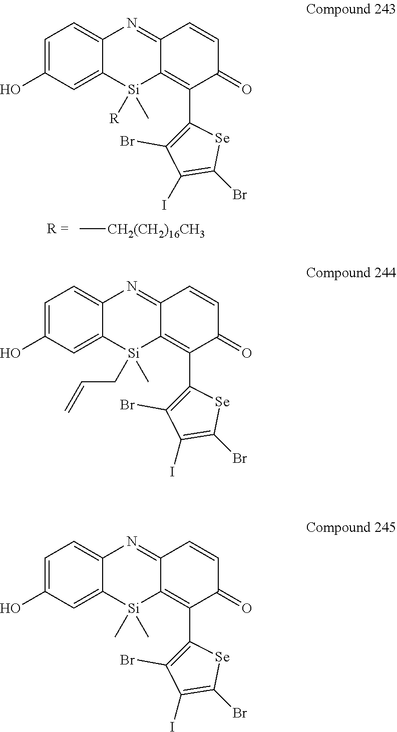

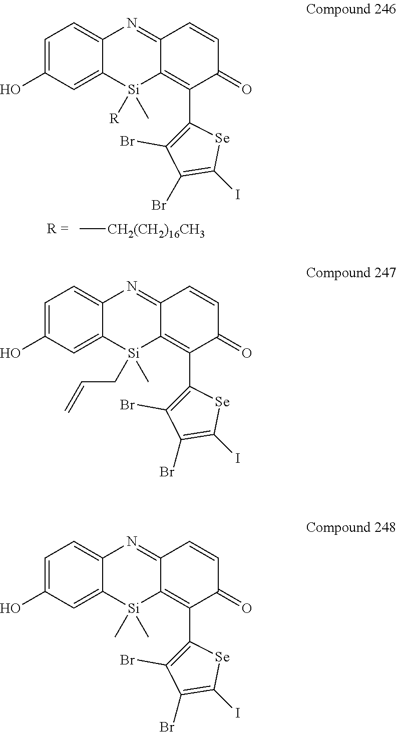

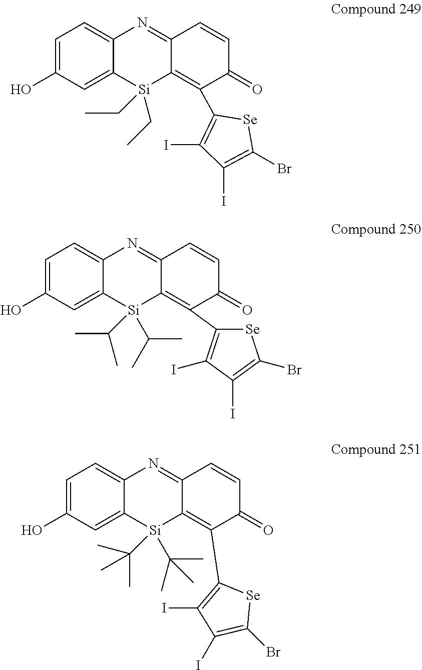 [SMILES: CC[Si]1(CC)C2=C(c3[se]c(Br)c(I)c3I)C(=O)C=CC2=Nc2ccc(O)cc21 ; CC(C)[Si]1(C(C)C)C2=C(c3[se]c(Br)c(I)c3I)C(=O)C=CC2=Nc2ccc(O)cc21 ; CC(C)(C)[Si]1(C(C)(C)C)C2=C(c3[se]c(Br)c(I)c3I)C(=O)C=CC2=Nc2ccc(O)cc21]

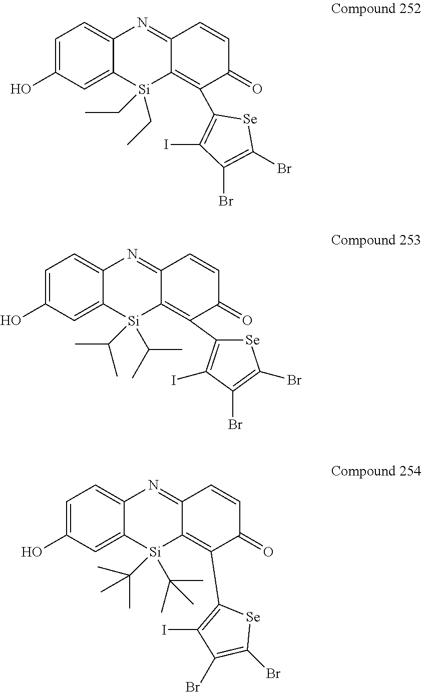 [SMILES: CC[Si]1(CC)C2=C(c3[se]c(Br)c(Br)c3I)C(=O)C=CC2=Nc2ccc(O)cc21 ; CC(C)[Si]1(C(C)C)C2=C(c3[se]c(Br)c(Br)c3I)C(=O)C=CC2=Nc2ccc(O)cc21 ; CC(C)(C)[Si]1(C(C)(C)C)C2=C(c3[se]c(Br)c(Br)c3I)C(=O)C=CC2=Nc2ccc(O)cc21]

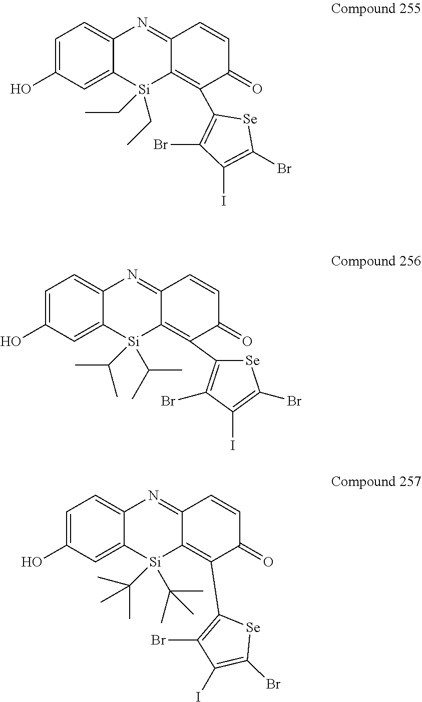 [SMILES: CC[Si]1(CC)C2=C(c3[se]c(Br)c(I)c3Br)C(=O)C=CC2=Nc2ccc(O)cc21 ; CC(C)[Si]1(C(C)C)C2=C(c3[se]c(Br)c(I)c3Br)C(=O)C=CC2=Nc2ccc(O)cc21 ; CC(C)(C)[Si]1(C(C)(C)C)C2=C(c3[se]c(Br)c(I)c3Br)C(=O)C=CC2=Nc2ccc(O)cc21]

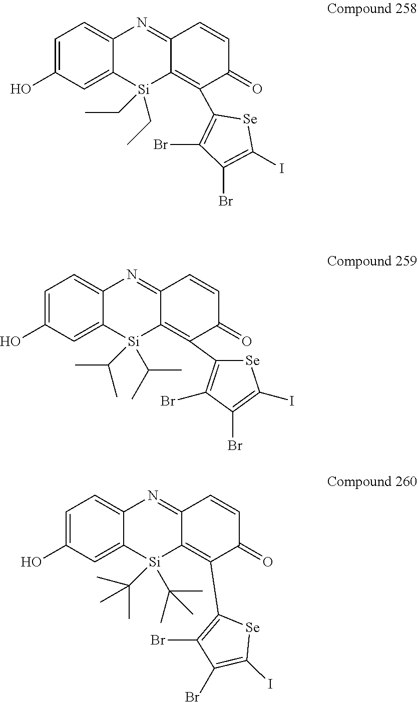 [SMILES: CC[Si]1(CC)C2=C(c3[se]c(I)c(Br)c3Br)C(=O)C=CC2=Nc2ccc(O)cc21 ; CC(C)[Si]1(C(C)C)C2=C(c3[se]c(I)c(Br)c3Br)C(=O)C=CC2=Nc2ccc(O)cc21 ; CC(C)(C)[Si]1(C(C)(C)C)C2=C(c3[se]c(I)c(Br)c3Br)C(=O)C=CC2=Nc2ccc(O)cc21]

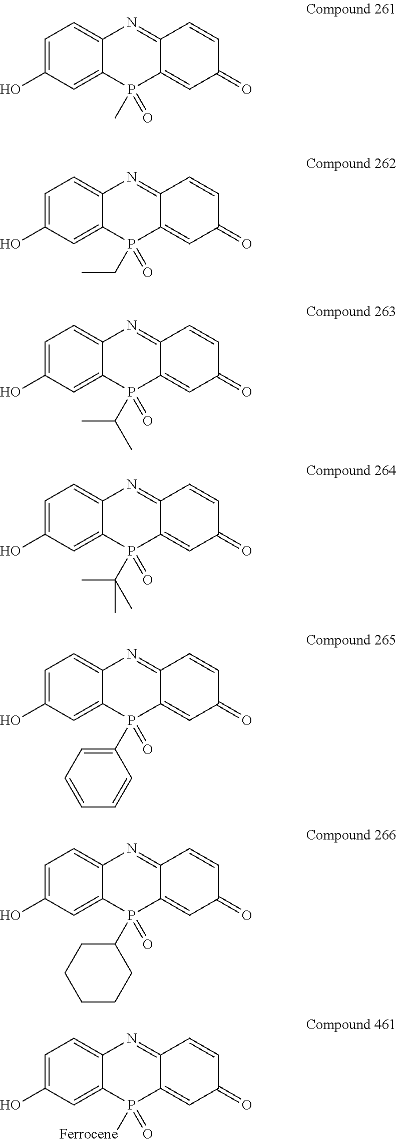

### Synthesized Compounds for Use in Imaging of Cancer Cells:

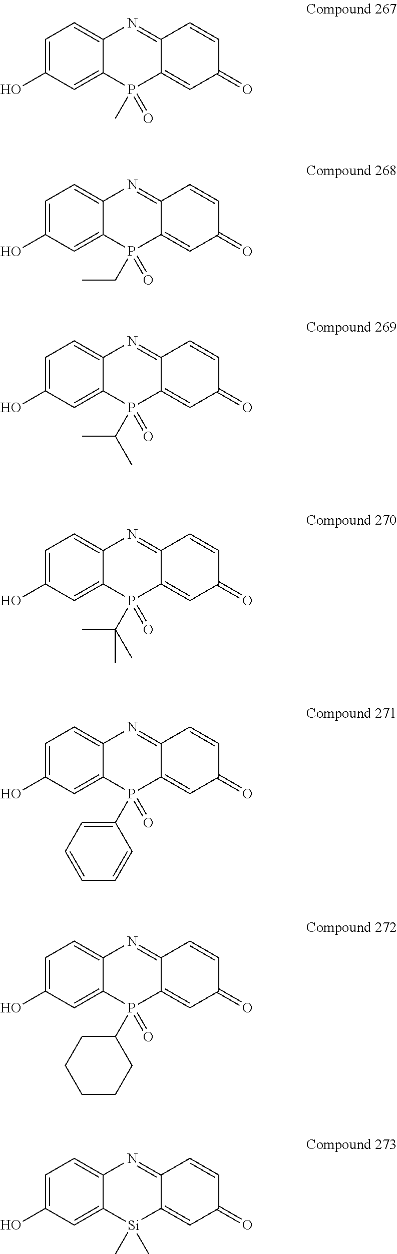

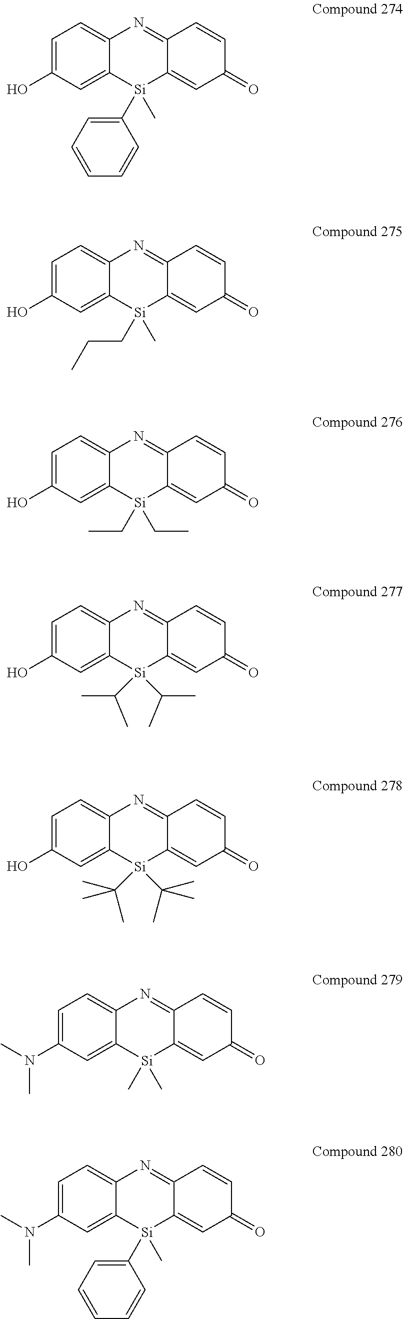

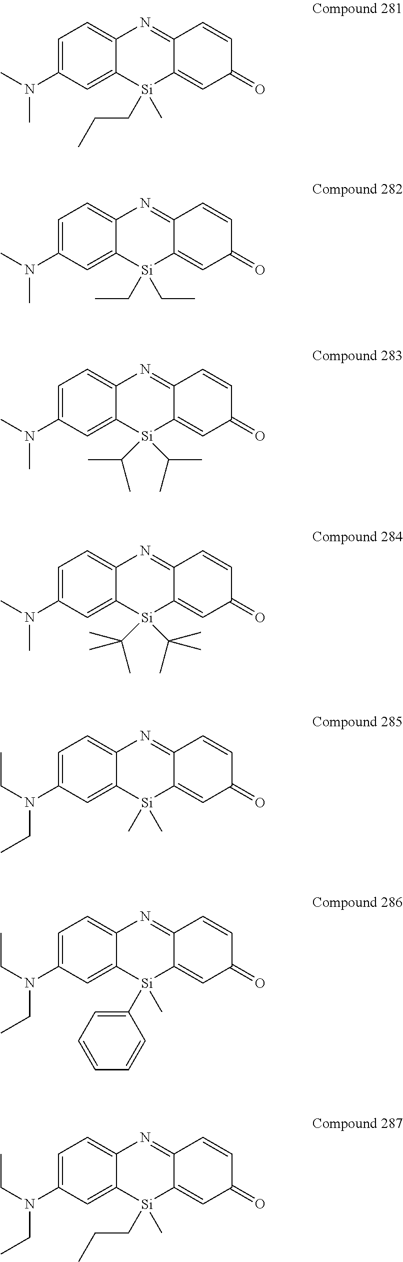

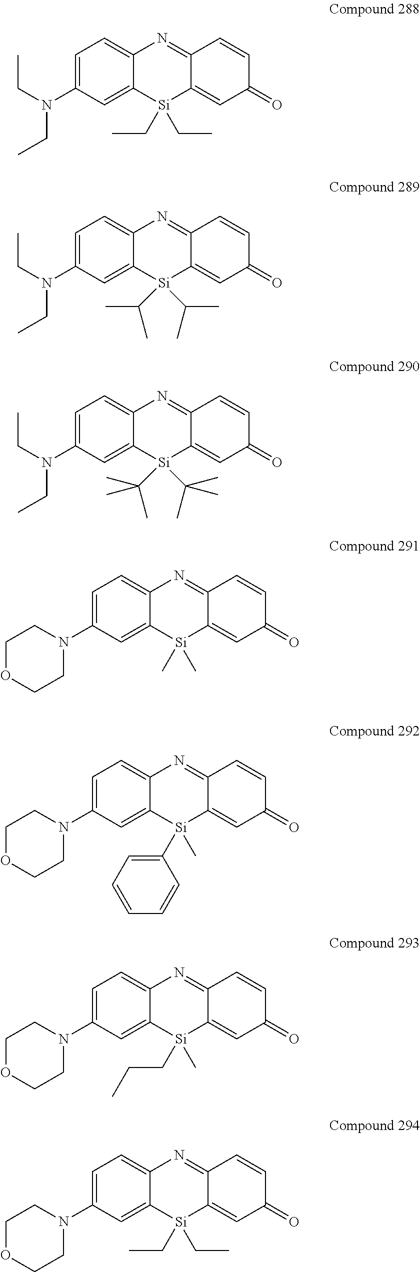

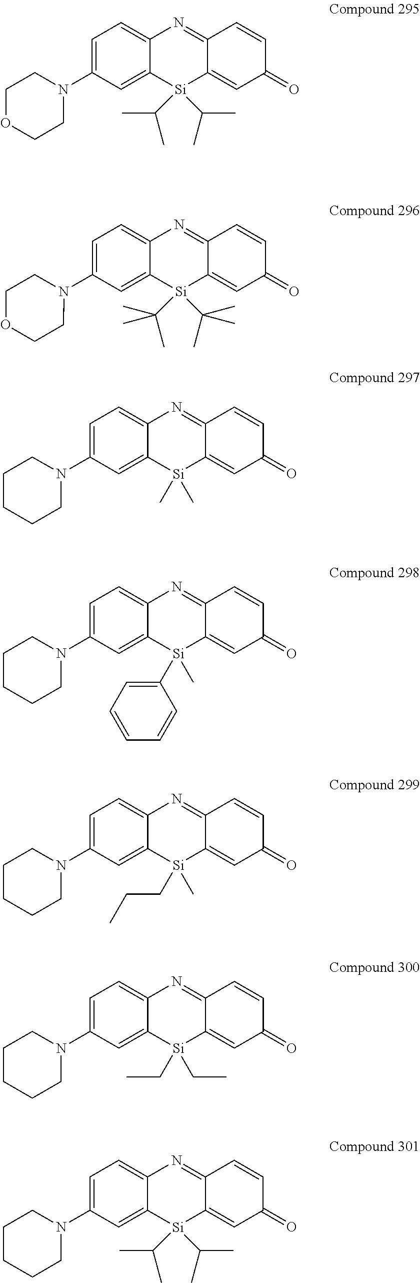

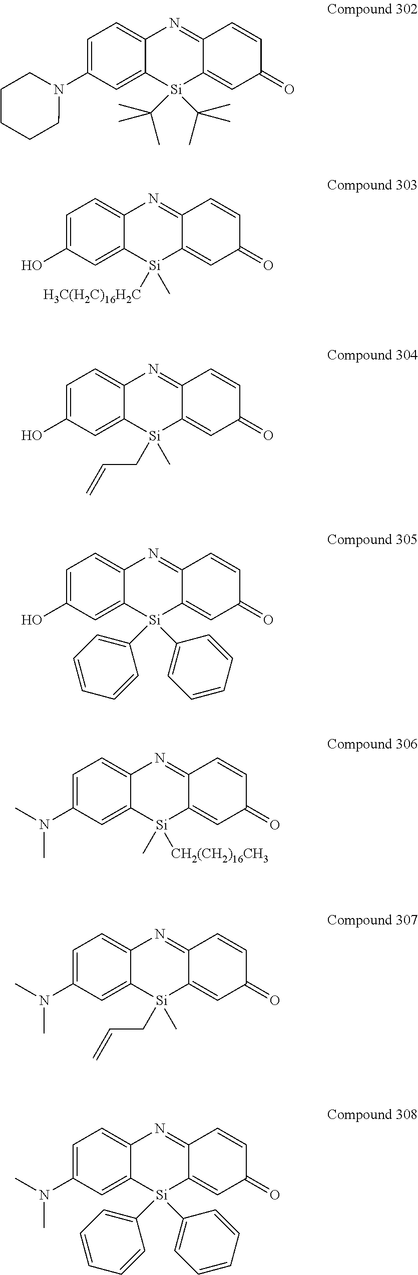

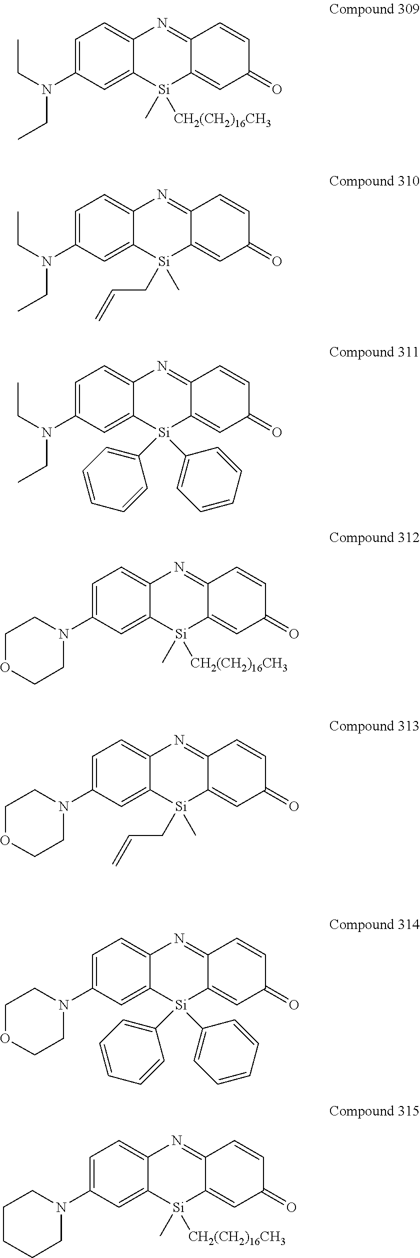

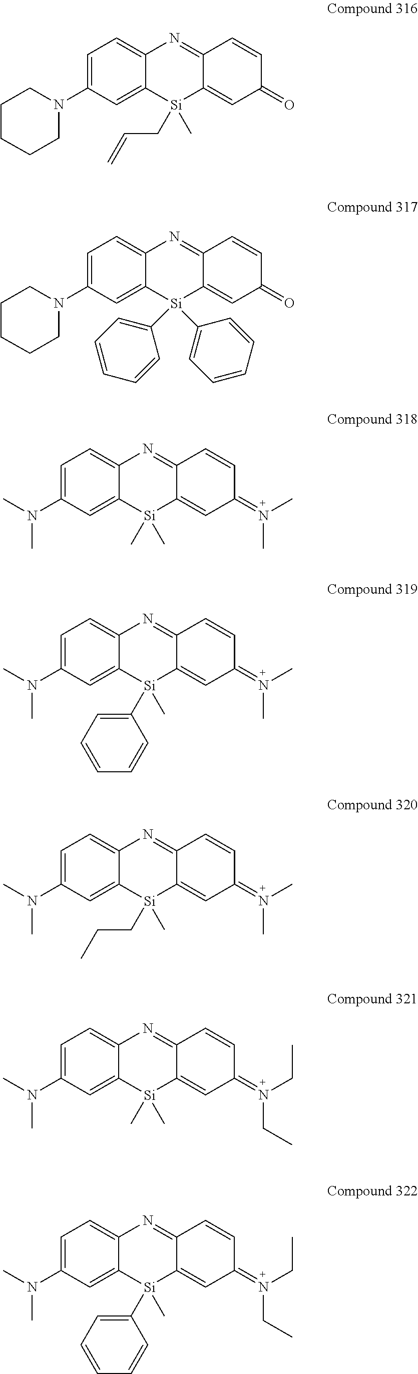

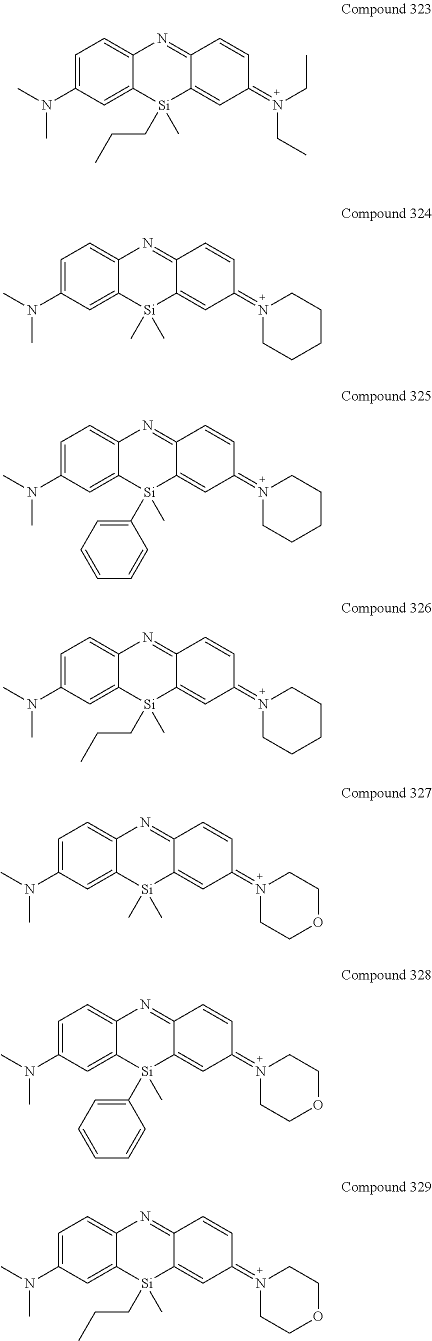

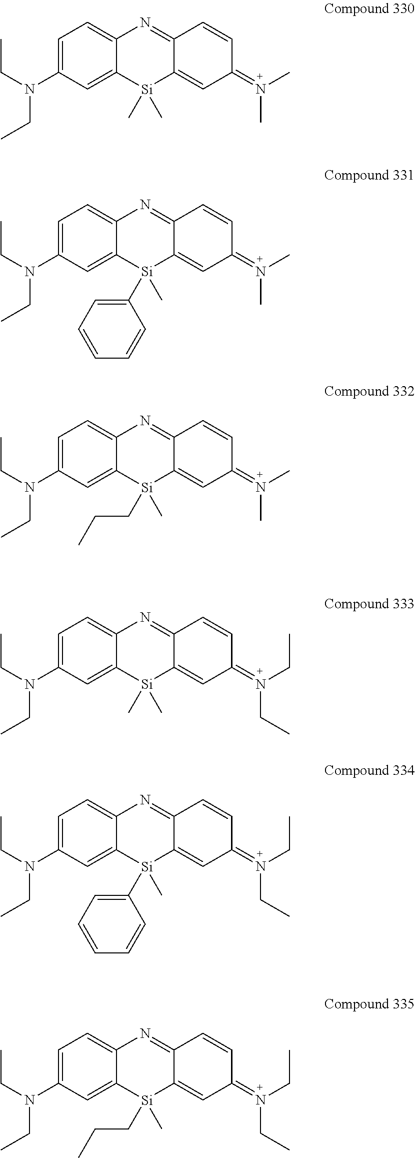

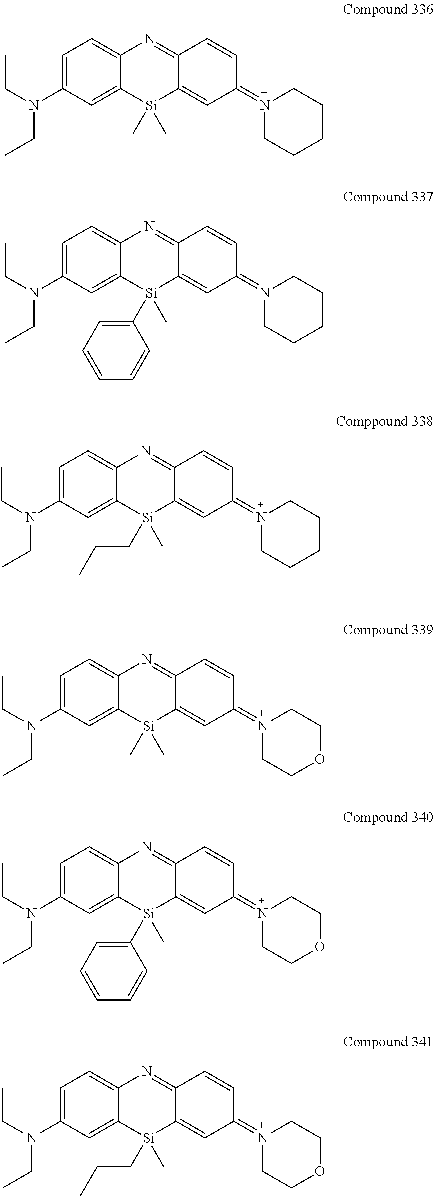

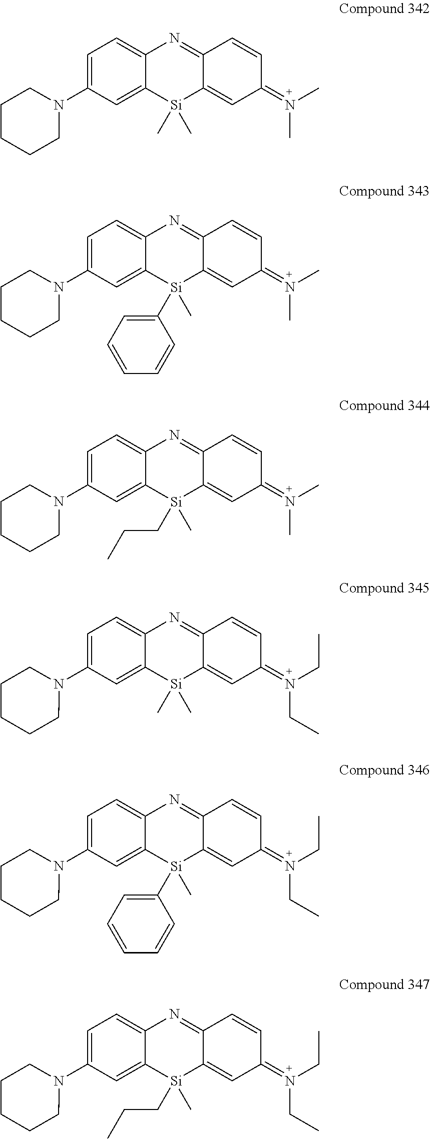

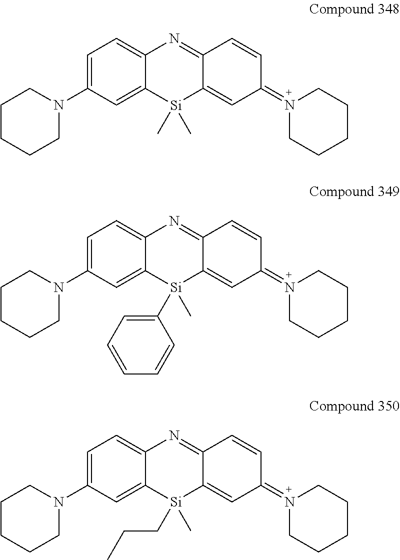

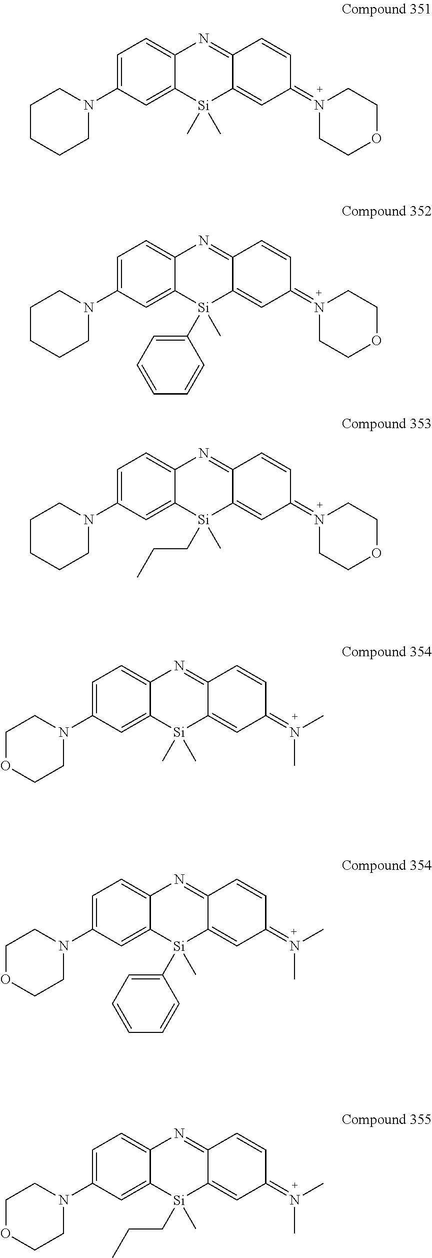

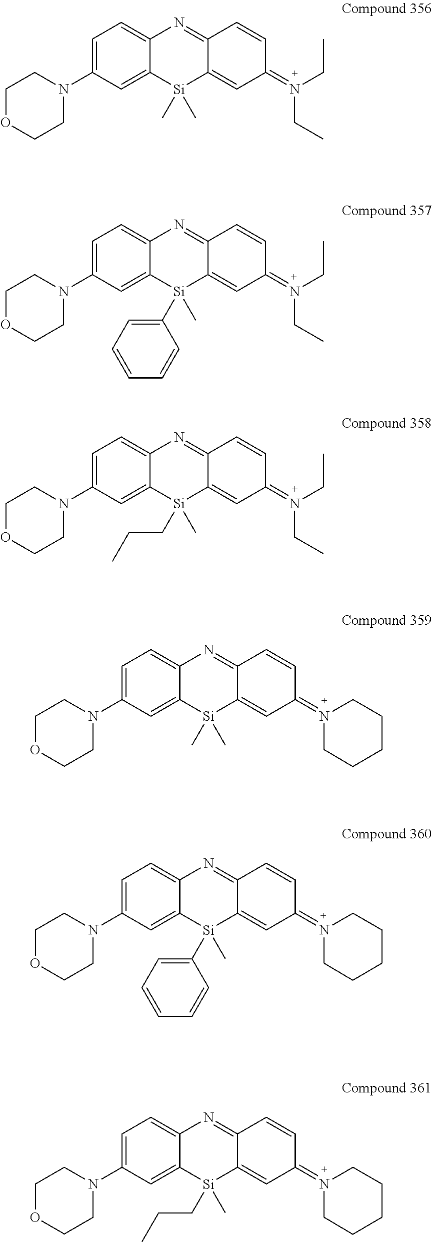

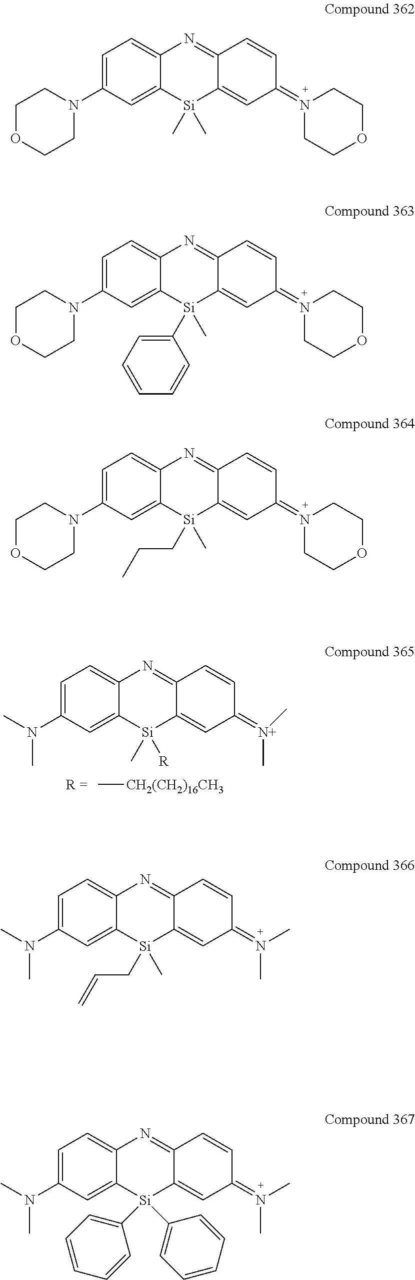

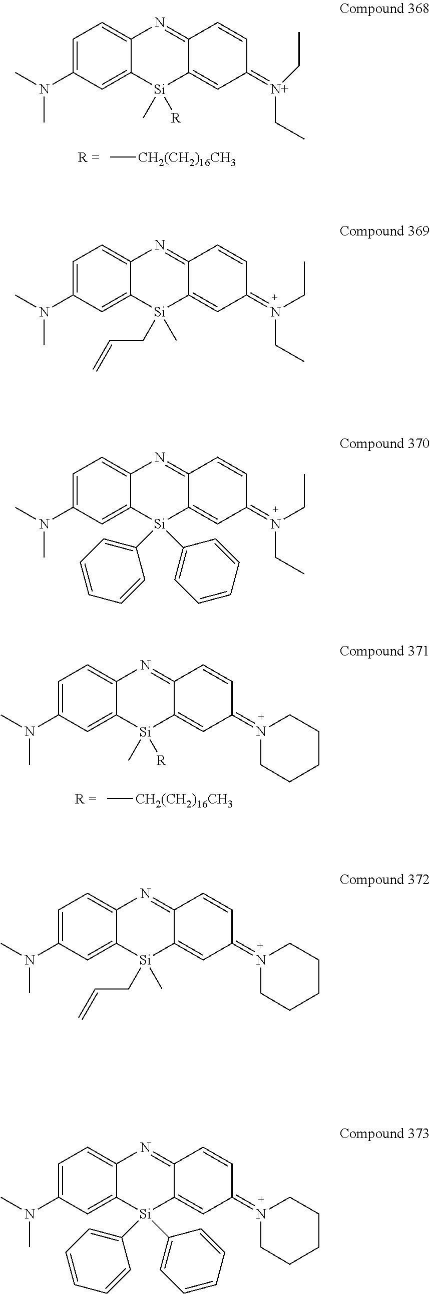

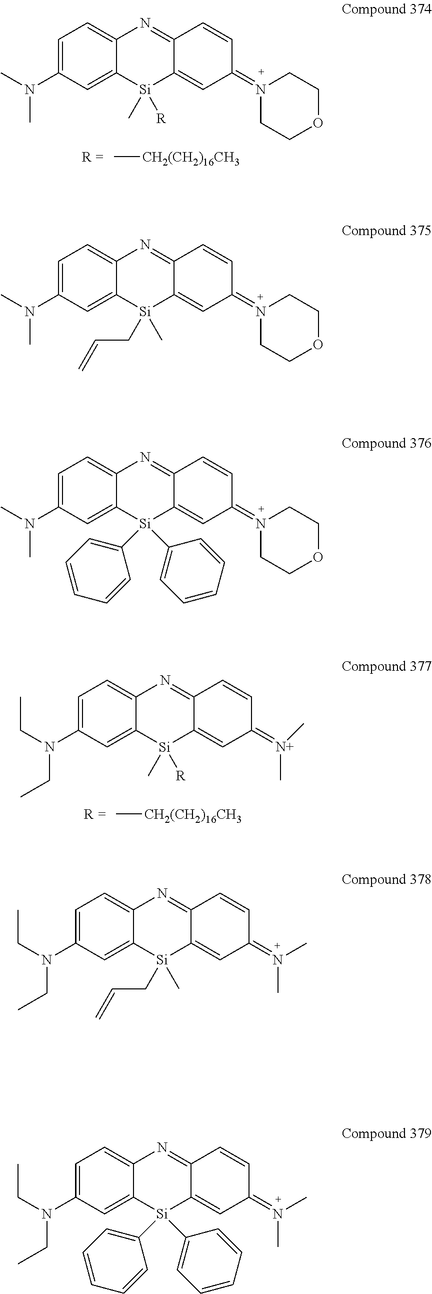

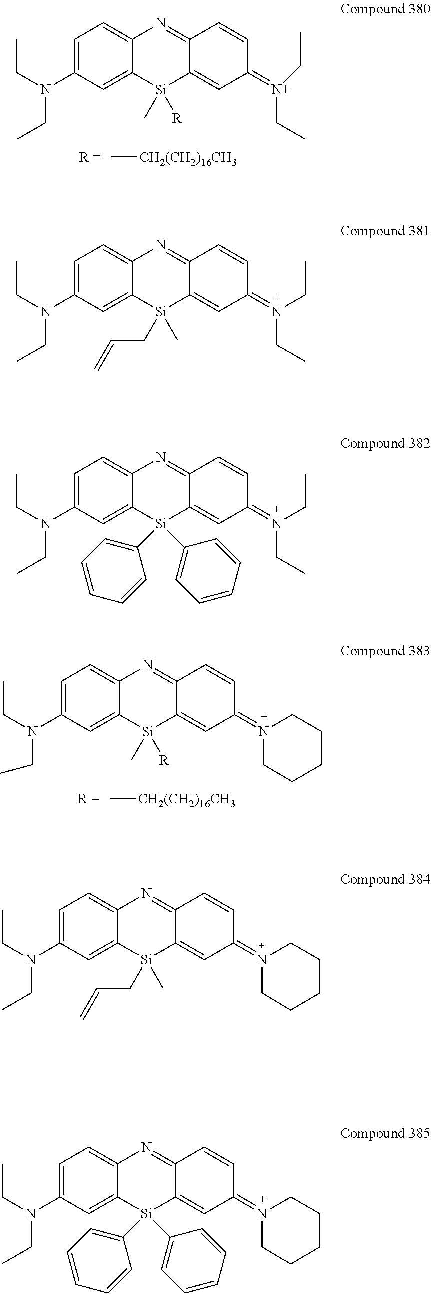

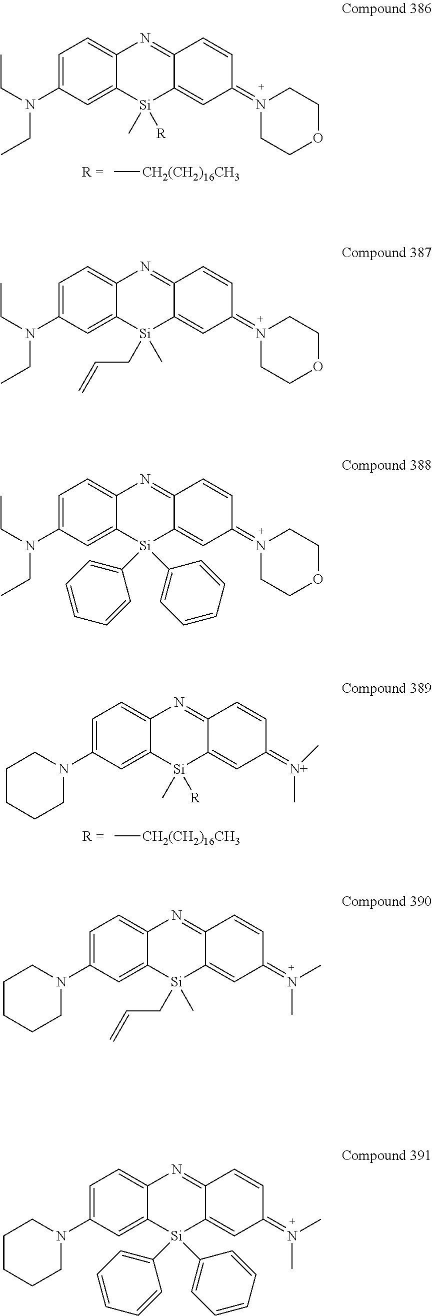

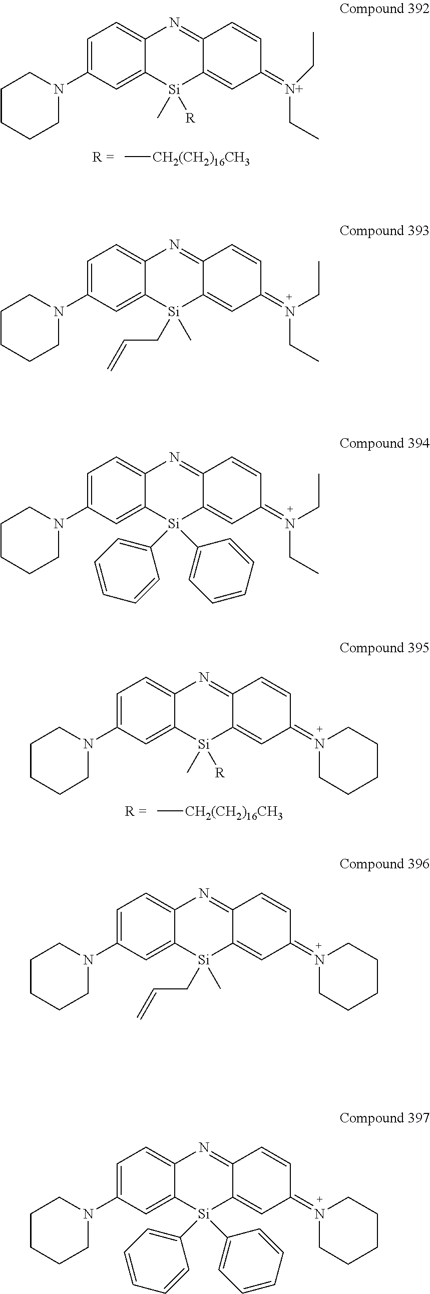

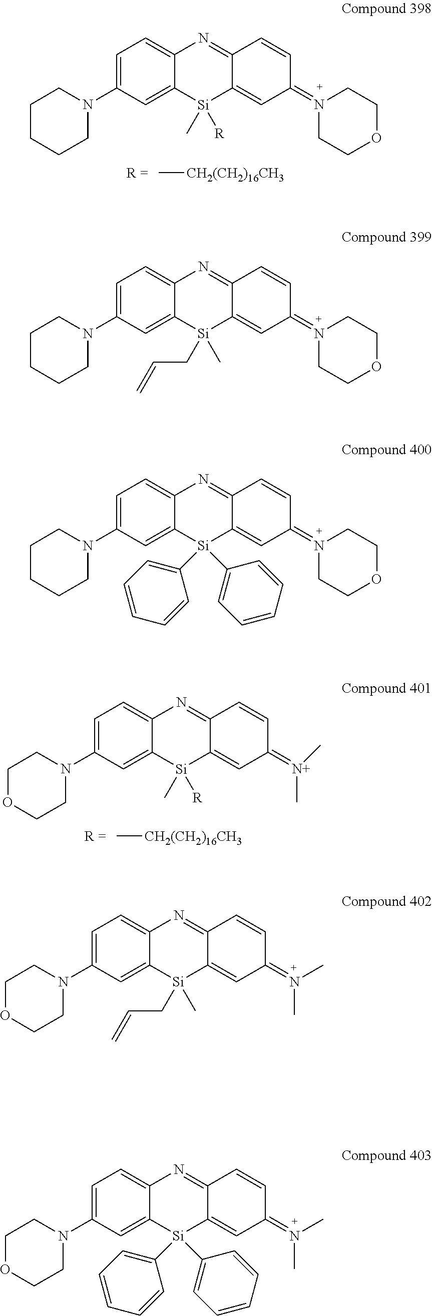

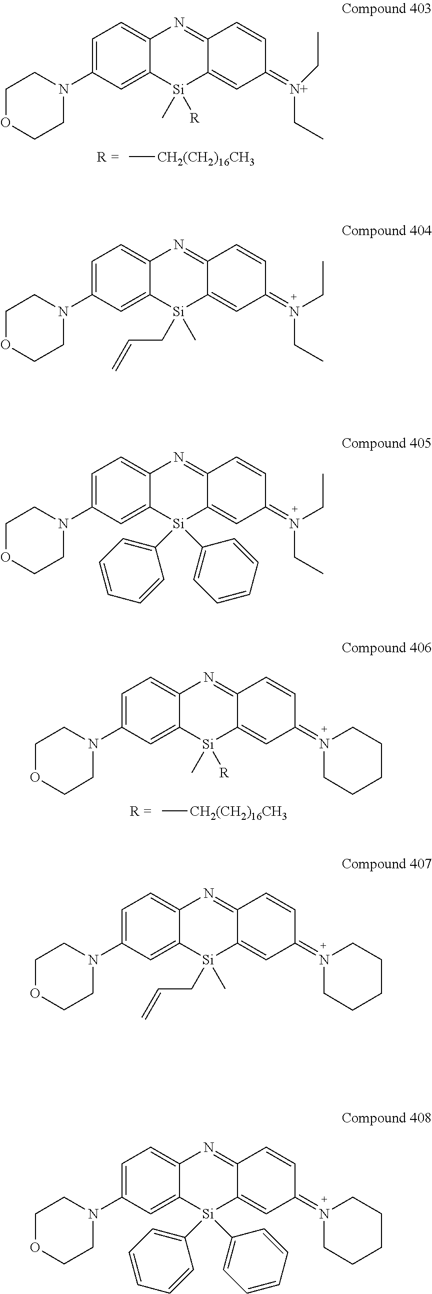

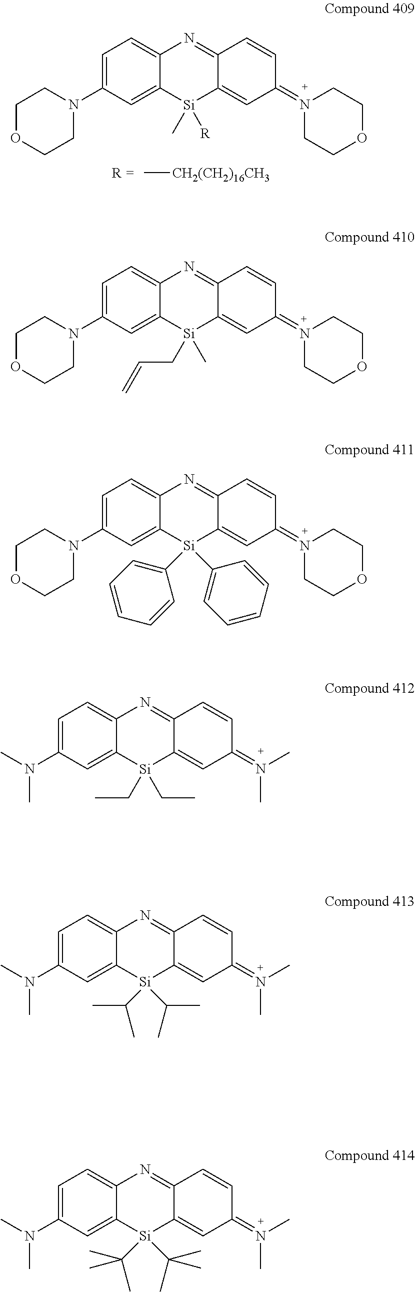

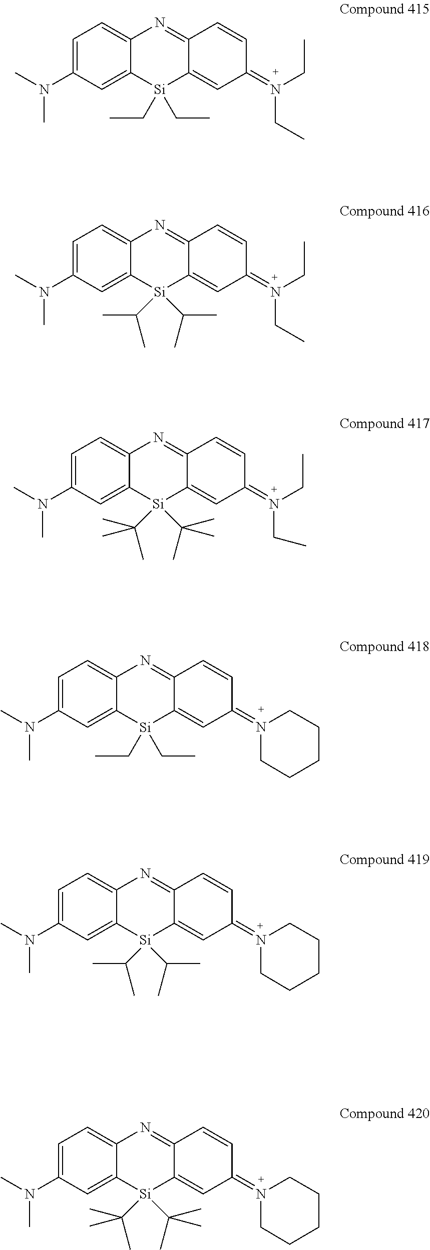

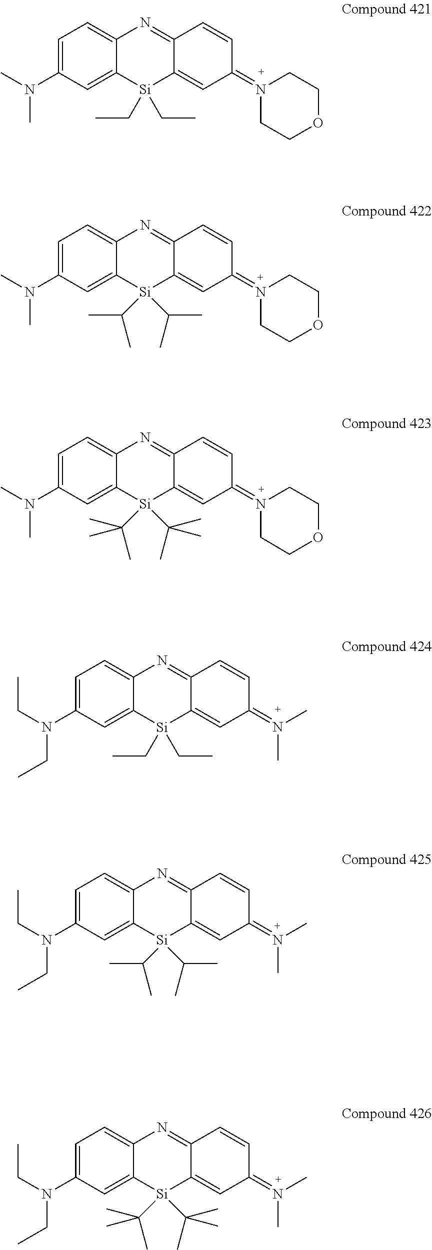

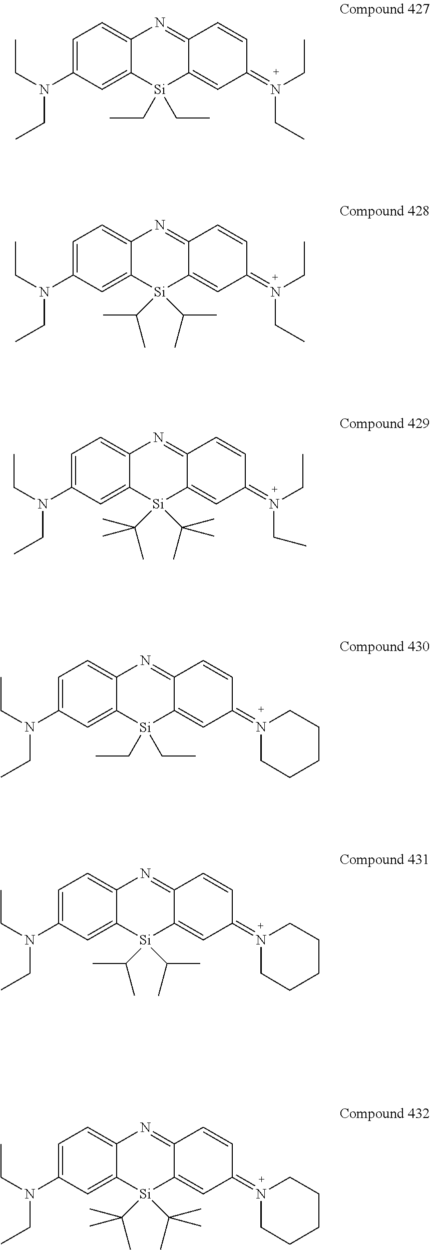

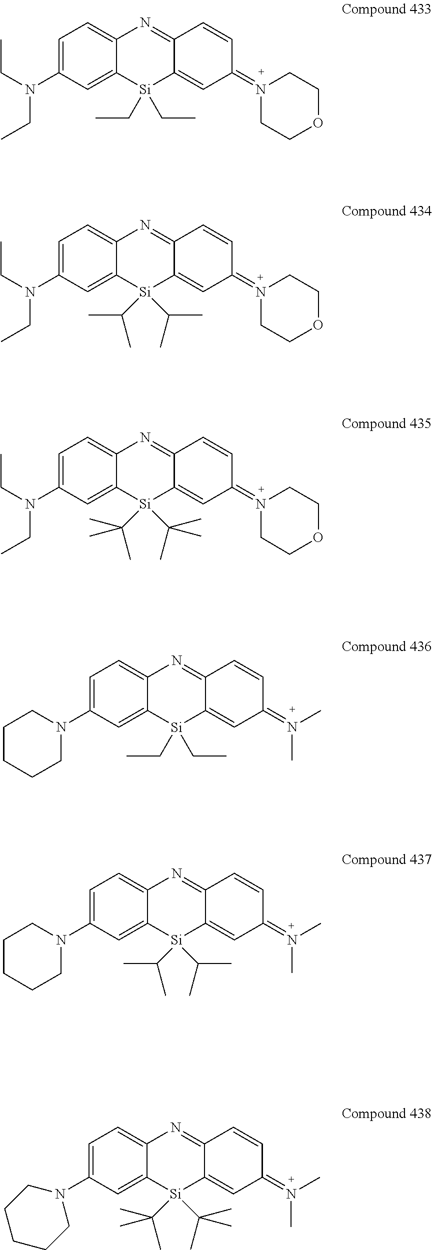

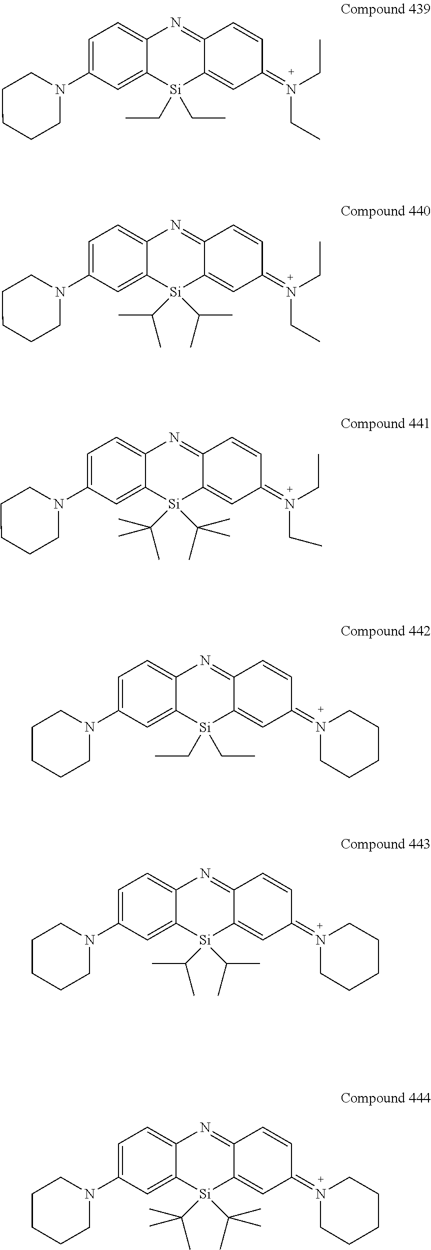

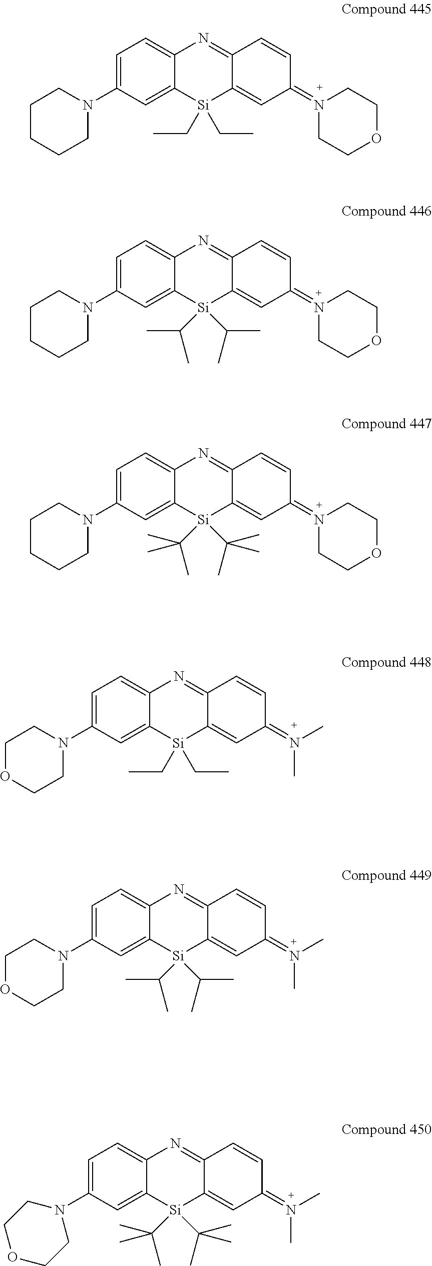

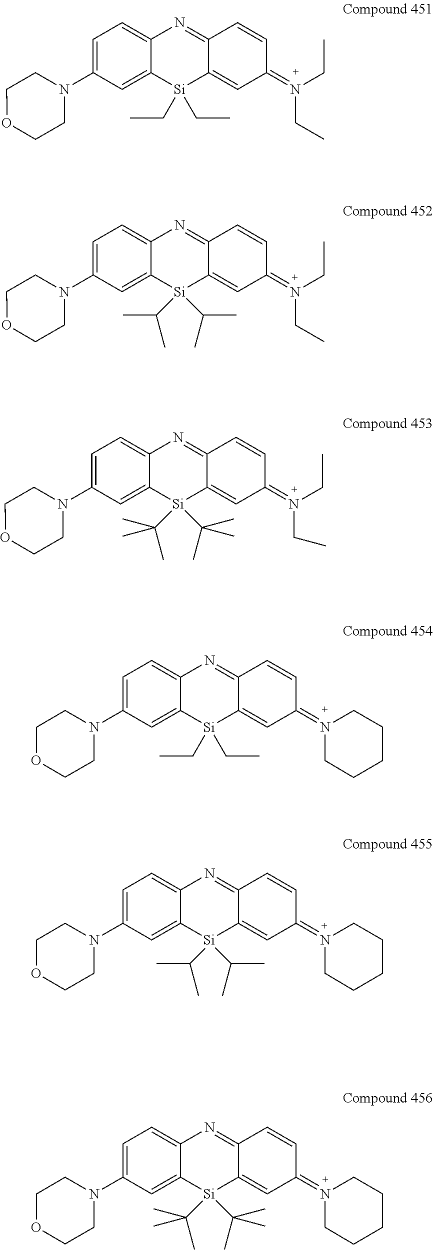

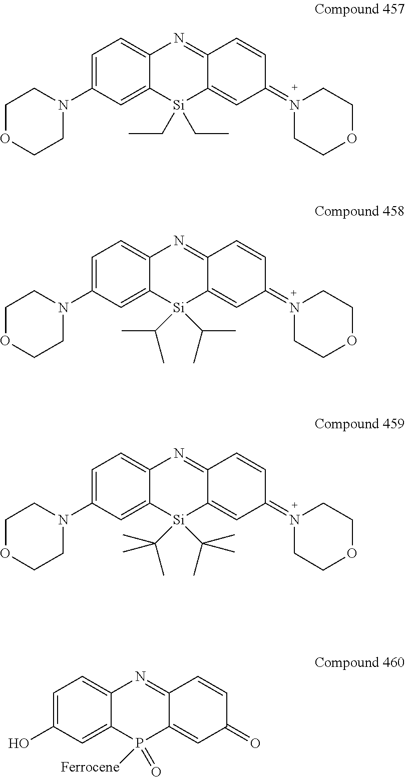

1. A NIR absorbing photosensitizer, comprising formula C or D or E or F or G or H: 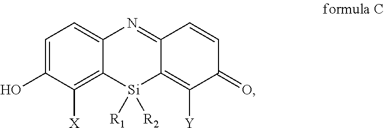
wherein; 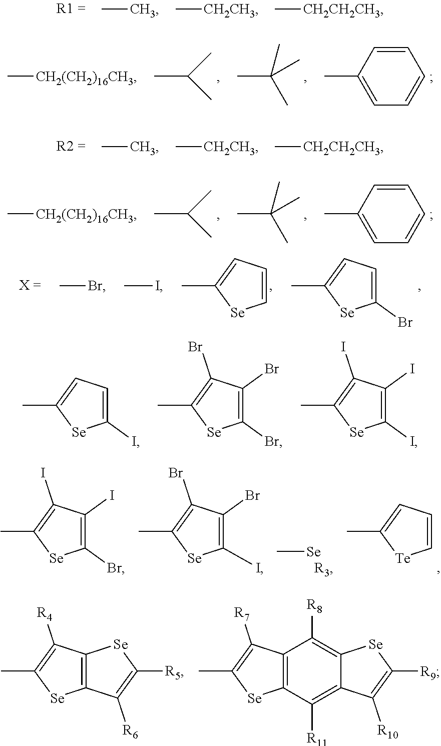
wherein
R₃=alkyl and aryl derivatives;
R₄=—H, —Br, —I, alkyl and aryl derivatives;
R₅=—H, —Br, —I, alkyl and aryl derivatives;
R₆=—H, —Br, —I, alkyl and aryl derivatives;
R₇=—H, —Br, —I, any other possible alkyl and aryl derivatives;
R₈=—H, —Br, —I, any other possible alkyl and aryl derivatives;
R₉=—H, —Br, —I, any other possible alkyl and aryl derivatives;
R₁₀=—H, —Br, —I, any other possible alkyl and aryl derivatives; and
R₁₁=—H, —Br, —I, any other possible alkyl and aryl derivatives; 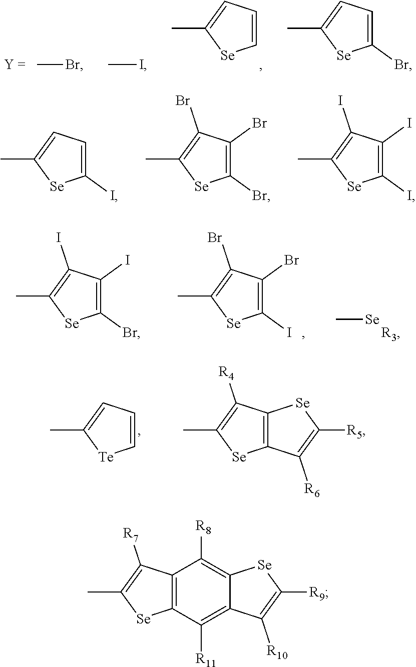
wherein
R₃=alkyl and aryl derivatives;
R₄=—H, —Br, —I, alkyl and aryl derivatives;
R₅=—H, —Br, —I, alkyl and aryl derivatives;
R₆=—H, —Br, —I, alkyl and aryl derivatives;
R₇=—H, —Br, —I, any other possible alkyl and aryl derivatives;
R₈=—H, —Br, —I, any other possible alkyl and aryl derivatives;
R₉=—H, —Br, —I, any other possible alkyl and aryl derivatives;
R₁₀=—H, —Br, —I, any other possible alkyl and aryl derivatives; and
R₁₁=—H, —Br, —I, any other possible alkyl and aryl derivatives; 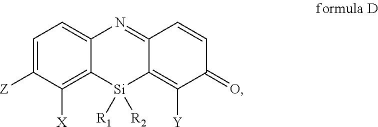
wherein; 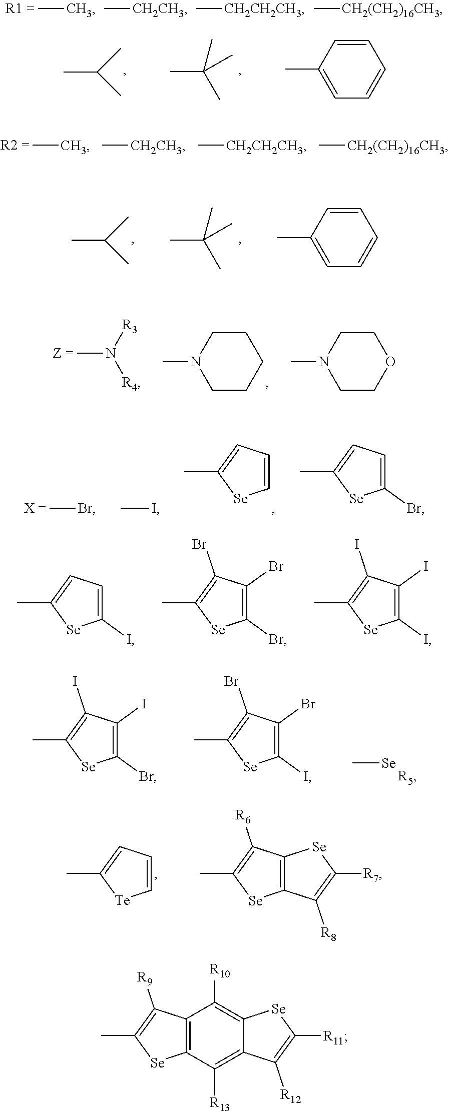
wherein
R₃=alkyl and aryl derivatives;
R₄=alkyl and aryl derivatives;
R₅=alkyl and aryl derivatives;
R₆=—H, —Br, —I, alkyl and aryl derivatives;
R₇=—H, —Br, —I, any other possible alkyl and aryl derivatives;
R₈=—H, —Br, —I, any other possible alkyl and aryl derivatives;
R₉=—H, —Br, —I, any other possible alkyl and aryl derivatives;
R₁₀=—H, —Br, —I, any other possible alkyl and aryl derivatives;
R₁₁=—H, —Br, —I, any other possible alkyl and aryl derivatives;
R₁₂=—H, —Br, —I, any other possible alkyl and aryl derivatives; and
R₁₃=—H, —Br, —I, any other possible alkyl and aryl derivatives; 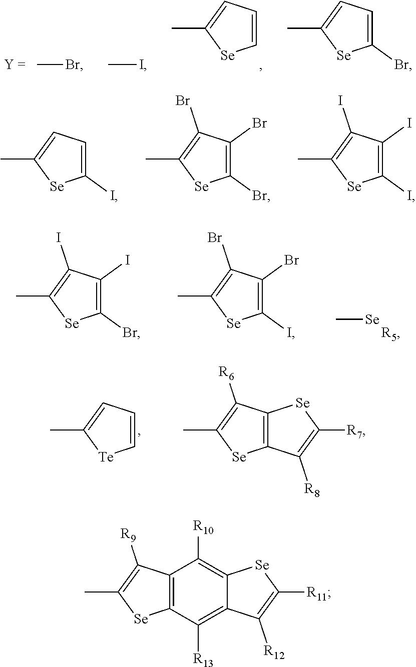
wherein
R₅=alkyl and aryl derivatives;
R₆=—H, —Br, —I, alkyl and aryl derivatives;
R₇=—H, —Br, —I, any other possible alkyl and aryl derivatives;
R₈=—H, —Br, —I, any other possible alkyl and aryl derivatives;
R₉=—H, —Br, —I, any other possible alkyl and aryl derivatives;
R₁₀=—H, —Br, —I, any other possible alkyl and aryl derivatives;
R₁₁=—H, —Br, —I, any other possible alkyl and aryl derivatives;
R₁₂=—H, —Br, —I, any other possible alkyl and aryl derivatives; and
R₁₃=—H, —Br, —I, any other possible alkyl and aryl derivatives; 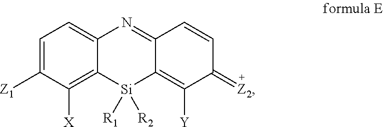
wherein; 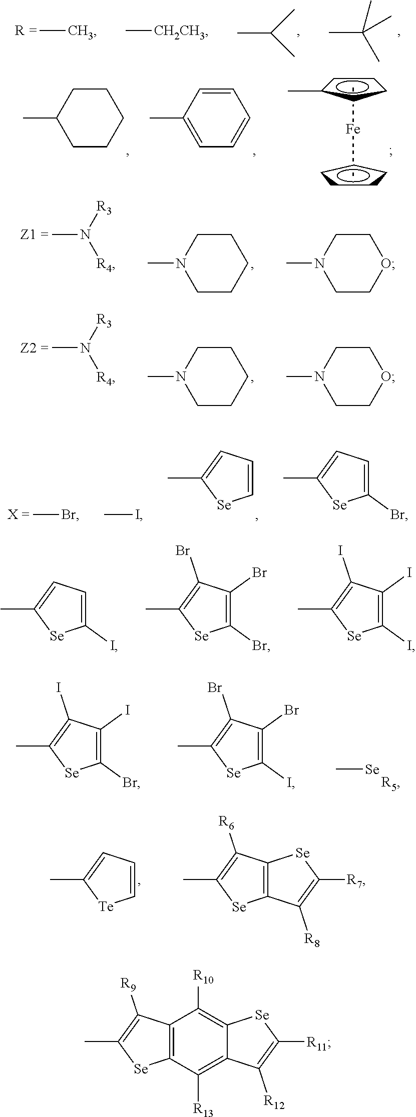
wherein
R₃=alkyl and aryl derivatives;
R₄=alkyl and aryl derivatives;
R₅=alkyl and aryl derivatives;
R₆=—H, —Br, —I, alkyl and aryl derivatives;
R₇=—H, —Br, —I, any other possible alkyl and aryl derivatives;
R₈=—H, —Br, —I, any other possible alkyl and aryl derivatives;
R₉=—H, —Br, —I, any other possible alkyl and aryl derivatives;
R₁₀=—H, —Br, —I, any other possible alkyl and aryl derivatives;
R₁₁=—H, —Br, —I, any other possible alkyl and aryl derivatives;
R₁₂=—H, —Br, —I, any other possible alkyl and aryl derivatives; and
R₁₃=—H, —Br, —I, any other possible alkyl and aryl derivatives; 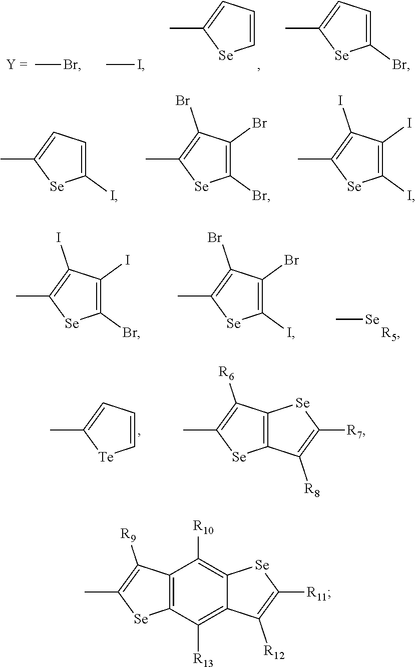
wherein
R₅=alkyl and aryl derivatives;
R₆=—H, —Br, —I, alkyl and aryl derivatives;
R₇=—H, —Br, —I, any other possible alkyl and aryl derivatives;
R₈=—H, —Br, —I, any other possible alkyl and aryl derivatives;
R₉=—H, —Br, —I, any other possible alkyl and aryl derivatives;
R₁₀=—H, —Br, —I, any other possible alkyl and aryl derivatives;
R₁₁=—H, —Br, —I, any other possible alkyl and aryl derivatives;
R₁₂=—H, —Br, —I, any other possible alkyl and aryl derivatives; and
R₁₃=—H, —Br, —I, any other possible alkyl and aryl derivatives; 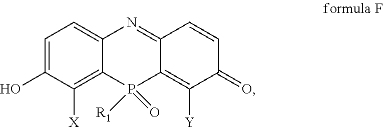
wherein; 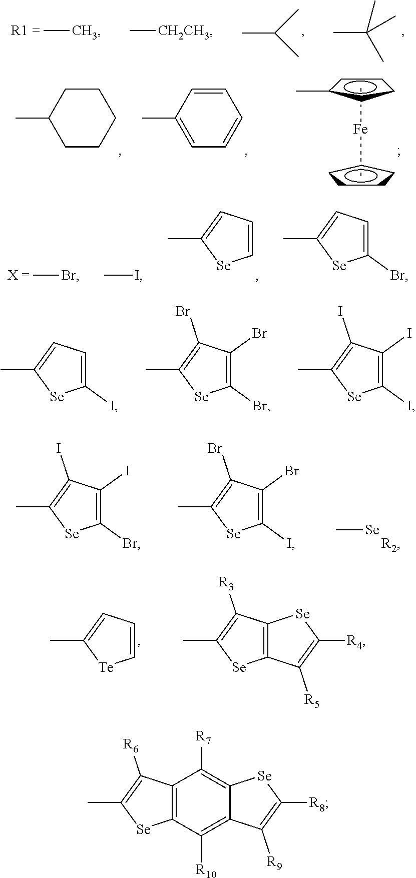
wherein
R₂=alkyl and aryl derivatives;
R₃=—H, —Br, —I, alkyl and aryl derivatives;
R₄=—H, —Br, —I, any other possible alkyl and aryl derivatives;
R₅=—H, —Br, —I, any other possible alkyl and aryl derivatives;
R₆=—H, —Br, —I, any other possible alkyl and aryl derivatives;
R₇=—H, —Br, —I, any other possible alkyl and aryl derivatives;
R₈=—H, —Br, —I, any other possible alkyl and aryl derivatives;
R₉=—H, —Br, —I, any other possible alkyl and aryl derivatives; and
R₁₀=—H, —Br, —I, any other possible alkyl and aryl derivatives; 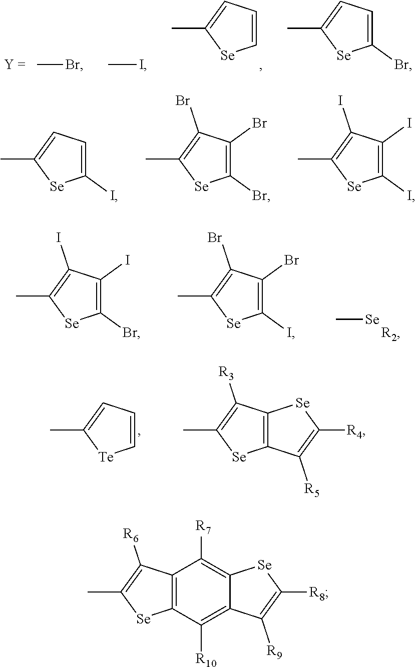
wherein
R₂=alkyl and aryl derivatives;
R₃=—H, —Br, —I, alkyl and aryl derivatives;
R₄=—H, —Br, —I, any other possible alkyl and aryl derivatives;
R₅=—H, —Br, —I, any other possible alkyl and aryl derivatives;
R₆=—H, —Br, —I, any other possible alkyl and aryl derivatives;
R₇=—H, —Br, —I, any other possible alkyl and aryl derivatives;
R₈=—H, —Br, —I, any other possible alkyl and aryl derivatives;
R₉=—H, —Br, —I, any other possible alkyl and aryl derivatives; and
R₁₀=—H, —Br, —I, any other possible alkyl and aryl derivatives; 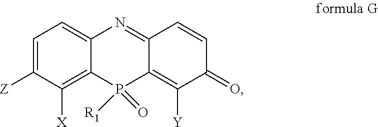
wherein: 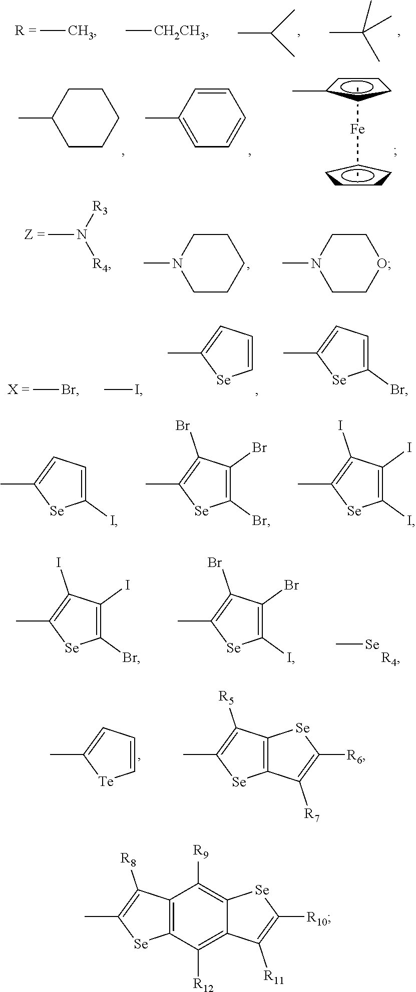
wherein
R₃=alkyl and aryl derivatives;
R₄=alkyl and aryl derivatives;
R₅=—H, —Br, —I, alkyl and aryl derivatives;
R₆=—H, —Br, —I, any other possible alkyl and aryl derivatives;
R₇=—H, —Br, —I, any other possible alkyl and aryl derivatives;
R₈=—H, —Br, —I, any other possible alkyl and aryl derivatives;
R₉=—H, —Br, —I, any other possible alkyl and aryl derivatives;
R₁₀=—H, —Br, —I, any other possible alkyl and aryl derivatives;
R₁₁=—H, —Br, —I, any other possible alkyl and aryl derivatives; and
R₁₂=—H, —Br, —I, any other possible alkyl and aryl derivatives; 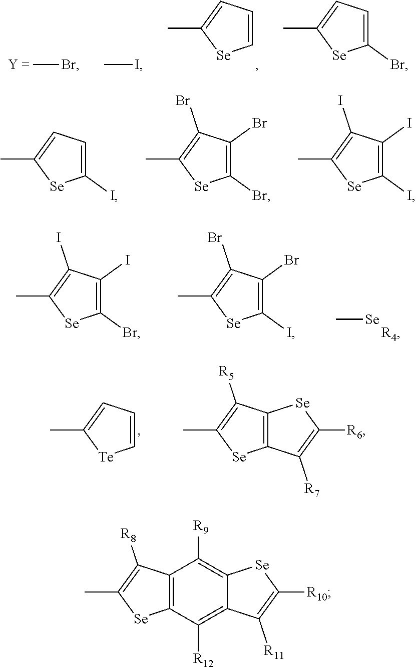
wherein
R₄=alkyl and aryl derivatives;
R₅=—H, —Br, —I, alkyl and aryl derivatives;
R₆=—H, —Br, —I, alkyl and aryl derivatives;
R₇=—H, —Br, —I, any other possible alkyl and aryl derivatives;
R₈=—H, —Br, —I, any other possible alkyl and aryl derivatives;
R₉=—H, —Br, —I, any other possible alkyl and aryl derivatives;
R₁₀=—H, —Br, —I, any other possible alkyl and aryl derivatives;
R₁₁=—H, —Br, —I, any other possible alkyl and aryl derivatives; and
R₁₂=—H, —Br, —I, any other possible alkyl and aryl derivatives; 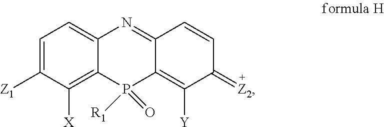
wherein; 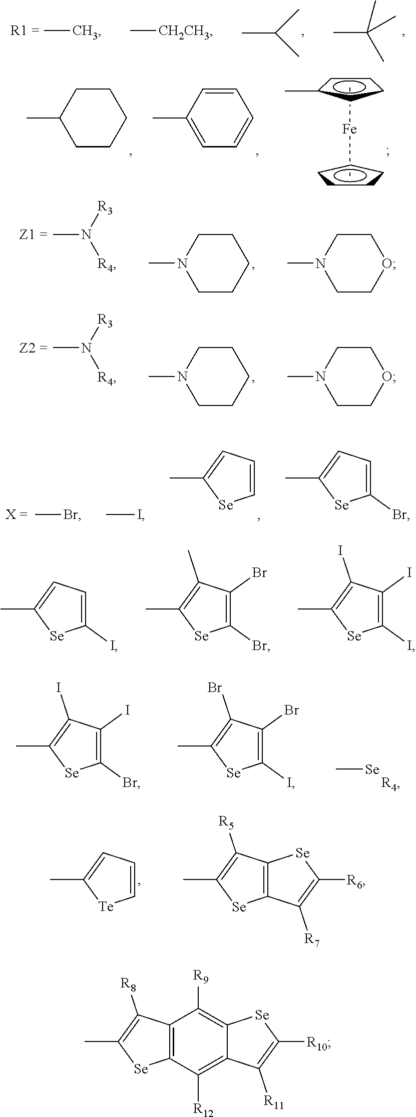
wherein
R₃=alkyl and aryl derivatives;
R₄=alkyl and aryl derivatives;
R₅=—H, —Br, —I, alkyl and aryl derivatives;
R₆=—H, —Br, —I, any other possible alkyl and aryl derivatives;
R₇=—H, —Br, —I, any other possible alkyl and aryl derivatives;
R₉=—H, —Br, —I, any other possible alkyl and aryl derivatives;
R₁₀=—H, —Br, —I, any other possible alkyl and aryl derivatives;
R₁₁=—H, —Br, —I, any other possible alkyl and aryl derivatives; and
R₁₂=—H, —Br, —I, any other possible alkyl and aryl derivatives; 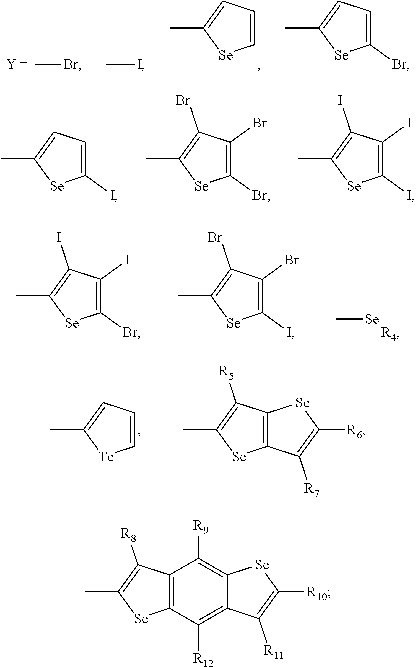
wherein
R₄=alkyl and aryl derivatives;
R₅=—H, —Br, —I, alkyl and aryl derivatives;
R₆=—H, —Br, —I, alkyl and aryl derivatives;
R₇=—H, —Br, —I, any other possible alkyl and aryl derivatives;
R₈=—H, —Br, —I, any other possible alkyl and aryl derivatives;
R₉=—H, —Br, —I, any other possible alkyl and aryl derivatives;
R₁₀=—H, —Br, —I, any other possible alkyl and aryl derivatives;
R₁₁=—H, —Br, —I, any other possible alkyl and aryl derivatives; and
R₁₂=—H, —Br, —I, any other possible alkyl and aryl derivatives.

2. The NIR absorbing photosensitizer according to claim 1, comprising one selected from the group of:

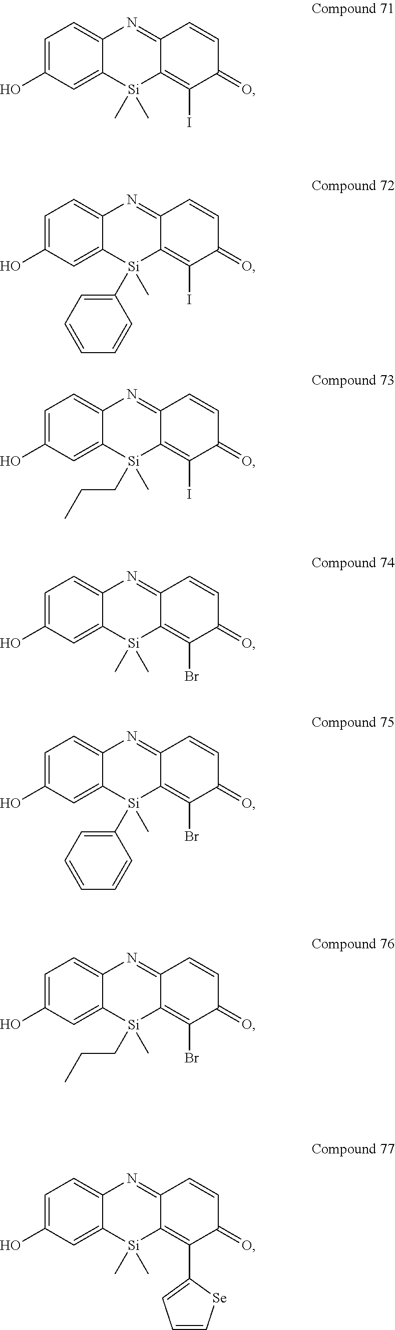

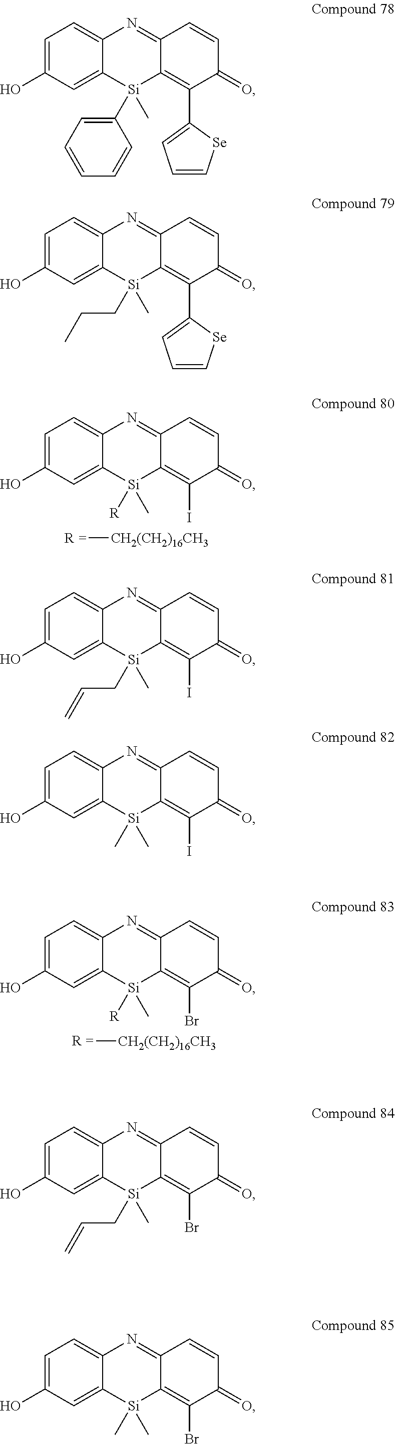

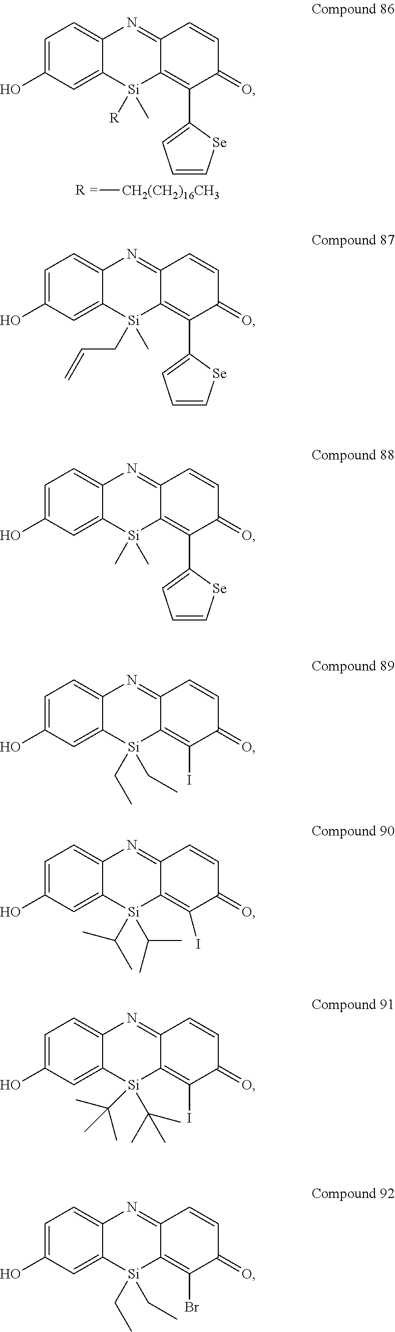

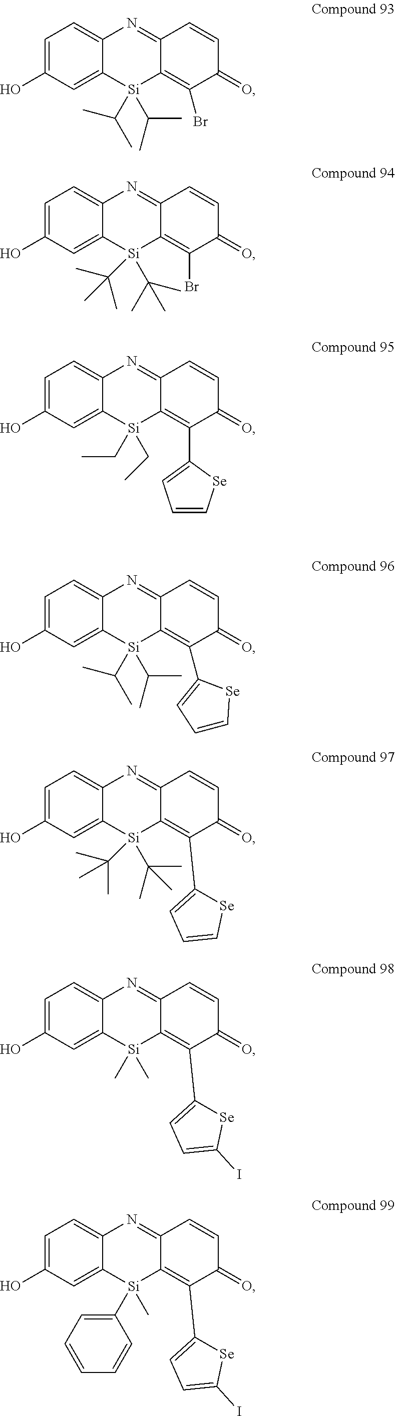

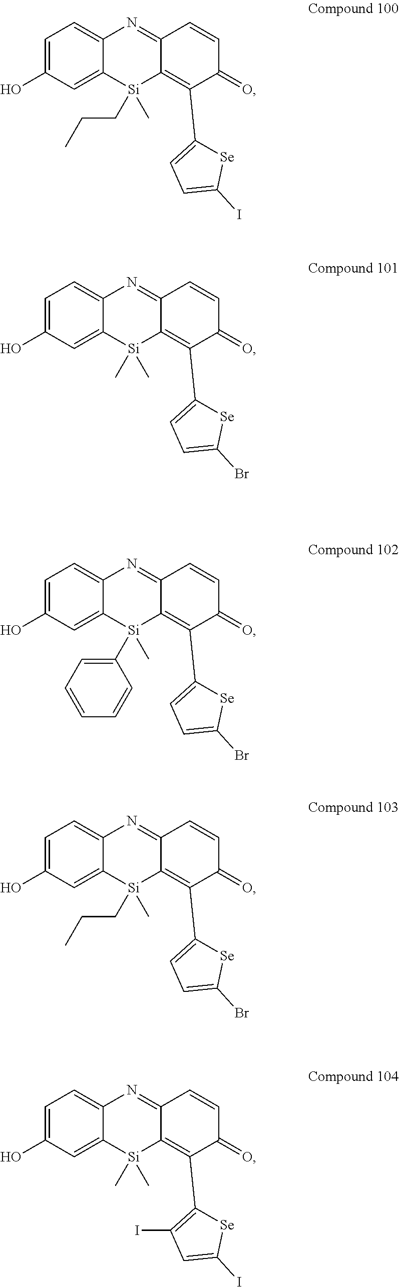 [SMILES: CCC[Si]1(C)C2=C(c3ccc(I)[se]3)C(=O)C=CC2=Nc2ccc(O)cc21 ; C[Si]1(C)C2=C(c3ccc(Br)[se]3)C(=O)C=CC2=Nc2ccc(O)cc21 ; C[Si]1(c2ccccc2)C2=C(c3ccc(Br)[se]3)C(=O)C=CC2=Nc2ccc(O)cc21 ; CCC[Si]1(C)C2=C(c3ccc(Br)[se]3)C(=O)C=CC2=Nc2ccc(O)cc21 ; C[Si]1(C)C2=C(c3[se]c(I)cc3I)C(=O)C=CC2=Nc2ccc(O)cc21]

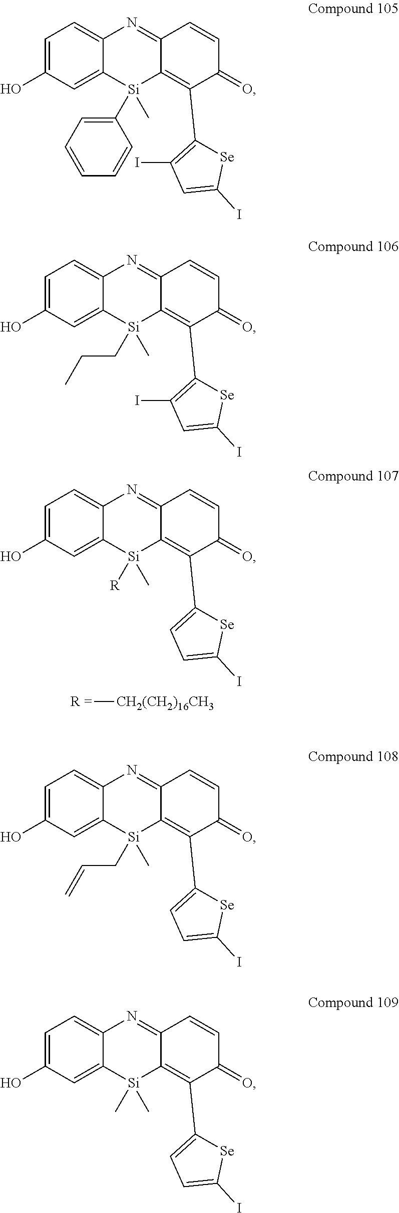

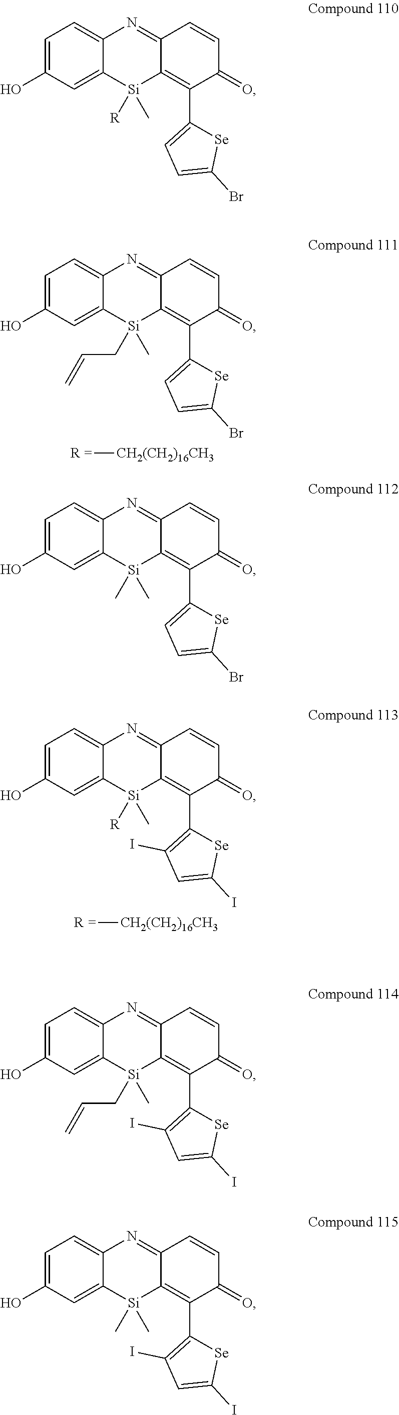

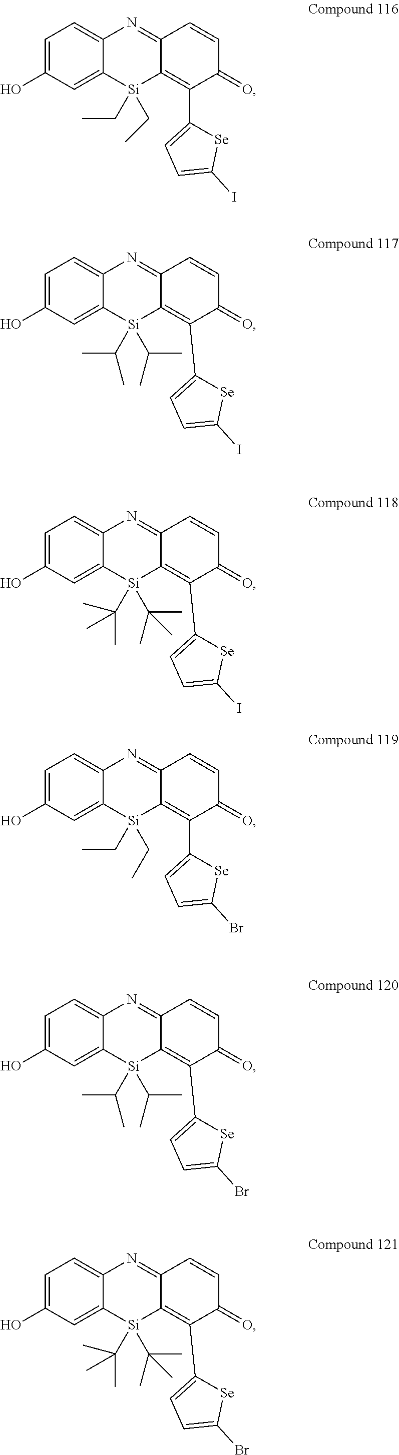 [SMILES: CC[Si]1(CC)C2=C(c3ccc(I)[se]3)C(=O)C=CC2=Nc2ccc(O)cc21 ; CC(C)[Si]1(C(C)C)C2=C(c3ccc(I)[se]3)C(=O)C=CC2=Nc2ccc(O)cc21 ; CC(C)(C)[Si]1(C(C)(C)C)C2=C(c3ccc(I)[se]3)C(=O)C=CC2=Nc2ccc(O)cc21 ; CC[Si]1(CC)C2=C(c3ccc(Br)[se]3)C(=O)C=CC2=Nc2ccc(O)cc21 ; CC(C)[Si]1(C(C)C)C2=C(c3ccc(Br)[se]3)C(=O)C=CC2=Nc2ccc(O)cc21 ; CC(C)(C)[Si]1(C(C)(C)C)C2=C(c3ccc(Br)[se]3)C(=O)C=CC2=Nc2ccc(O)cc21]

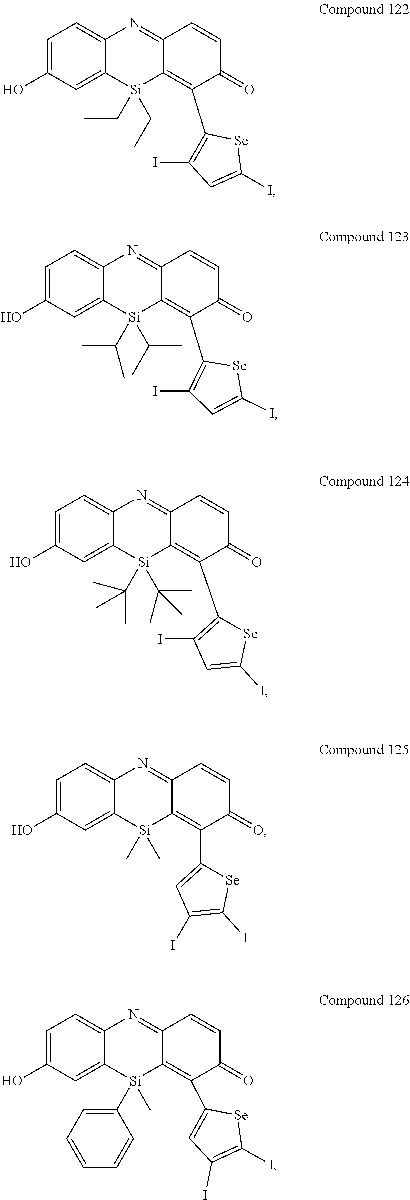 [SMILES: CC[Si]1(CC)C2=C(c3[se]c(I)cc3I)C(=O)C=CC2=Nc2ccc(O)cc21 ; CC(C)[Si]1(C(C)C)C2=C(c3[se]c(I)cc3I)C(=O)C=CC2=Nc2ccc(O)cc21 ; CC(C)(C)[Si]1(C(C)(C)C)C2=C(c3[se]c(I)cc3I)C(=O)C=CC2=Nc2ccc(O)cc21 ; C[Si]1(C)C2=C(c3cc(I)c(I)[se]3)C(=O)C=CC2=Nc2ccc(O)cc21 ; C[Si]1(c2ccccc2)C2=C(c3cc(I)c(I)[se]3)C(=O)C=CC2=Nc2ccc(O)cc21]

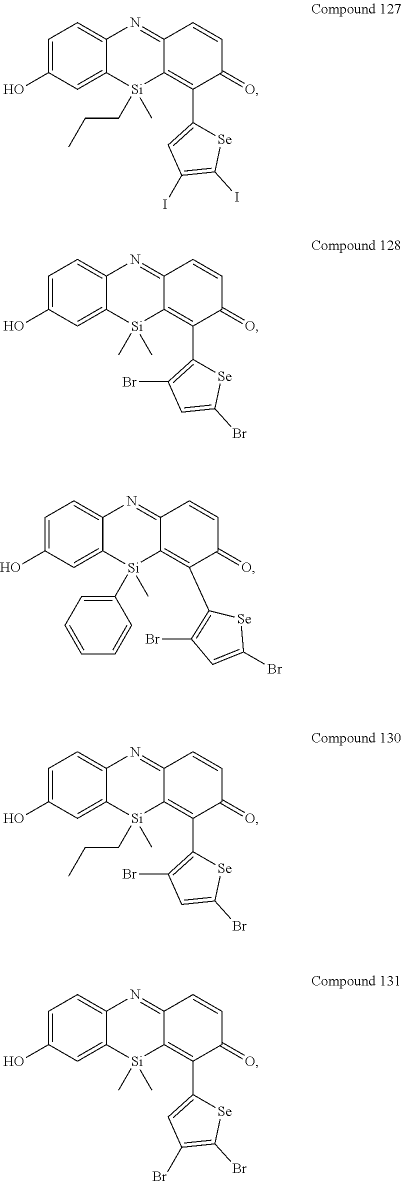 [SMILES: CCC[Si]1(C)C2=C(c3cc(I)c(I)[se]3)C(=O)C=CC2=Nc2ccc(O)cc21 ; C[Si]1(C)C2=C(c3[se]c(Br)cc3Br)C(=O)C=CC2=Nc2ccc(O)cc21 ; C[Si]1(c2ccccc2)C2=C(c3[se]c(Br)cc3Br)C(=O)C=CC2=Nc2ccc(O)cc21 ; CCC[Si]1(C)C2=C(c3[se]c(Br)cc3Br)C(=O)C=CC2=Nc2ccc(O)cc21 ; C[Si]1(C)C2=C(c3cc(Br)c(Br)[se]3)C(=O)C=CC2=Nc2ccc(O)cc21]

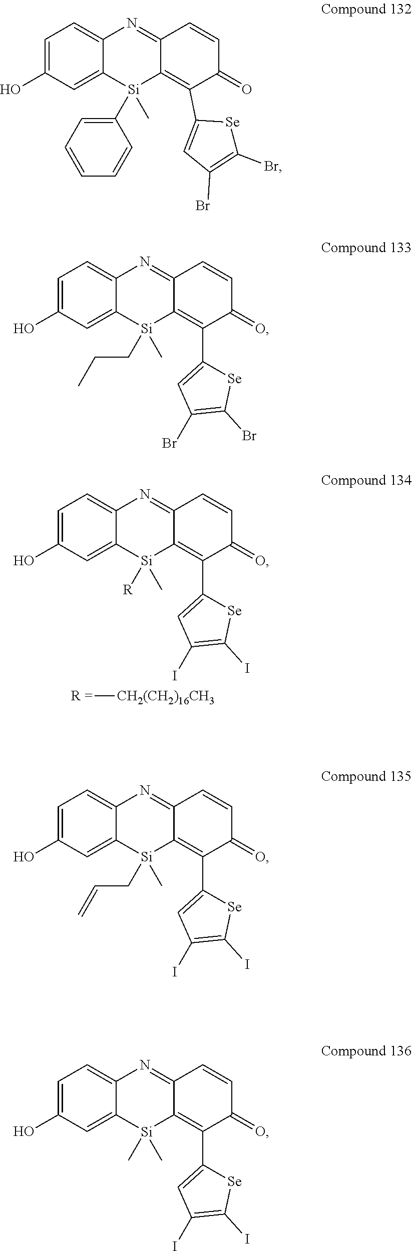

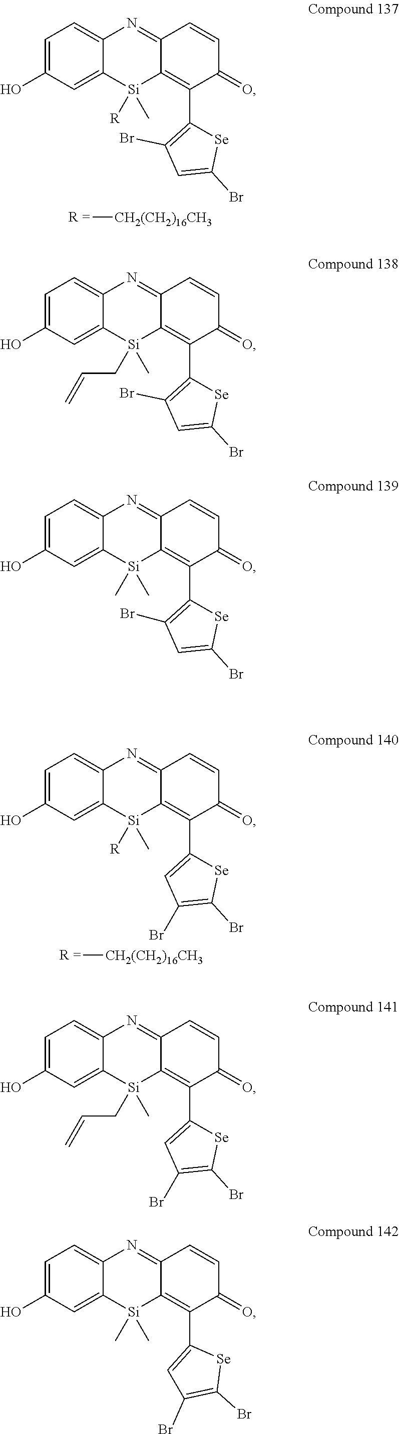

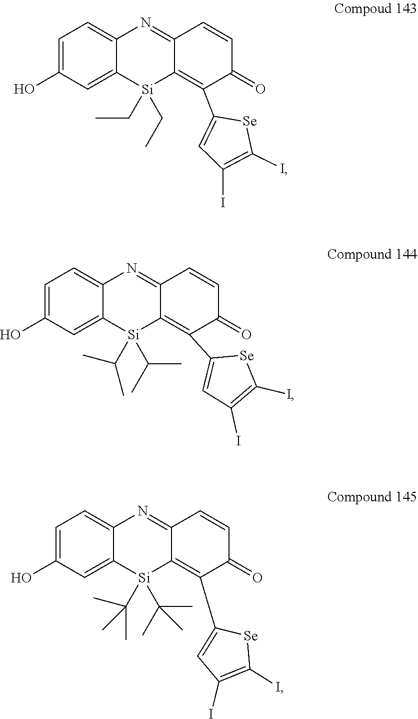 [SMILES: CC[Si]1(CC)C2=C(c3cc(I)c(I)[se]3)C(=O)C=CC2=Nc2ccc(O)cc21 ; CC(C)[Si]1(C(C)C)C2=C(c3cc(I)c(I)[se]3)C(=O)C=CC2=Nc2ccc(O)cc21 ; CC(C)(C)[Si]1(C(C)(C)C)C2=C(c3cc(I)c(I)[se]3)C(=O)C=CC2=Nc2ccc(O)cc21]

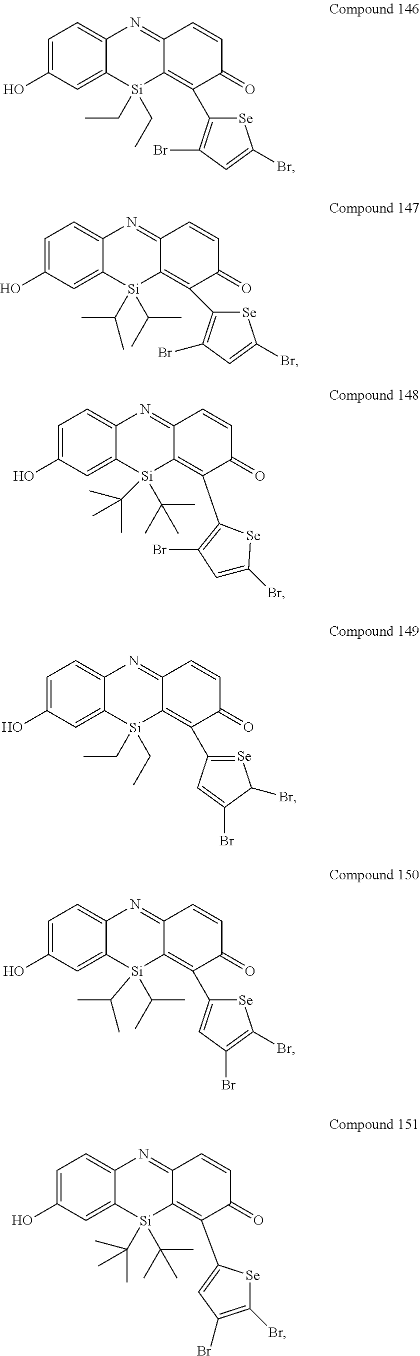

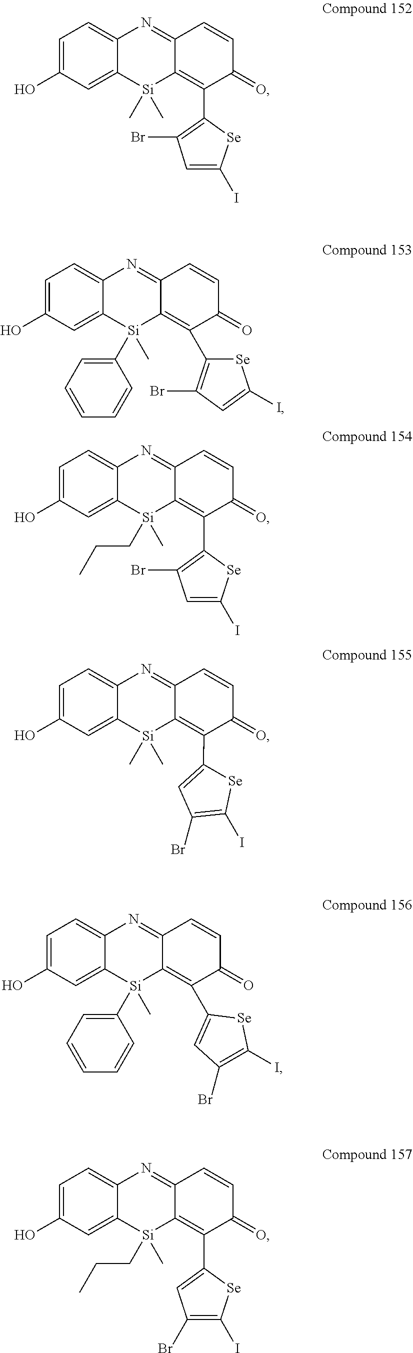

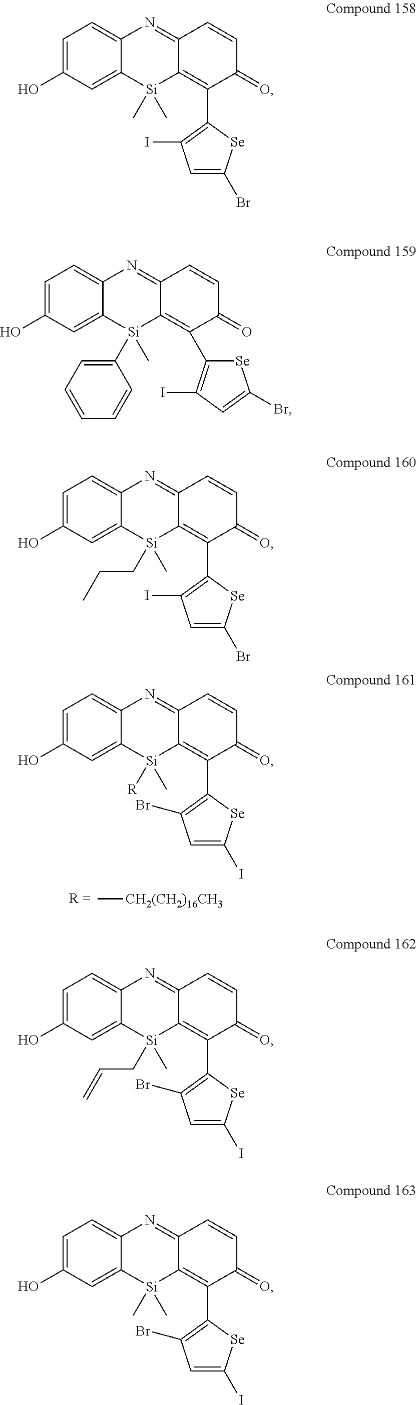

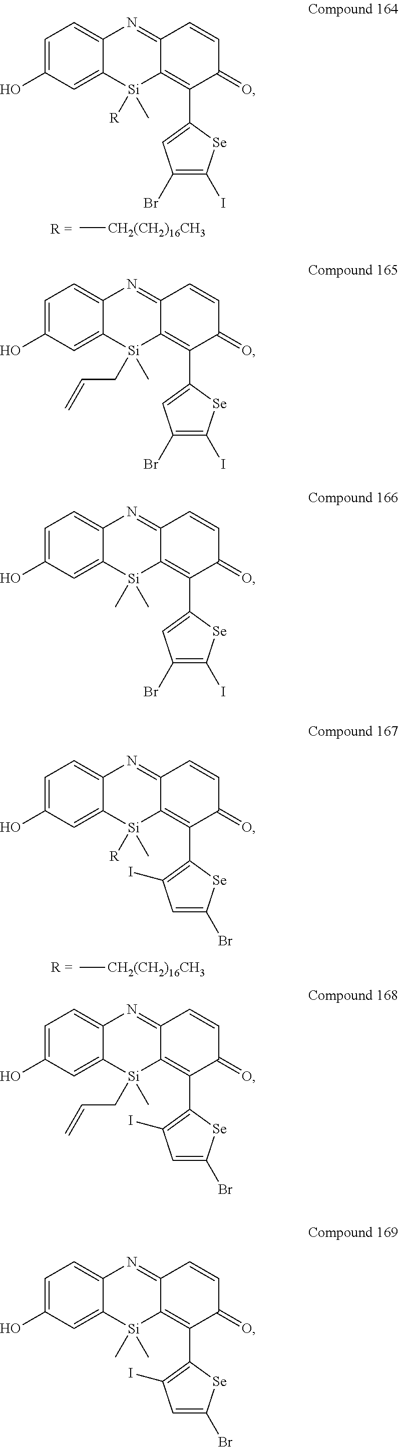

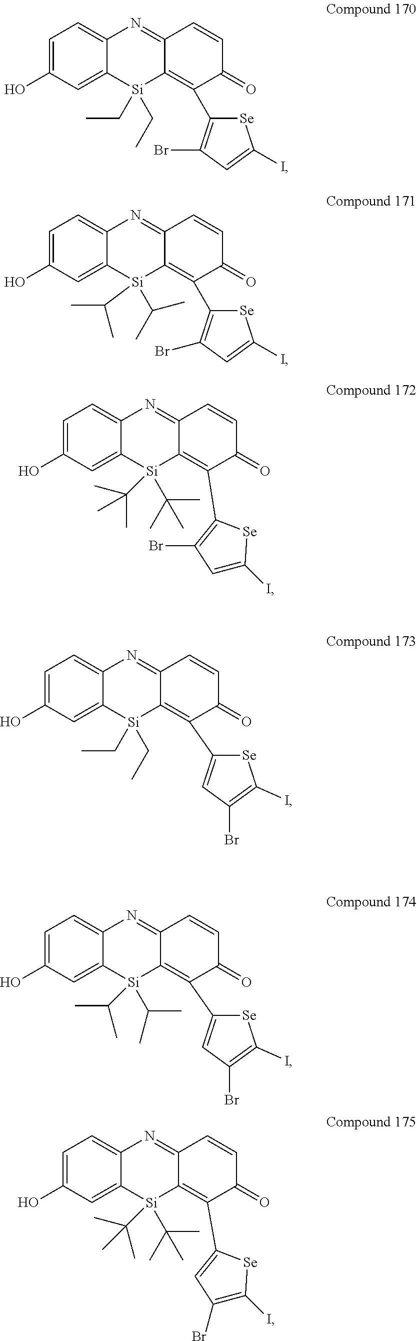

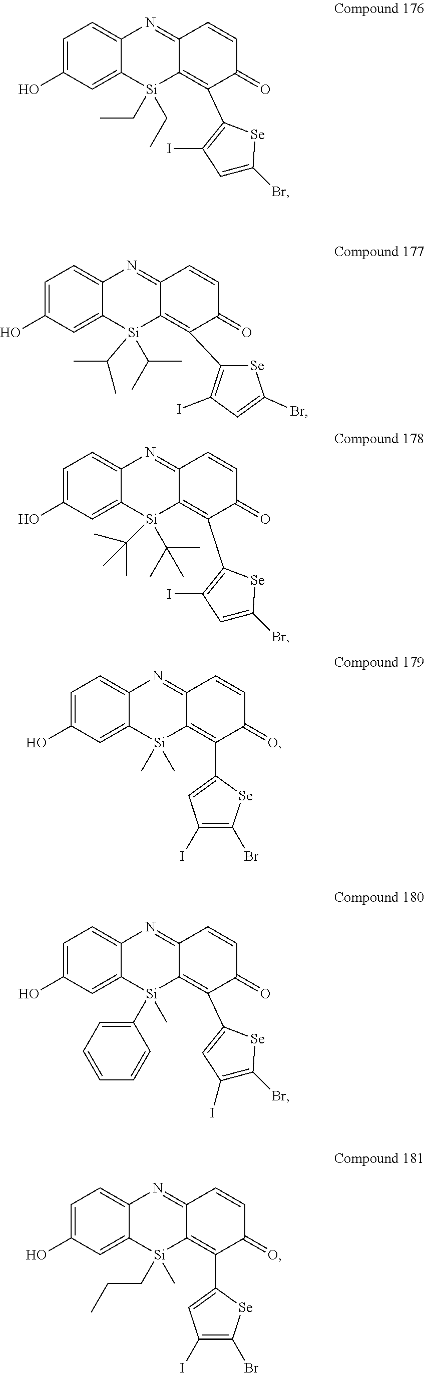

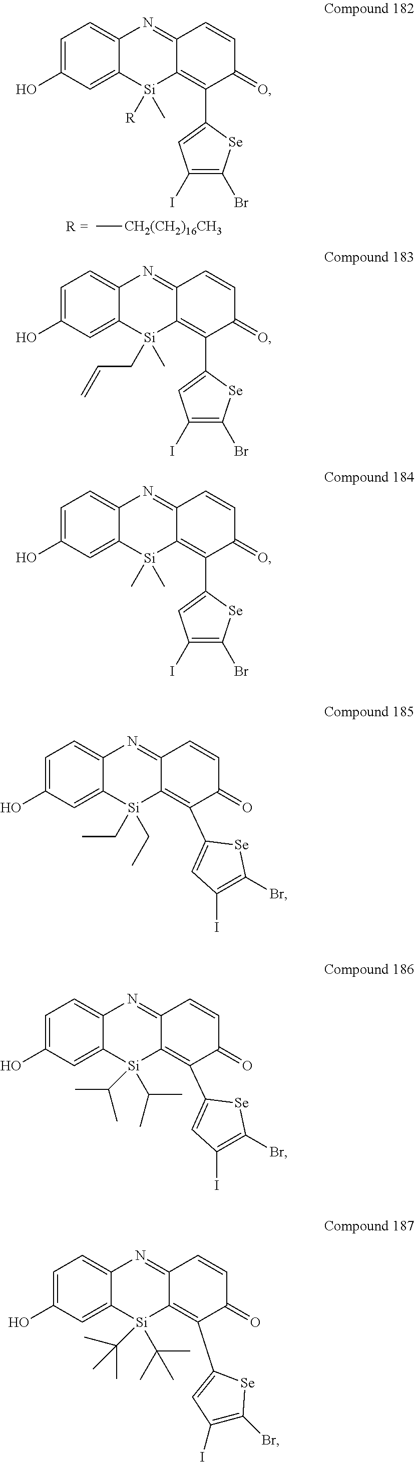

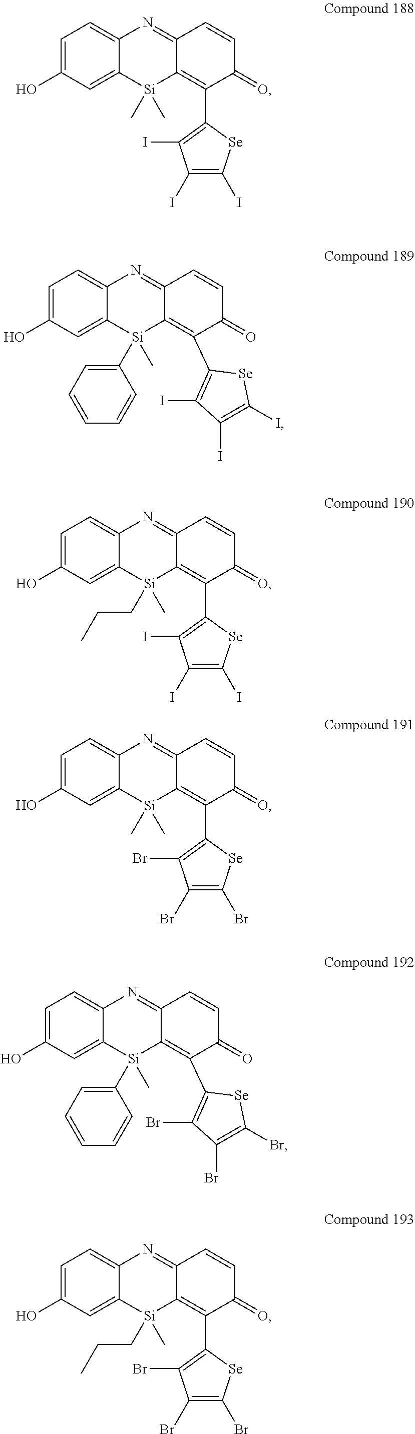 [SMILES: C[Si]1(C)C2=C(c3[se]c(I)c(I)c3I)C(=O)C=CC2=Nc2ccc(O)cc21 ; C[Si]1(c2ccccc2)C2=C(c3[se]c(I)c(I)c3I)C(=O)C=CC2=Nc2ccc(O)cc21 ; CCC[Si]1(C)C2=C(c3[se]c(I)c(I)c3I)C(=O)C=CC2=Nc2ccc(O)cc21 ; C[Si]1(C)C2=C(c3[se]c(Br)c(Br)c3Br)C(=O)C=CC2=Nc2ccc(O)cc21 ; C[Si]1(c2ccccc2)C2=C(c3[se]c(Br)c(Br)c3Br)C(=O)C=CC2=Nc2ccc(O)cc21 ; CCC[Si]1(C)C2=C(c3[se]c(Br)c(Br)c3Br)C(=O)C=CC2=Nc2ccc(O)cc21]

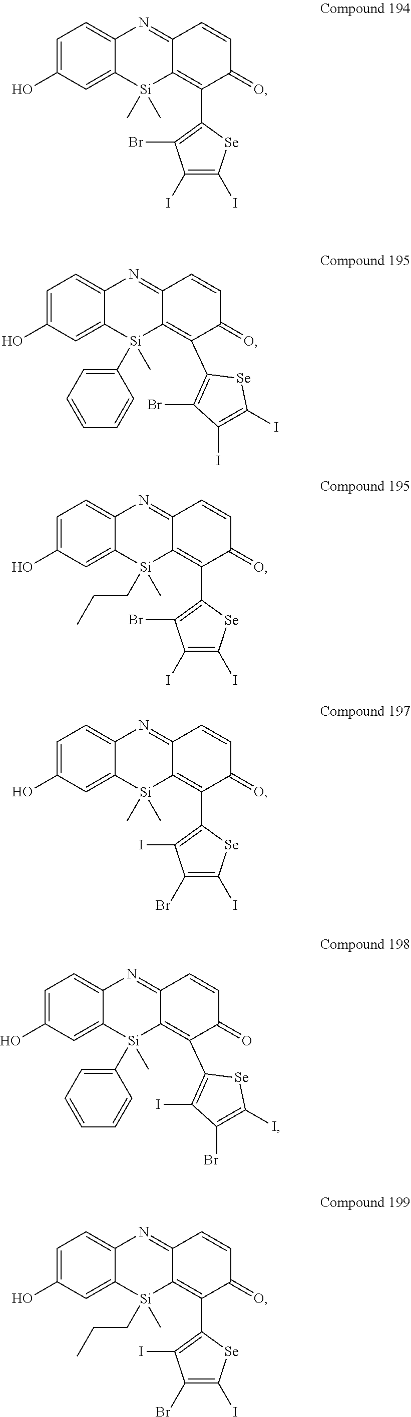

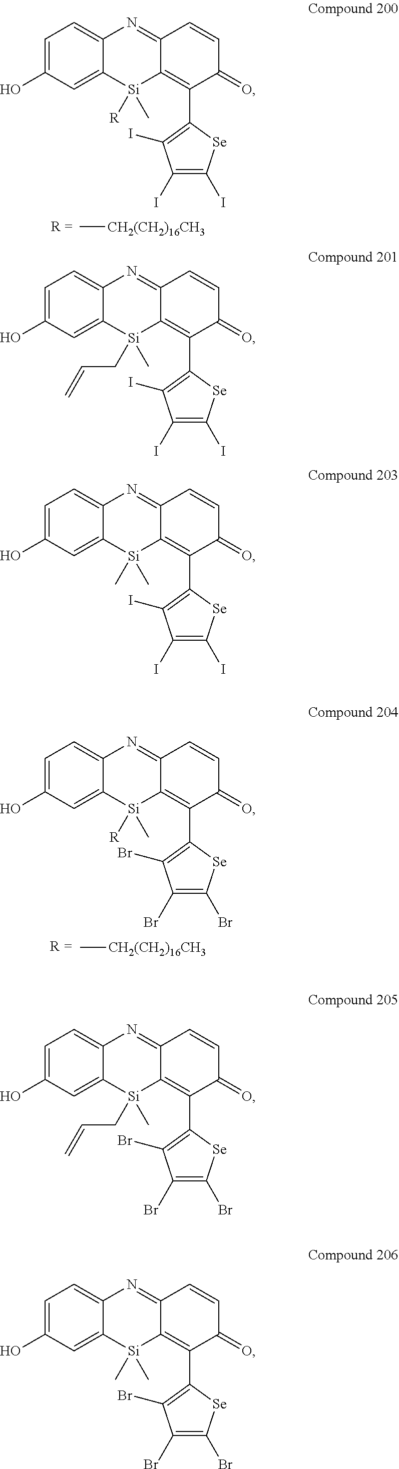

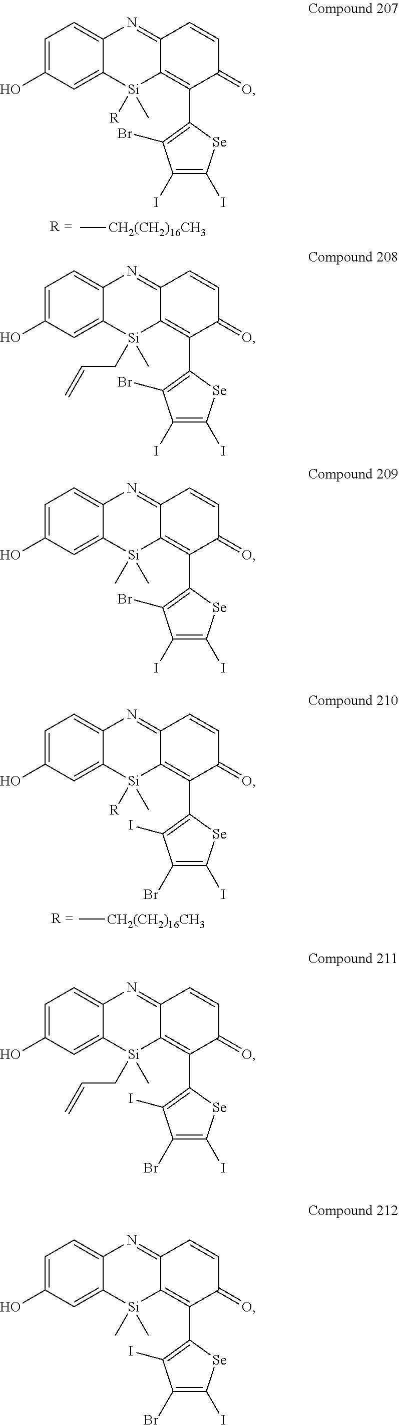

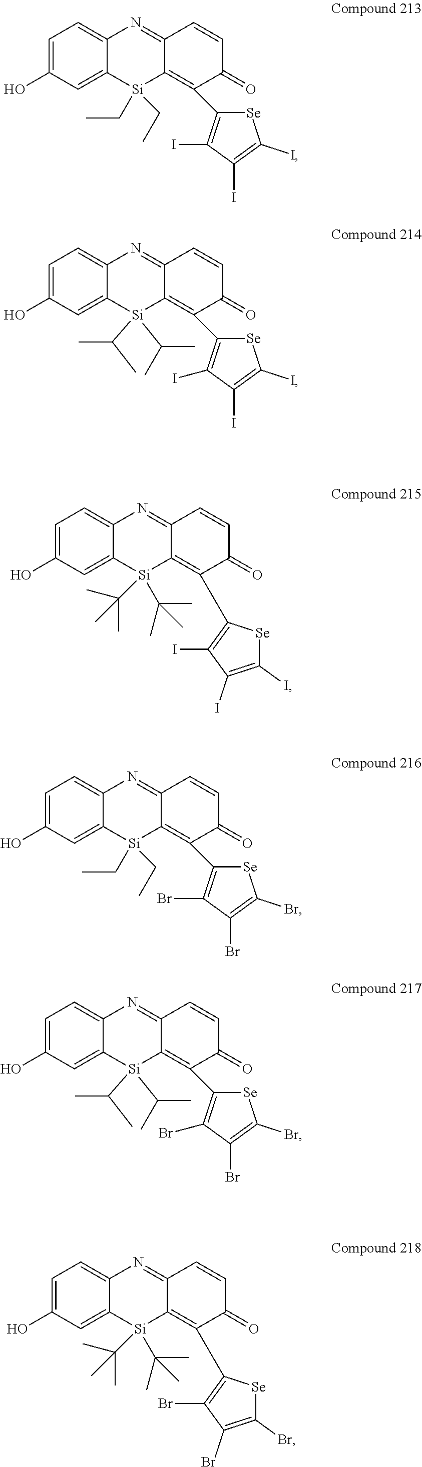 [SMILES: CC[Si]1(CC)C2=C(c3[se]c(I)c(I)c3I)C(=O)C=CC2=Nc2ccc(O)cc21 ; CC(C)[Si]1(C(C)C)C2=C(c3[se]c(I)c(I)c3I)C(=O)C=CC2=Nc2ccc(O)cc21 ; CC(C)(C)[Si]1(C(C)(C)C)C2=C(c3[se]c(I)c(I)c3I)C(=O)C=CC2=Nc2ccc(O)cc21 ; CC[Si]1(CC)C2=C(c3[se]c(Br)c(Br)c3Br)C(=O)C=CC2=Nc2ccc(O)cc21 ; CC(C)[Si]1(C(C)C)C2=C(c3[se]c(Br)c(Br)c3Br)C(=O)C=CC2=Nc2ccc(O)cc21 ; CC(C)(C)[Si]1(C(C)(C)C)C2=C(c3[se]c(Br)c(Br)c3Br)C(=O)C=CC2=Nc2ccc(O)cc21]

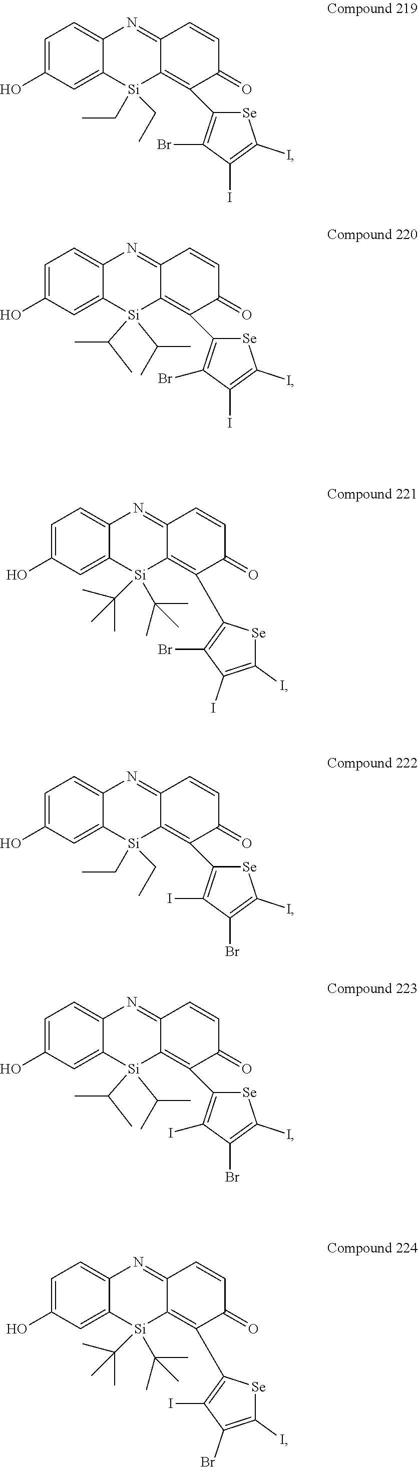 [SMILES: CC[Si]1(CC)C2=C(c3[se]c(I)c(I)c3Br)C(=O)C=CC2=Nc2ccc(O)cc21 ; CC(C)[Si]1(C(C)C)C2=C(c3[se]c(I)c(I)c3Br)C(=O)C=CC2=Nc2ccc(O)cc21 ; CC(C)(C)[Si]1(C(C)(C)C)C2=C(c3[se]c(I)c(I)c3Br)C(=O)C=CC2=Nc2ccc(O)cc21 ; CC[Si]1(CC)C2=C(c3[se]c(I)c(Br)c3I)C(=O)C=CC2=Nc2ccc(O)cc21 ; CC(C)[Si]1(C(C)C)C2=C(c3[se]c(I)c(Br)c3I)C(=O)C=CC2=Nc2ccc(O)cc21 ; CC(C)(C)[Si]1(C(C)(C)C)C2=C(c3[se]c(I)c(Br)c3I)C(=O)C=CC2=Nc2ccc(O)cc21]

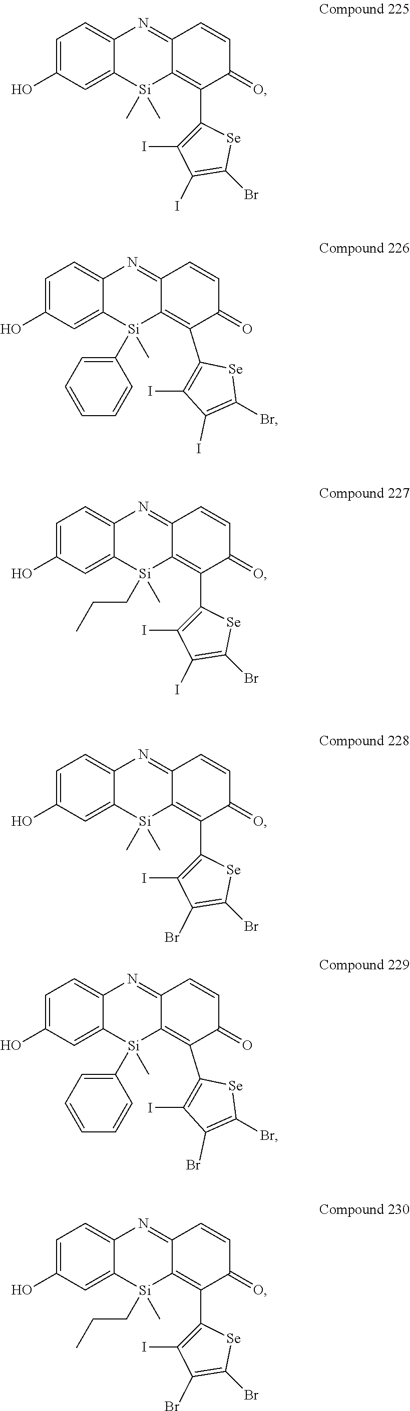

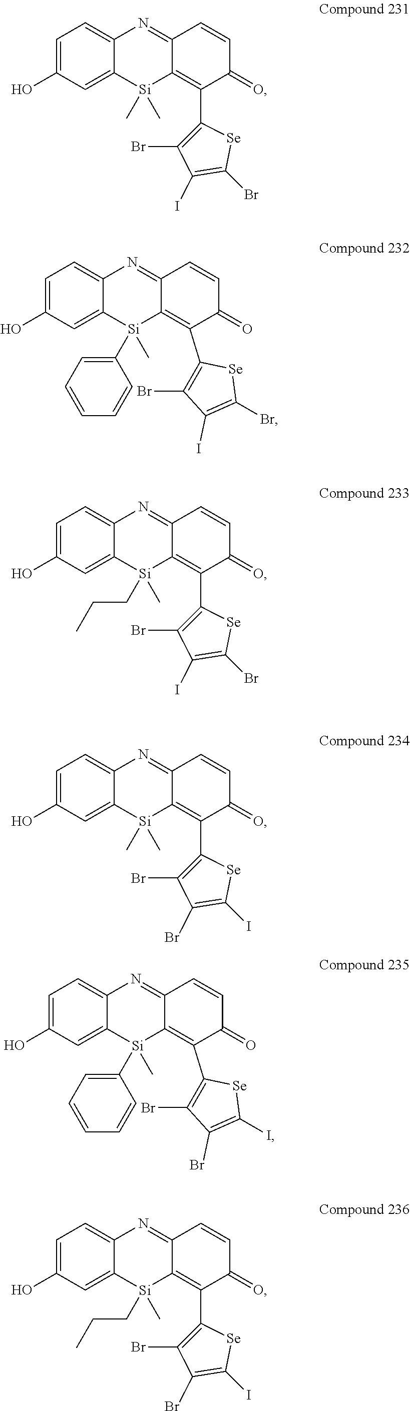

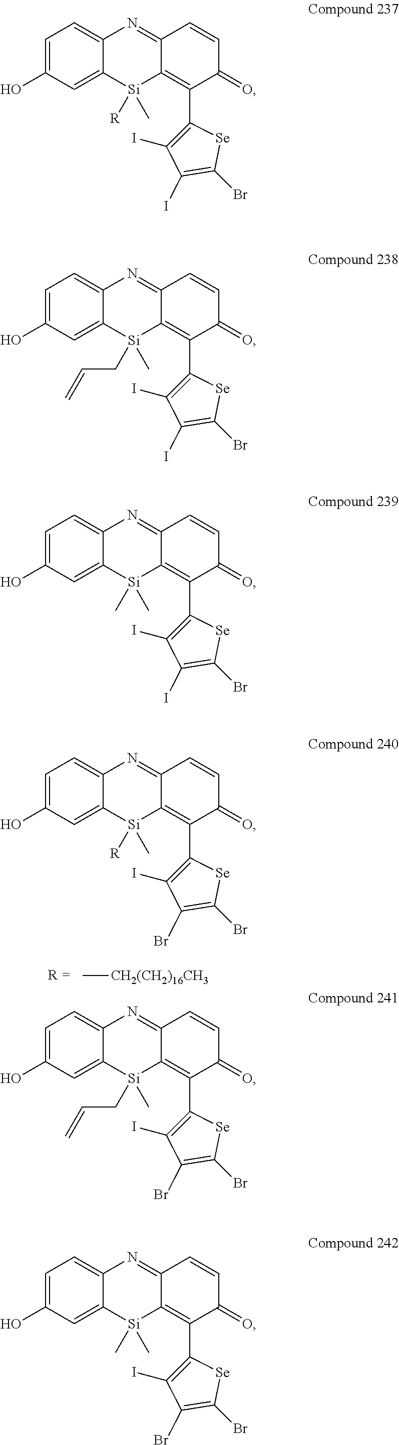

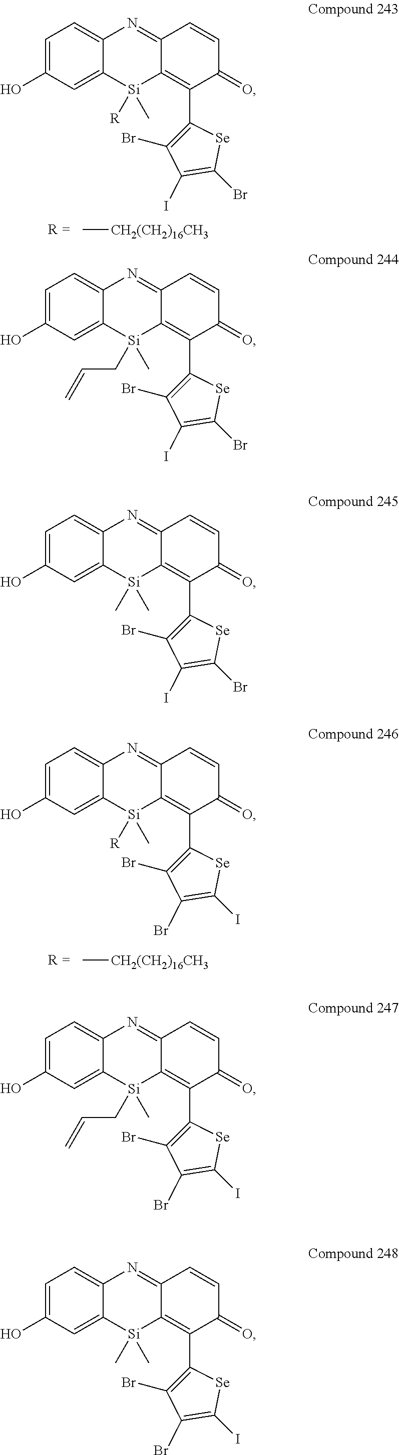

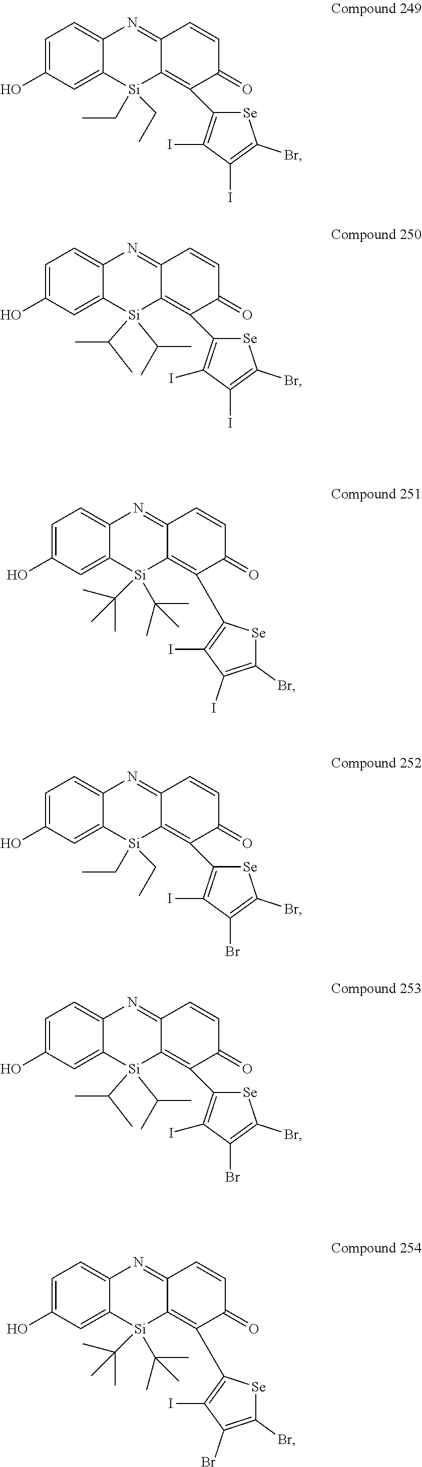 [SMILES: CC[Si]1(CC)C2=C(c3[se]c(Br)c(I)c3I)C(=O)C=CC2=Nc2ccc(O)cc21 ; CC(C)[Si]1(C(C)C)C2=C(c3[se]c(Br)c(I)c3I)C(=O)C=CC2=Nc2ccc(O)cc21 ; CC(C)(C)[Si]1(C(C)(C)C)C2=C(c3[se]c(Br)c(I)c3I)C(=O)C=CC2=Nc2ccc(O)cc21 ; CC[Si]1(CC)C2=C(c3[se]c(Br)c(Br)c3I)C(=O)C=CC2=Nc2ccc(O)cc21 ; CC(C)[Si]1(C(C)C)C2=C(c3[se]c(Br)c(Br)c3I)C(=O)C=CC2=Nc2ccc(O)cc21 ; CC(C)(C)[Si]1(C(C)(C)C)C2=C(c3[se]c(Br)c(Br)c3I)C(=O)C=CC2=Nc2ccc(O)cc21]

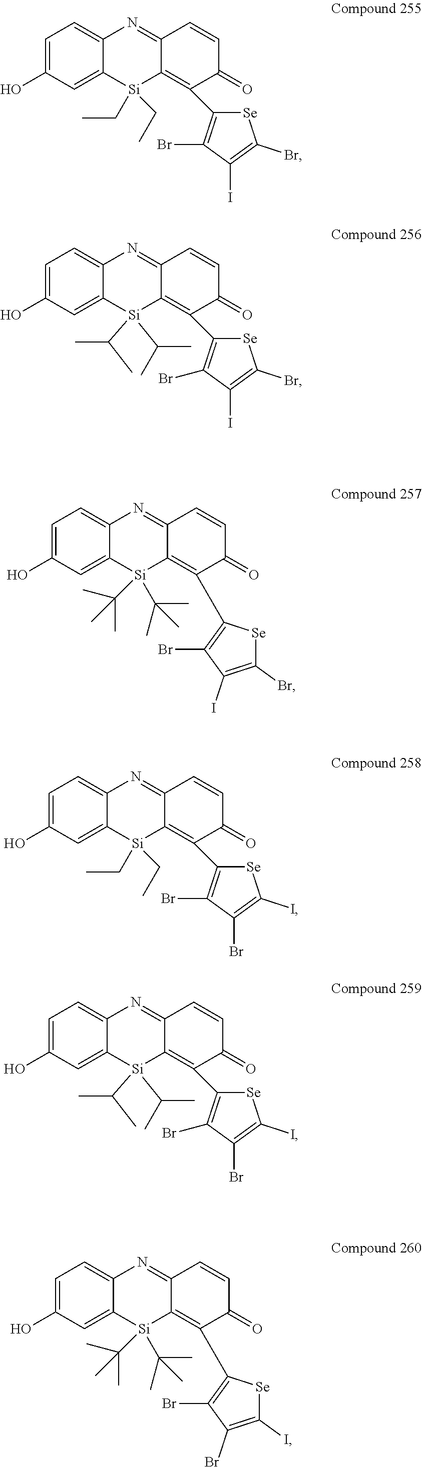 [SMILES: CC[Si]1(CC)C2=C(c3[se]c(Br)c(I)c3Br)C(=O)C=CC2=Nc2ccc(O)cc21 ; CC(C)[Si]1(C(C)C)C2=C(c3[se]c(Br)c(I)c3Br)C(=O)C=CC2=Nc2ccc(O)cc21 ; CC(C)(C)[Si]1(C(C)(C)C)C2=C(c3[se]c(Br)c(I)c3Br)C(=O)C=CC2=Nc2ccc(O)cc21 ; CC[Si]1(CC)C2=C(c3[se]c(I)c(Br)c3Br)C(=O)C=CC2=Nc2ccc(O)cc21 ; CC(C)[Si]1(C(C)C)C2=C(c3[se]c(I)c(Br)c3Br)C(=O)C=CC2=Nc2ccc(O)cc21 ; CC(C)(C)[Si]1(C(C)(C)C)C2=C(c3[se]c(I)c(Br)c3Br)C(=O)C=CC2=Nc2ccc(O)cc21]

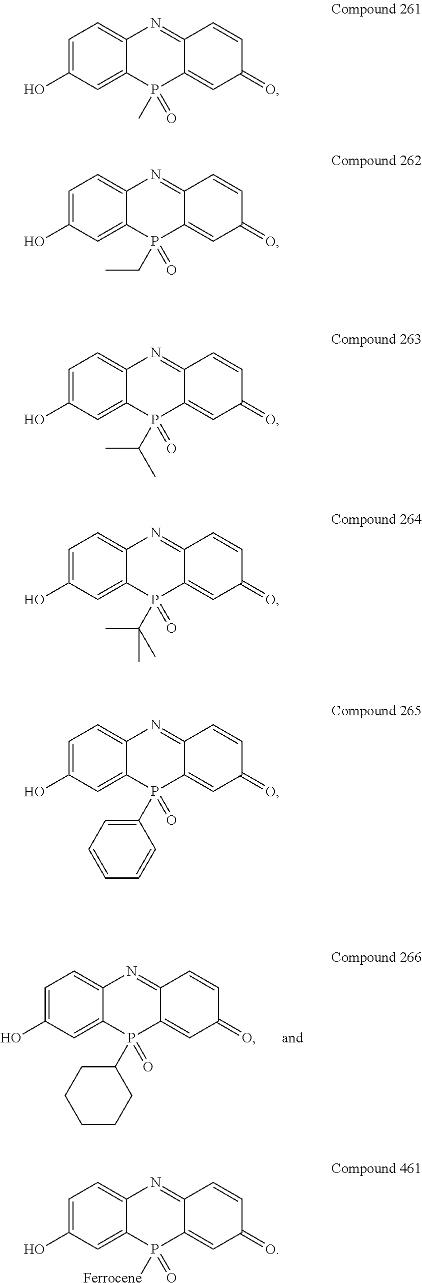

3. The NIR absorbing photosensitizer according to claim 1, comprising formula I or J or K or L or M or N: 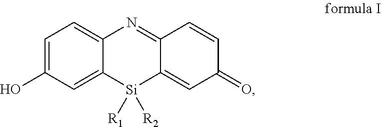
wherein; 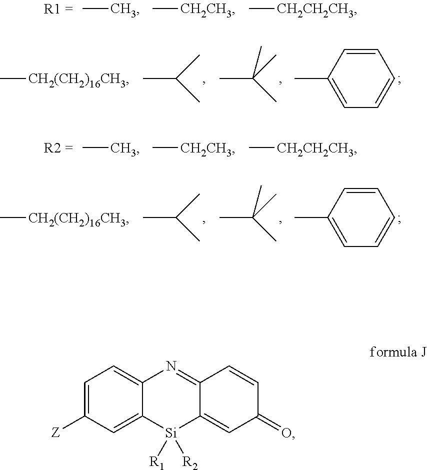
wherein; 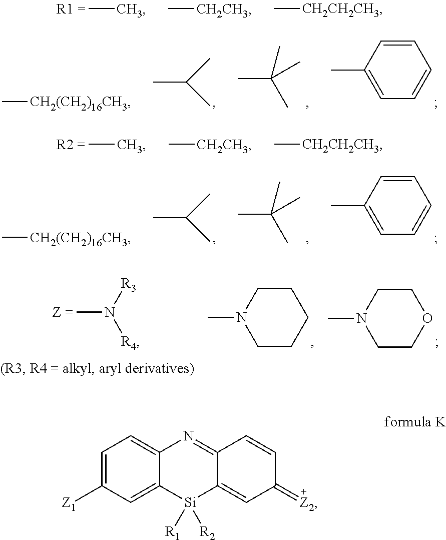
wherein; 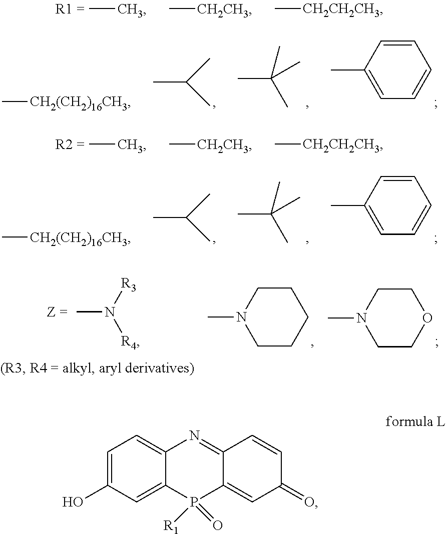
wherein; 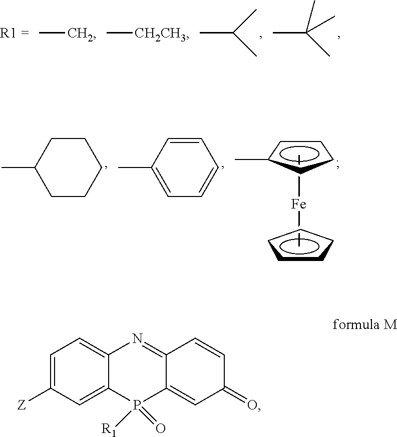
wherein; 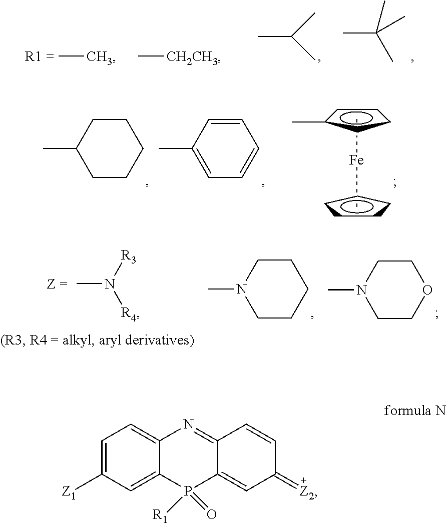
wherein;

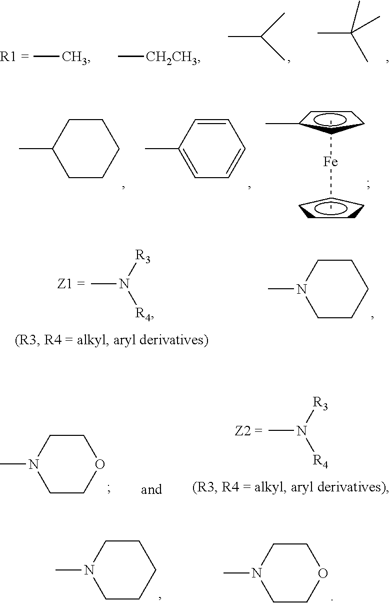

4. The NIR absorbing photosensitizer according to claim 3, comprising one selected from the group of:

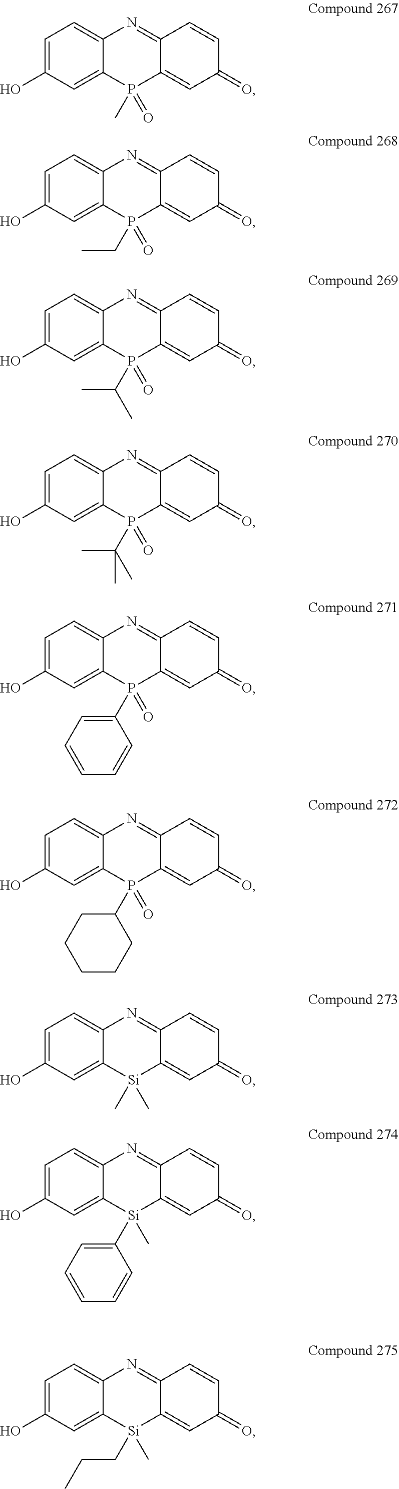

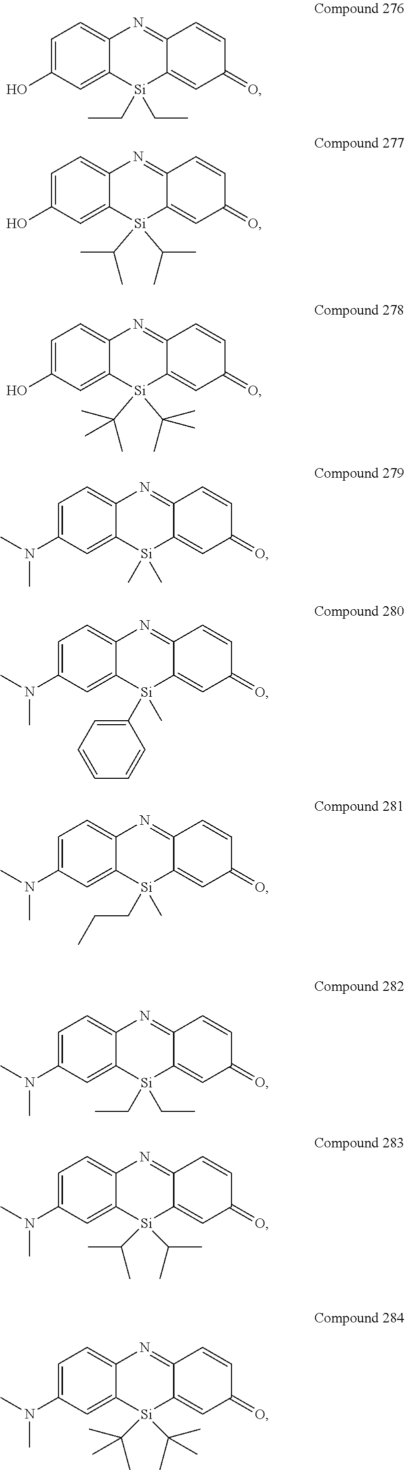

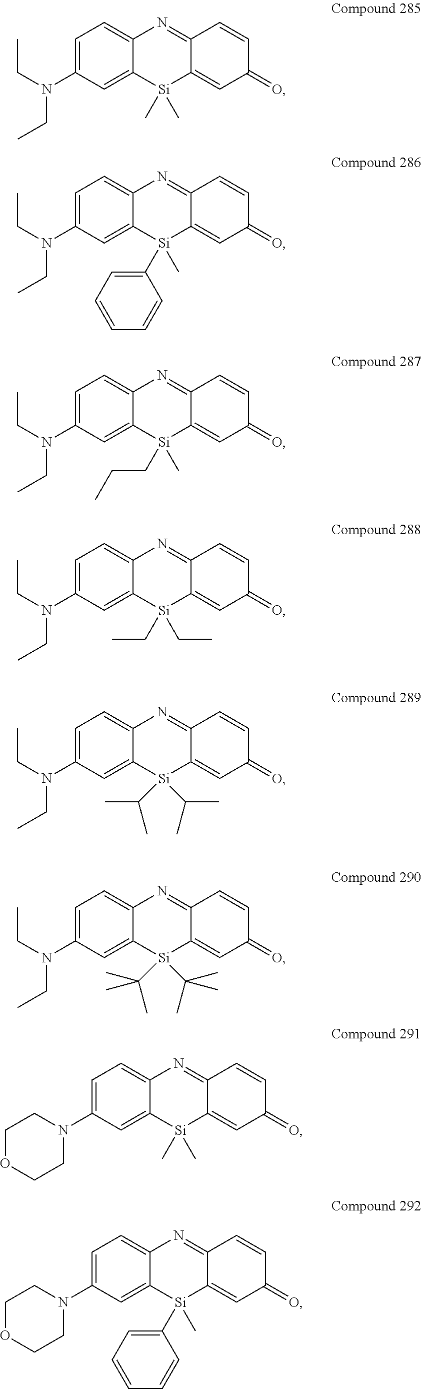

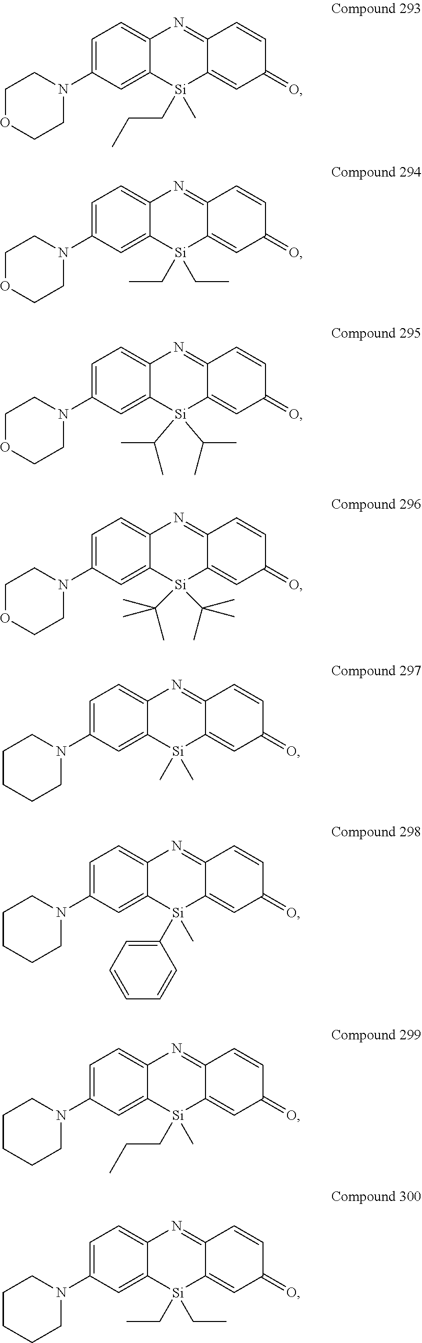

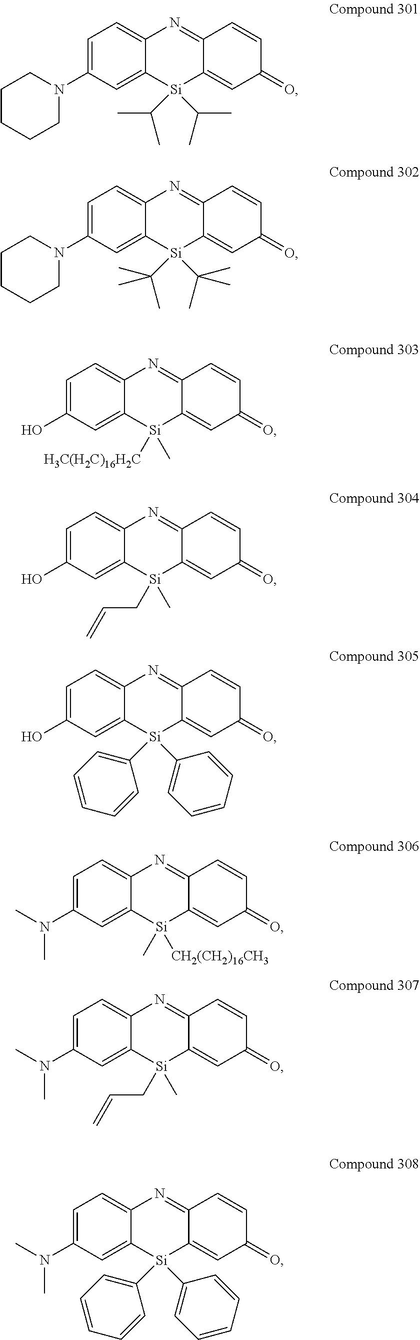

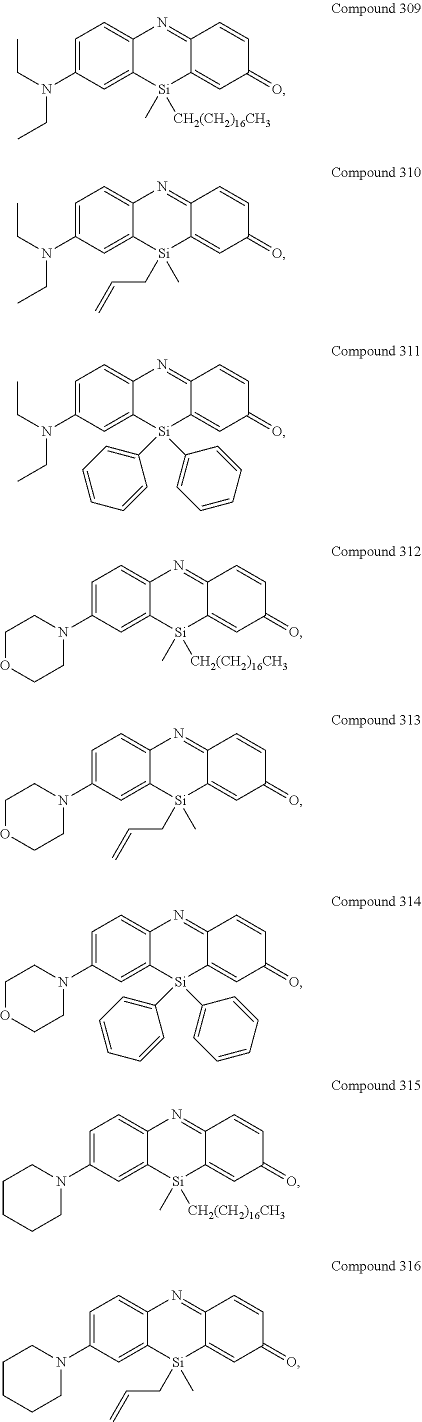

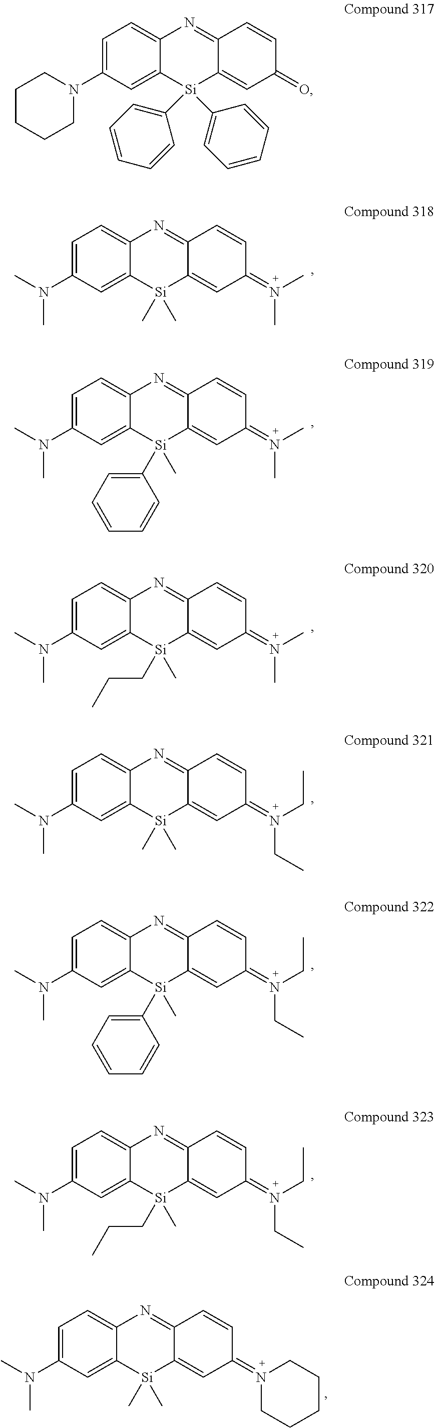

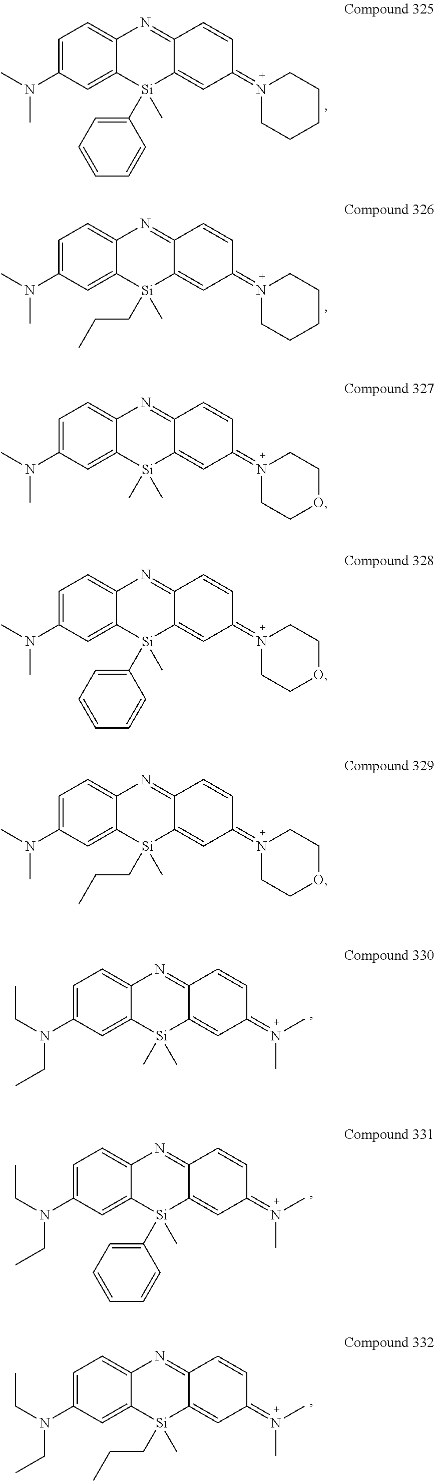

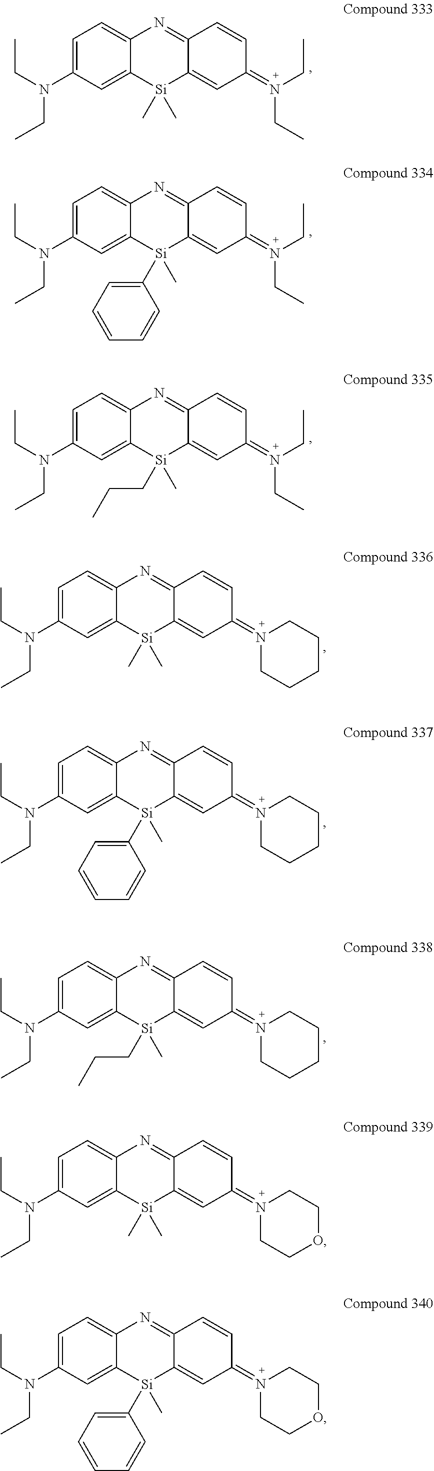

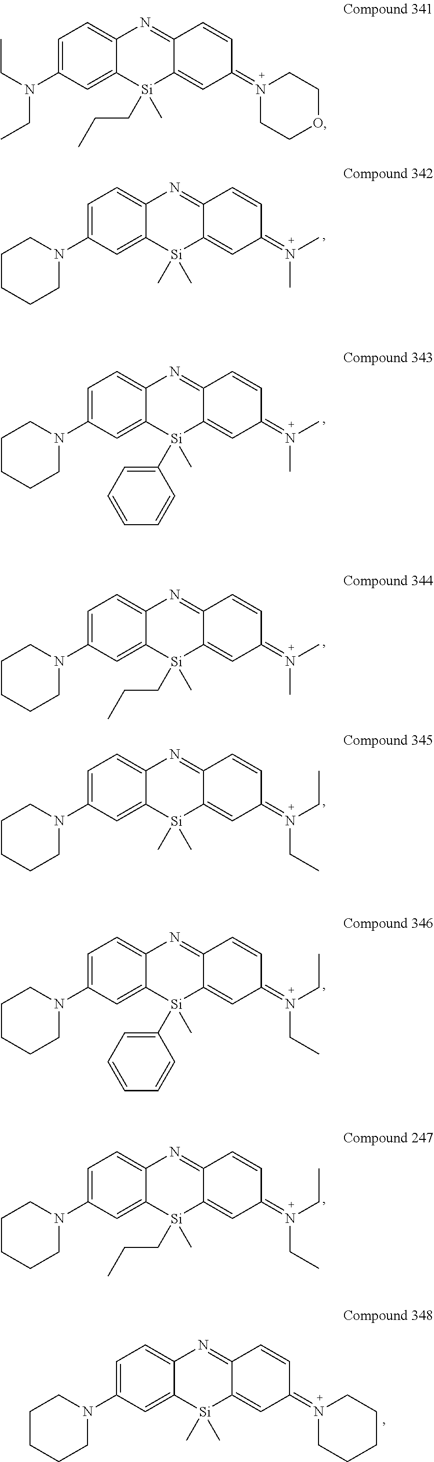

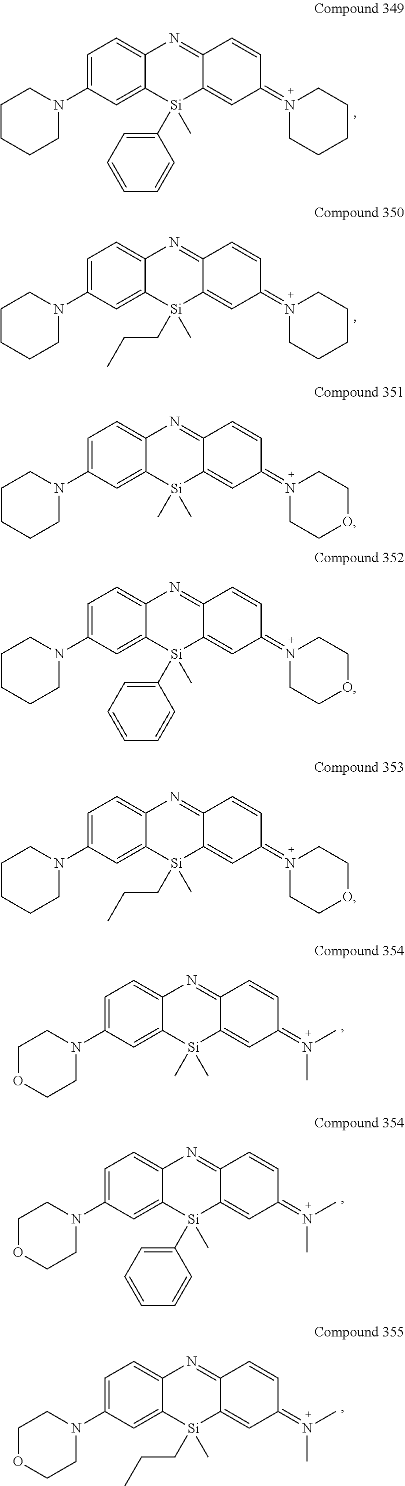

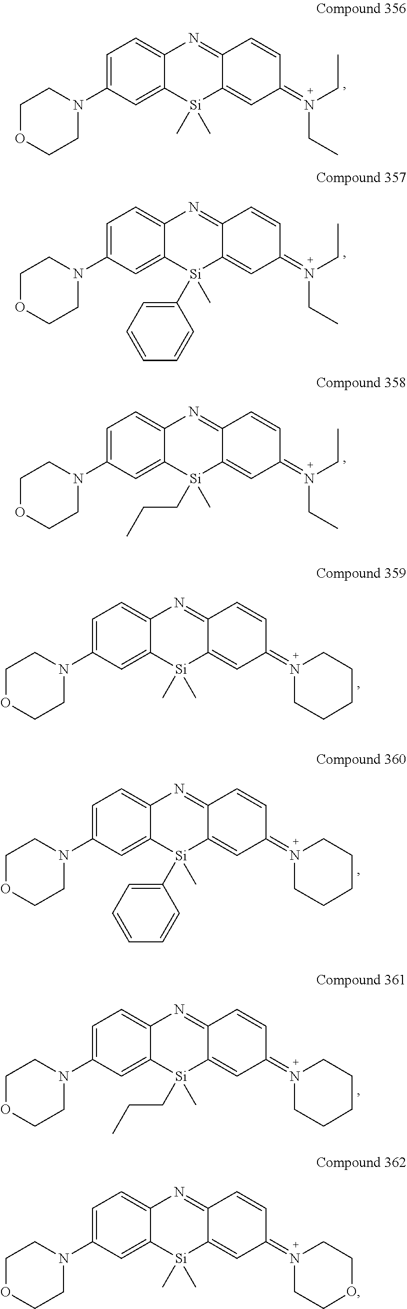

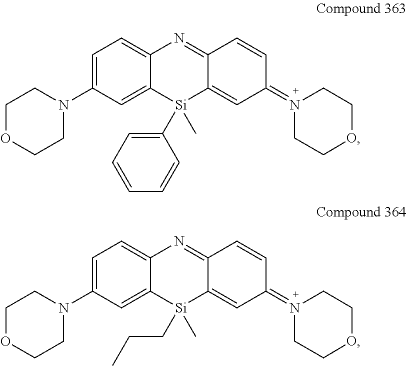

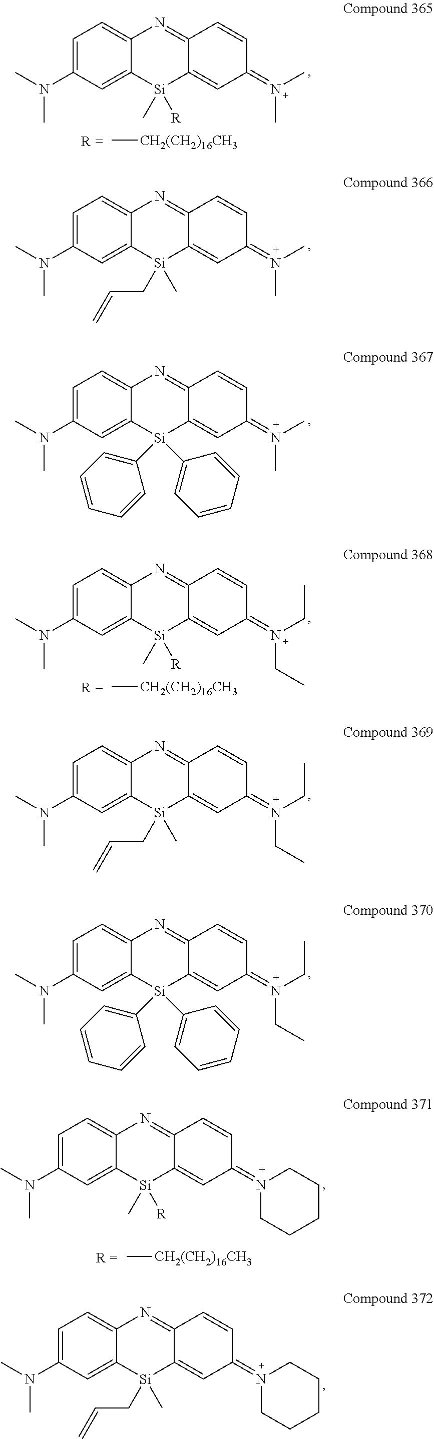

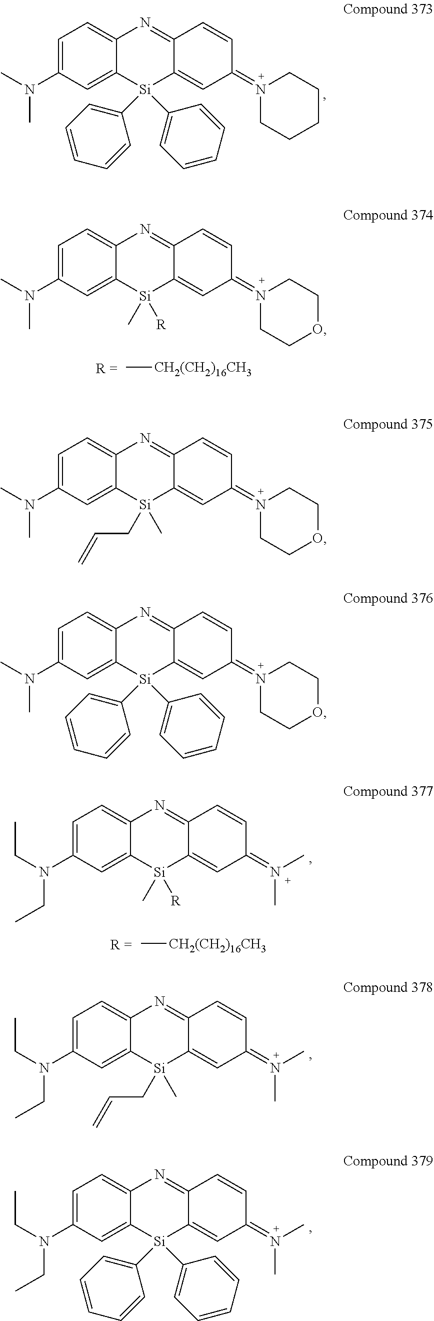

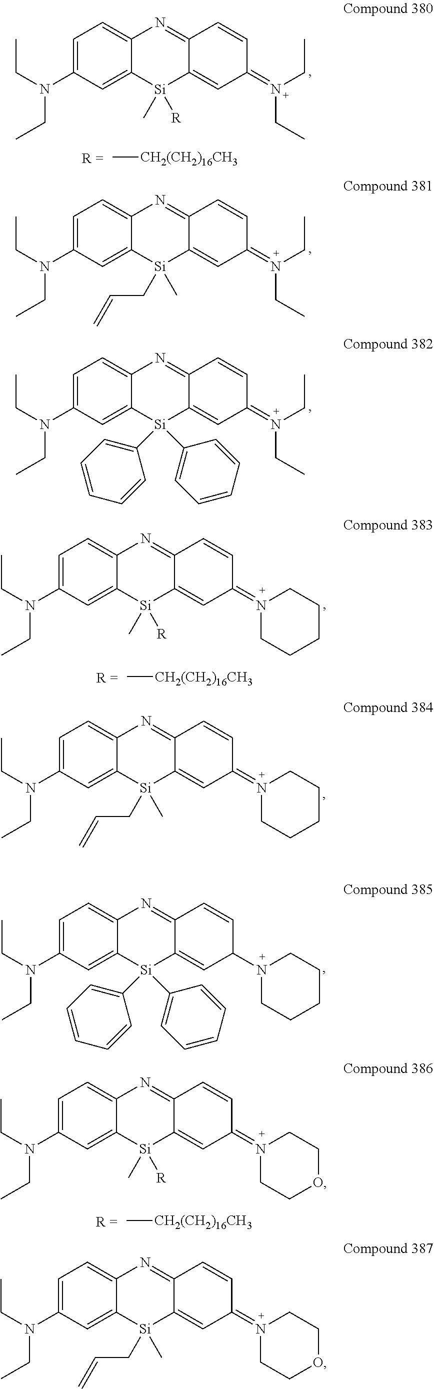

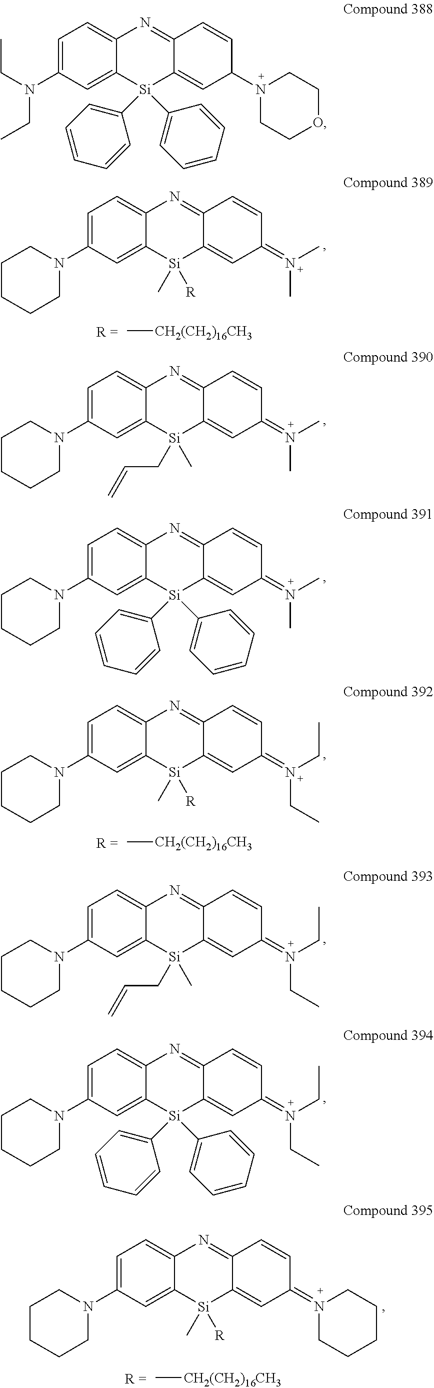

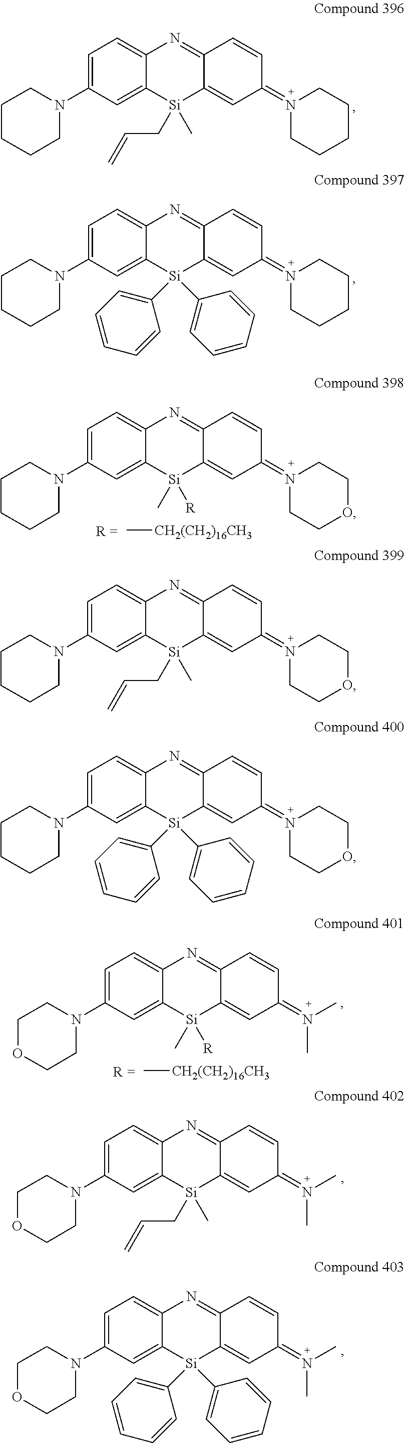

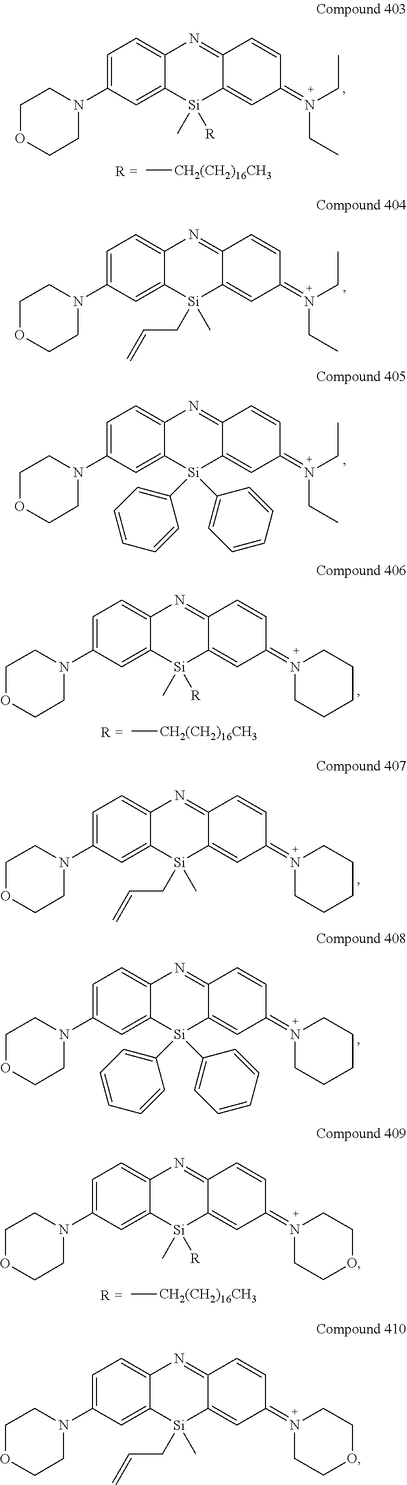

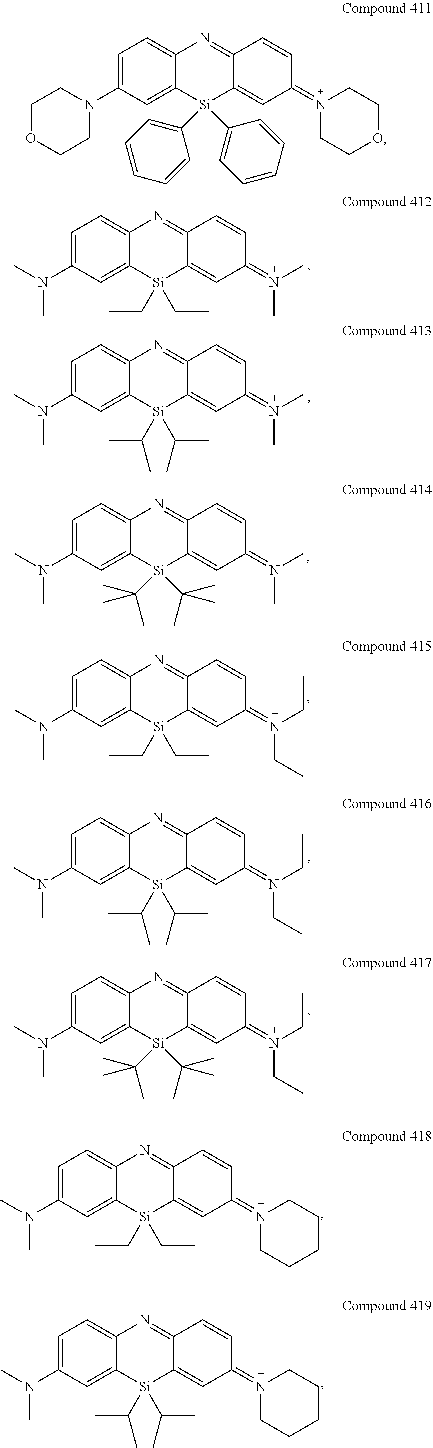

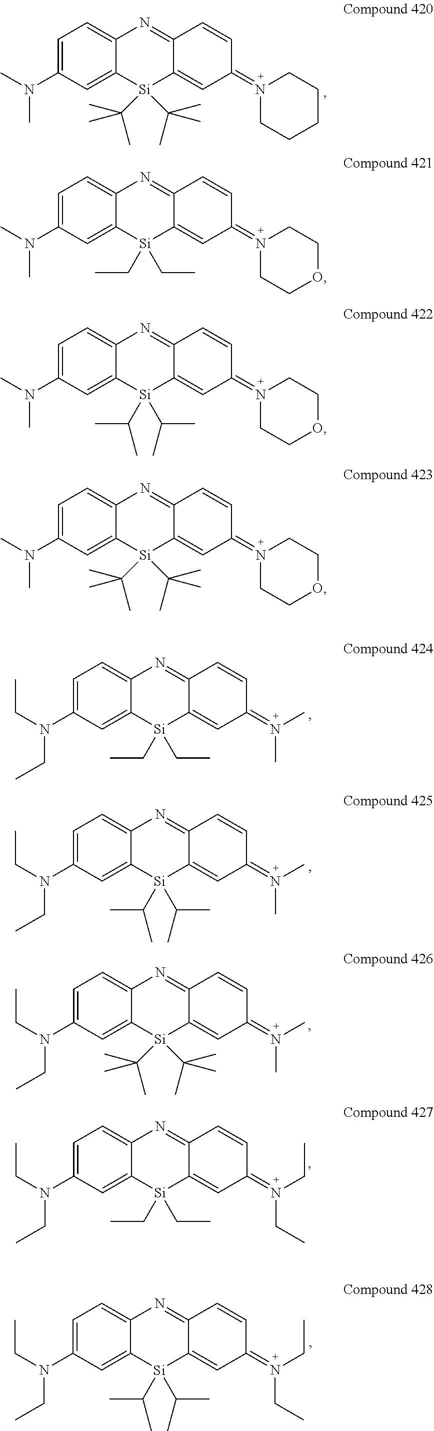

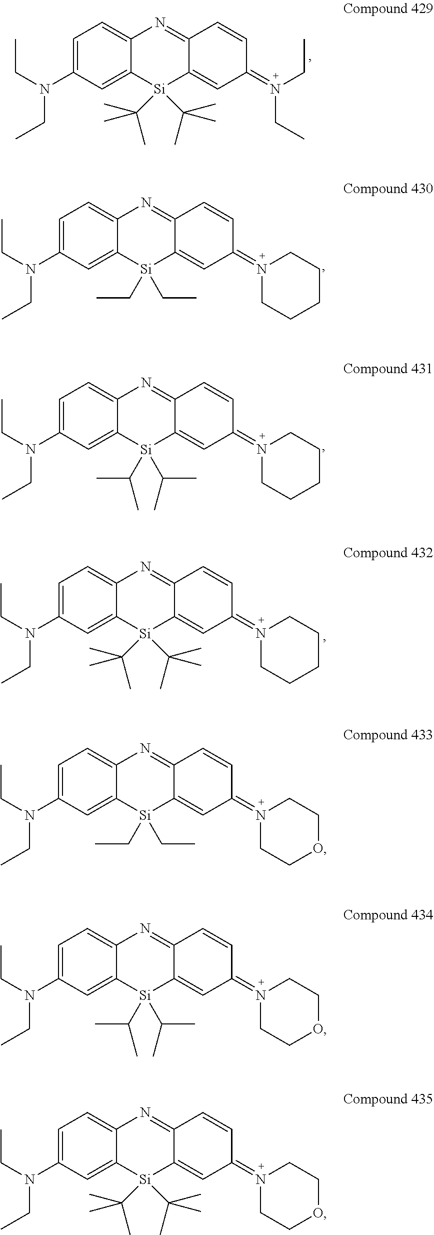

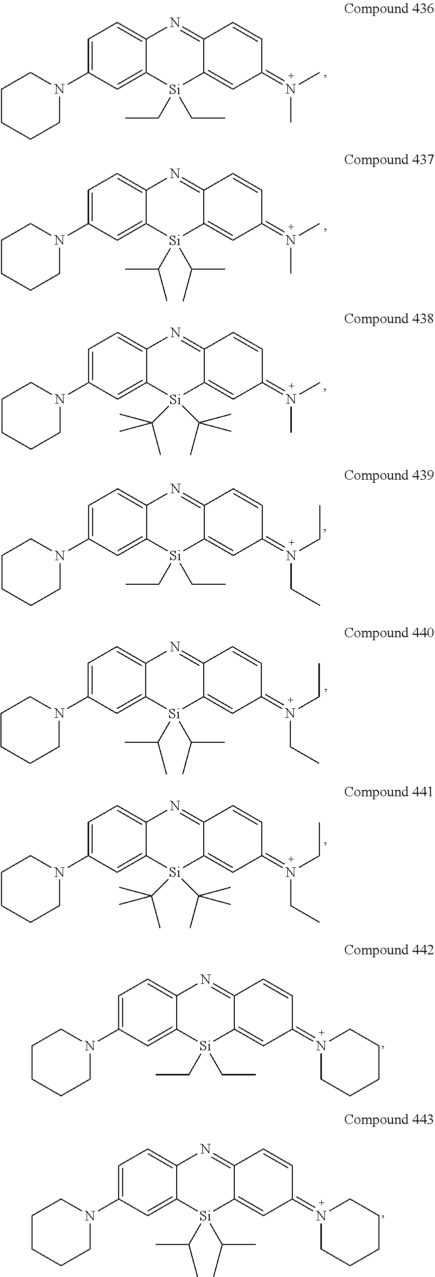

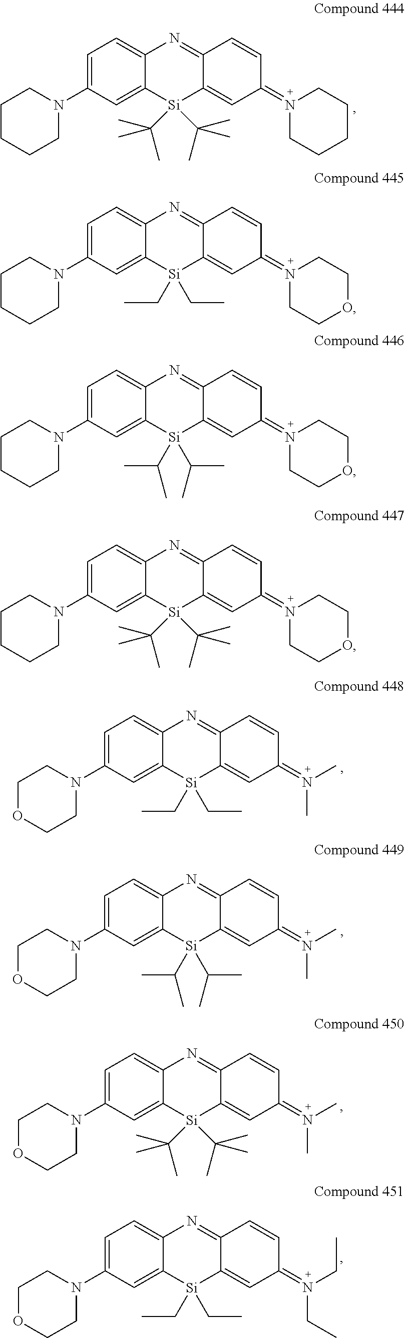

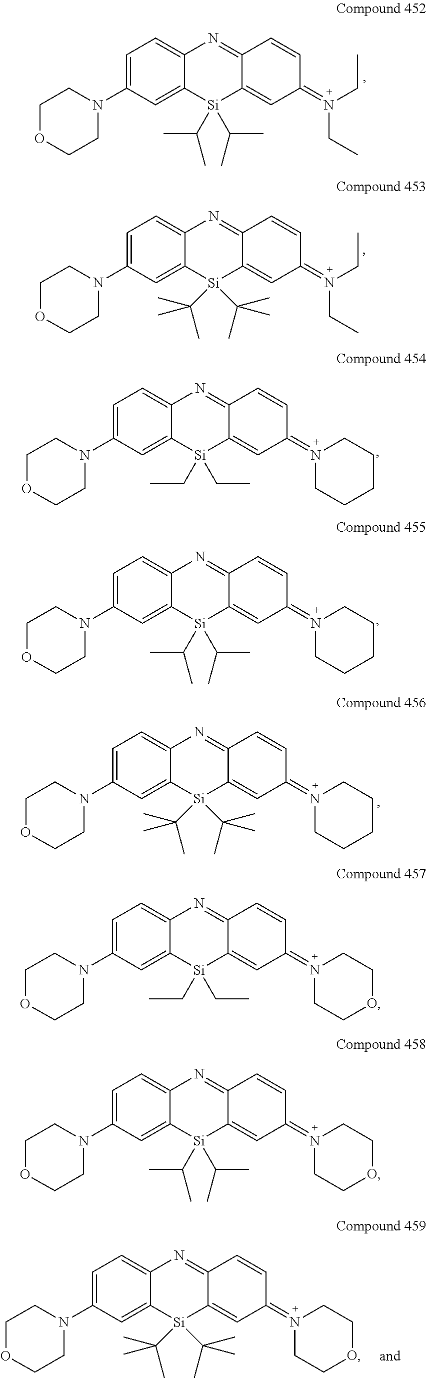

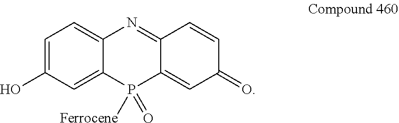

5. The NIR absorbing photosensitizer according to claim 1, wherein the NIR absorbing photosensitizer is mitochondria targeted.

6. The NIR absorbing photosensitizer according to claim 2, wherein the NIR absorbing photosensitizer is mitochondria targeted.

7. The NIR absorbing photosensitizer according to claim 1, wherein the NIR absorbing photosensitizer is water-soluble.

8. The NIR absorbing photosensitizer according to claim 2, wherein the NIR absorbing photosensitizer is water-soluble.

9. A method of using a compound according to claim 3 for an imaging of cancer cells.

10. A method of using a compound according to claim 4 for an imaging of cancer cells.
